SEQUENCE LISTING (20 sequences)

SEQ ID NO: 1 - DNA, 5921 bp, Homo sapiens
agcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccccgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggaccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgccaaaggcgatgctggtccccctgggcctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccccggtcccatgggcccccctggattggctggaccccctggtgaatctggacgtgagggggctcctgctgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcccgtcggccccgtcggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggaagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctcaagggctccaacgagatcgagatccgcgccgagggcaacagccgcttcacctacagcgtcactgtcgatggctgcacgagtcacaccggagcctggggcaagacagtgattgaatacaaaaccaccaagtcctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaacccccccaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaaaaagaataaataaataagtttttaaaaaaggaagcttggtccacttgcttgaagacccatgcgggggtaagtccctttctgcccgttgggttatgaaaccccaatgctgccctttctgctcctttctccacaccccccttggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcatcccttgtgccgcaggctgggcgggagagactgttctgttctgttccttgtgtaactgtgttgctgaaagactacctcgttcttgtcttgatgtgtcaccggggcaactgcctgggggcggggatgggggcagggtggaagcggctccccatttttataccaaaggtgctacatctatgtgatgggtggggtggggagggaatcactggtgctatagaaattgagatgcccccccaggccagcaaatgttcctttttgttcaaagtctatttttattccttgatattttttctttctttttttttttttttgtggatggggacttgtgaatttttctaaaggtgctatttaacatgggaggagagcgtgtgcgctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccctaggggtgggaggaagcaaaagactctgtacctattttgtatgtgtataataatttgagatgtttttaattattttgattgctggaataaagcatgtggaaatgacccaaacataa

SEQ ID NO: 2 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPAAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 3 - DNA, 50 bp, Artificial
  Artificial sequence = synthetic construct
gatcgctagcgccgccaccatgttcagctttgtggacctccggctcctgc

SEQ ID NO: 4 - DNA, 44 bp, Artificial
  Artificial sequence = synthetic construct
cgataagcttttacaggaagcagacagggccaacgtcgaagccg

SEQ ID NO: 5 - DNA, 2274 bp, Homo sapiens
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcaggtgcagatgagggagttaggcggagcctgtcccctgagctcagggaaggagatccctcctcctctcagcacctccccagcaccccctcatcacccagggtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 6 - protein, 757 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 7 - DNA, 49 bp, Artificial
  Artificial sequence = synthetic construct
gatcgctagcgccgccaccatggcgggtctgacggcggcggccccgcgg

SEQ ID NO: 8 - DNA, 44 bp, Artificial
  Artificial sequence = synthetic construct
gctaagatcttcattttctcttccggccacaagctttccccagg

SEQ ID NO: 9 - DNA, 1984 bp, Homo sapiens
ggatccagagatttagattttttataagctttcctgccaccgaaacgggtgtttgggacctcacgaggccctgttcattcttcgtcgctgcgctccccactctgtactggatgcatttactgacgttgttgtctccgtccccagagtatgaacccccaaggtgactcatgcagctgtgggtgcccggcatacagcatggtgactggaatggatgagcacccaataaacatttgttgcaggaatgcaggaggacgggcaggccagcaagcaggctgcctggtttttcccacatgggcttttctgggaaagaagagcttctatttttggaaagggctgctatgattgagaaaagttcatggcagcaaaaaaaggacagacgtcgggagggaaacactcctagttctcccagacaacacattttttaaaaagactccttcatctctttaataataacggtaacgacaatgacaatgatgattacttatgagtgcggctagtgccagccactgtgttgtcactgggcgagtaatgatctcattggatcttcacggtgggcgtgcggggctccagggacagcctgcgttcctgggctggctgggtgcagctctcttttcaggagagaaagctctcttggaggagctggaaaggtgcccgactccagccatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcacgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcctgagcgcggcccccacccggcggcggccagggacccccgaggcccccctctgcctttgagcttctcctctgctccaacagacaccttccactctgaggtctcaccttcgcctctgctgaagtctccccgcagccctctccacccagaggtctccctataccgagacccaccatccttccatcctgaggaccgccccaaccctcggagccccccactcagtaggtctgaaggcctccatttgtaccgaaacaccccgctcacgctgacagcctcctaggctccctgaggtacctttccacccagaccctccttccccaccccataagccctgagactcccgcctttgacctgacgatcttcccccttcccgccttcaggttcctcctaggcgctcagaggccgctctggggggttgcctcgagtccccccacccctccccacccaccaccgctcccgcggcaagccagcccgtgcaacggaagccaggccaactgccccgcgtcttcagctgtttcgcatccaccgccaccccactgagagctgctcctttgggggaatgtttggcaacctttgtgttacagattaaaaattcagcaattca

SEQ ID NO: 10 - protein, 240 aa, Homo sapiens
MLALLCSCLLLAAGASDAWTGEDSAEPNSDSAEWIRDMYAKVTEIWQEVMQRRDDDGTLHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 11 - DNA, 56 bp, Artificial
  Artificial sequence = synthetic construct
gatcgctagcgccgccaccatgctggcgctactgtgttcctgcctgctcctggcag

SEQ ID NO: 12 - DNA, 46 bp, Artificial
  Artificial sequence = synthetic construct
cgataagctttcaggcggccttgcactcgctctcgcgccgccgctt

SEQ ID NO: 13 - DNA, 931 bp, Homo sapiens
tttcgtcggcccgccccttggcttctgcactgatggtgggtggatgagtaatgcatccaggaagcctggaggcctgtggtttccgcacccgctgccacccccgcccctagcgtggacatttatcctctagcgctcaggccctgccgccatcgccgcagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaagctgaagcctgcacagtgtccaccctgttcccactcccatctttcttccggacaatgaaataaagagttaccacccagcagaaaaaaaaaaaaaaaa

SEQ ID NO: 14 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 15 - DNA, 55 bp, Artificial
  Artificial sequence = synthetic construct
gatcgctagcgccgccaccatggccccctttgagcccctggcttctggcatcctg

SEQ ID NO: 16 - DNA, 46 bp, Artificial
  Artificial sequence = synthetic construct
cgataagctttcaggctatctgggaccgcagggactgccaggtgca

SEQ ID NO: 17 - DNA, 4101 bp, Homo sapiens
atgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataa

SEQ ID NO: 18 - protein, 1366 aa, Homo sapiens [sequence omitted]

SEQ ID NO: 19 - DNA, 58 bp, Artificial
  Artificial sequence = synthetic construct
gatcgctagcgccgccaccatgctcagctttgtggatacgcggactttgttgctgctt

SEQ ID NO: 20 - DNA, 51 bp, Artificial
  Artificial sequence = synthetic construct
cgataagcttttatttgaaacagactgggccaatgtccacaaagaattcct

(19) United States
(12) Patent Application Publication
(10) Pub. No.: US 2008/0119433 A1
(43) Pub. Date: May 22, 2008

(54) Compositions and Methods for Genetic Modification of Cells Having Cosmetic Function to Enhance Cosmetic Appearance

(76) Inventor: Aaron Thomas Tabor, Winston-Salem, NC (US)

Correspondence Address: KILPATRICK STOCKTON LLP, 1001 WEST FOURTH STREET, WINSTON-SALEM, NC, 27101, US

(21) Appl. No.: 11/774,450

(22) Filed: Jul. 6, 2007

Related U.S. Application Data
(60) Provisional application No. 60/818,776, filed on Jul. 6, 2006.

(51) Int. Cl. A61K 31/7088; A61K 48/00; A61Q 19/00; C07H 21/04; C12N 15/63; C12N 15/87; C12N 15/88

(52) U.S. Cl. 514/44; 435/455; 435/458; 435/459; 536/23.5; 536/23.51

(57) ABSTRACT

Disclosed are methods and compositions to genetically modify substantially intact cells having cosmetic function to enhance the cosmetic appearance in mammals so as to enhance and/or maintain a biochemical and/or physiological process that has a positive effect on cosmetic appearance. The methods and compositions may provide cosmetic benefits such as reduced skin sagging, increased skin thickness, reduced wrinkles, increased skin thickness and collagen content, increased skin tone and elasticity, increased skin hydration, and improved skin texture and color.

### PRIORITY CLAIM TO RELATED INVENTIONS

This application claims priority to U.S. Provisional Patent Application Ser. No. 60/818,776, filed Jul. 6, 2006. The disclosure of U.S. Provisional Patent Application Ser. No. 60/818,776 is incorporated by reference herein in its entirety.

### FIELD OF INVENTION

The present invention relates to compositions and methods for cosmetic genetic modification of cells that have cosmetic function.

### BACKGROUND

Expression and/or levels of skin proteins and other biomolecules such as collagen, elastin, extracellular matrix proteins, proteoglycans, growth factors, endogenous antioxidant enzymes, and/or DNA repair enzymes, may decline substantially with age, to produce undesirable changes in cosmetic appearance. For example, fibroblast and kertinocyte responsiveness to growth factor stimulation may also decline with age. In contrast, certain proteins may increase to produce undesirable changes in skin and other cells having cosmetic function. For example, matrix metalloproteinase-1 (MMP-1) protein may increase in skin cells; as MMP-1 accelerates collagen breakdown, the build-up of MMP-1 can be detrimental to the skin.

Such changes may include sagging, thinning, or wrinkling of the skin. There are topical compositions that are formulated to improve the appearance of skin, but generally, such formulations require frequent and multiple applications (i.e., once or twice daily year round). Alternatively, invasive intervention (e.g., plastic surgery, laser resurfacing, and injection procedures) may help to reduce sagging and wrinkling of the skin. Still, many individuals are reluctant to undergo a surgical procedure for a non-therapeutic (i.e., cosmetic) reason. Thus, there is a need for cosmetic compositions and methods that offer a more permanent solution than many skin creams can offer, but that do not require plastic surgery or other types of invasive intervention.

Genetic therapy can provide a targeted approach for the improvement, treatment and maintenance of cells having cosmetic function. For example, certain genes appear to be involved in skin tissue maintenance and repair. Numerous growth factors, including epidermal growth factor (EGF), transforming growth factor (e.g., TGF-beta), fibroblast growth factor (FGF), insulin-like growth factor (IGF-1), keratinocyte growth factor (KGF), vascular endothelial growth factor (VEGF), and PDGF may be involved in wound healing (see e.g., Grazul-Bilska et al., Drugs of Today, 2003, 39:787-800; S. Werner and R. Grose; Physiol. Rev., 83:835-870, 2003). For example, a mixture of vascular endothelial growth factor (VEGF), platelet-derived growth factor (PDGF), insulin-like growth factor (IGF-1), granulocyte/macrophage colony-stimulating factor (GM-CSF), interleukin (IL-8, IL-6), tumor necrosis factor (TNF-alpha), transforming growth factor (TGB-beta and matrix proteins may be used to improve wound healing (reviewed in Jimenez and Jimenez, Am. J. Surgery, 2004, 187:56 S-64S). Also, insulin-like growth factor (IGF-1) may be used to increase muscle hypertrophy (Barton-Davis et al., Acta Physiol. Scand., 1999, 167:301-305). Thus, improving the production and function of specific proteins by molecular targeting of the genome of cells involved in cosmetic function may provide a means to improve the longevity and health of such cells.

### SUMMARY

Certain embodiments of the present invention address such cosmetic problems associated with aging and other unwanted changes that can occur in skin and other cells that have cosmetic function. Embodiments of the present invention provide methods and compositions for improving the cosmetic appearance of cells that have cosmetic function. As disclosed herein, embodiments of the methods and compositions of the present invention may comprise polynucleotide constructs that encode for nucleic acids and/or polypeptides that may act to improve and/or maintain the appearance of such cells. Alternatively or additionally, compositions comprising isolated polypeptides or biologically active derivatives thereto may be used. Thus, embodiments of the present invention address the problem of cosmetic degeneration of skin and other cells that have cosmetic function, and may provide compositions and methods for genetic modification of cells having cosmetic function. The present invention may be embodied in a variety of ways.

In one embodiment, the present invention may comprise a method for the cosmetic genetic modification of substantially intact cells having a cosmetic function in a subject comprising administering a polynucleotide encoding at least one of a nucleic acid or a polypeptide involved in maintaining the cells having cosmetic function to a least a portion of the cells such that the nucleic acid or polypeptide is expressed in the cells having cosmetic function to enhance and/or maintain a biochemical and/or physiological process that has a positive effect on cosmetic appearance.

In another embodiment, the present invention comprises a composition for genetically modifying substantially intact cells having cosmetic function in a subject. The composition may comprise an isolated polynucleotide encoding at least one of a nucleic acid, or a polypeptide involved in maintaining cells having cosmetic function. Or, the composition may comprise a polypeptide involved in maintaining cells having cosmetic function. The composition may further comprise a carrier for administration of the polynucleotide or polypeptide to a least a portion of the subject's cells having cosmetic function. In an embodiment the composition is formulated such that upon administration to a subject, the nucleic acid or polypeptide is expressed in the cells having cosmetic function so as to enhance and/or maintain a biochemical and/or physiological process that has a positive effect on cosmetic appearance.

Thus, embodiments of the present invention may comprise methods and compositions for the cosmetic genetic modification of cells having cosmetic function. In certain embodiments, the present invention may comprise methods and compositions for the in vivo transfection of recombinant polynucleotide constructs into cells having cosmetic function. In other embodiments, the invention may comprise methods and compositions for the ex vivo transfection of recombinant polynucleotide constructs into cells having cosmetic function followed by injection of the transfected cells into the skin or other cells that have cosmetic function. In yet other embodiments, polypeptides may be used in the methods and compositions of the present invention.

The recombinant constructs may encode for a variety of biomolecules that may be used to enhance the expression of proteins and other biomolecules that are beneficial to cells having a cosmetic function. In an embodiment, the composition may comprise a polypeptide. Or, the construct may comprise a recombinant DNA molecule that encodes for a polypeptide or other biomolecule that may modify the activity of genes in cells having cosmetic function. For example, in alternate embodiments, the polynucleotide may encode for a collagen polypeptide, an elastin polypeptide, a TIMP-1 polypeptide, or a superoxide dismutase (SOD) polypeptide.

Or, the recombinant constructs may encode for regulatory nucleic acid molecules, as for example, antisense oligodeoxynucleotides, or inhibitory RNAs (RNAi), ribozymes, triplex helix-forming oligonucleotides (TFOs) or peptide nucleic acid (PNA) that impact genes in cells having cosmetic function, as for example by downregulating expression of collagen and elastin degrading enzymes (e.g., matrix metalloproteinases, collagenases, gelatinases, elastases, stromelysins, serine proteases and/or membrane-type MMPs). For example, antisense oligodeoxynucleotides targeted against cysteine-rich 61 (CYR61/CCN1) which normally suppresses collagen 1 expression, and antisense oligodeoxynucleotides targeted against matrix metalloproteinases (MMPs) which normally accelerate collagen and elastin breakdown may be used.

In alternate embodiments, the polynucleotide construct may encode for a growth factor that improves cosmetic appearance. For example, the polynucleotide construct may encode for keratinocyte growth factor (KGF), insulin-like growth factor 1 (IGF-I), platelet derived growth factor (PDGF), hepatocyte growth factor (HGF), and/or transforming growth factor beta (TGF-beta).

The methods and compositions of the present invention may produce “genetic face and/or body lift” without the need for invasive surgical procedures. Such genetic modifications may restore or enhance a biochemical and/or physiological process that has a positive effect on cosmetic appearance (e.g. boosting collagen, elastin and/or superoxide dismutase production), or may reduce a skin cell process that has a negative cosmetic appearance (e.g. increased inhibition of collagenases by increased expression of inhibitors).

Other embodiments and further details regarding various aspects of the present invention are set forth in the following description and claims. It is to be understood that the invention is not limited in its application to the details set forth in the following description and claims, but is capable of other embodiments and of being practiced or carried out in various ways.

### BRIEF DESCRIPTION OF THE DRAWINGS

FIG. 1 shows sequences for procollagen DNA (COL1A1) (SEQ ID NO: 1) (panels A and B); protein (SEQ ID NO: 2) (panel C); and primers used to subclone the procollagen cDNA into Pcdna3.1 zeo vector (panel C), where the 5′ subcloning primer (SEQ ID NO: 3) includes a Nhe I restriction enzyme site and a Kozak sequence, and the 3′ subcloning primer (SEQ ID NO: 4) includes a Hind III restriction enzyme site and an open reading frame (ORF) stop sequence in accordance with an embodiment of the present invention; cds=coding sequence.

FIG. 2 shows sequences for elastin DNA (SEQ ID NO: 5) (Panel A); protein (SEQ ID NO: 6) (panel A); and primers used to subclone the elastin cDNA into Pcdna3.1 zeo vector (panel B), where the 5′ subcloning primer (SEQ ID NO: 7) includes a Nhe I restriction enzyme site and a Kozak sequence, and the 3′ subcloning primer (SEQ ID NO: 8) includes a Bgl II restriction enzyme site and an ORF stop sequence in accordance with an embodiment of the present invention.

FIG. 3 shows sequences for superoxide dismutase 3 (SOD3) DNA (SEQ ID NO: 9) (panel A); protein (SEQ ID NO: 10) (panel B); and primers used to subclone the SOD3 cDNA into Pedna3.1 zeo vector (panel B), where the 5′ subcloning primer (SEQ ID NO: 11) includes a Nhe I restriction enzyme site and a Kozak sequence, and the 3′ subcloning primer (SEQ ID NO: 12) includes a Hind III restriction enzyme site and an ORF stop sequence in accordance with an embodiment of the present invention.

FIG. 4 shows sequences for TIMP-1 DNA (SEQ ID NO: 13); protein (SEQ ID NO: 14); and primers used to subclone the TIMP-1 cDNA into Pedna3.1 zeo vector, where the 5′ subcloning primer (SEQ ID NO: 15) includes a Nhe I restriction enzyme site and a Kozak sequence, and the 3′ subcloning primer (SEQ ID NO: 16) includes a Hind III restriction enzyme site and an ORF stop sequence in accordance with an embodiment of the present invention.

FIG. 5 shows sequences for COL1A2 DNA (SEQ ID NO: 17) (panels A and B); protein (SEQ ID NO: 18) (panel B); and primers used to subclone the COL1A2 cDNA into Pedna3.1 zeo vector (panel C), where the 5′ subcloning primer (SEQ ID NO: 19) includes a Nhe I restriction enzyme site and a Kozak sequence, and the 3′ subcloning primer (SEQ ID NO: 20) includes a Hind III restriction enzyme site and an ORF stop sequence in accordance with an embodiment of the present invention.

FIG. 6 shows an example of a method used to treat cells having cosmetic function in a subject in accordance with an embodiment of the present invention.

FIG. 7 shows example recombinant DNA molecules in accordance with an embodiment of the present invention.

FIG. 8 shows the cloning strategy for expression of human proteins COLA1A, COL1A2, Elastin, and TIMP-1 in mammalian cells using the CMV promoter-driven eukaryotic vector pcDNA3.1+ zeo:intA in accordance with alternate embodiments of the present invention.

FIG. 9 shows PCR amplification of COLA1A, COL1A2, TIMP-1, and Elastin from normal human tissue cDNAs in accordance with alternate embodiments of the present invention.

FIG. 10 shows transient expression analysis of human COL1A2 in HEK-293T/17 cells in accordance with an embodiment of the present invention. The 138.9 KDa COL1A2 protein is indicated by an arrow.

FIG. 11 shows transient expression analysis of human TIMP-1 in HEK-293T/17 cells in accordance with an embodiment of the present invention. The 23.2 KDa TIMP-1 protein is indicated by an arrow.

FIG. 12 shows transient expression analysis of human Elastin in HEK-293T/17 cells in accordance with an embodiment of the present invention. The 66.1 KDa Elastin protein is indicated by an arrow.

### DETAILED DESCRIPTION

### Definitions

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Also, as used herein, a subject is a mammal who may require treatment to maintain and/or to improve the condition of any cells having cosmetic function. Such subjects may include animals (e.g., animals who may have a skin condition) and humans. The subject may be a human. The subject may comprise a post-menopausal female. Or the subject may be a non-human mammal as for example, pets that may have a need for treatment of the skin, nails, hair, and/or fur.

As used herein, cells that have cosmetic function include skin, fat, muscle, connective tissue, and nerve cells found in the epidermis, dermis and subcutaneous layers, including the nail root or nail bed, nail matrix and nail plate, and scalp, hair follicles and hair strands, as well as muscles found under the subcutaneous fat layer and the tongue; cells in the teeth and gums; cells in the bones, including facial bones; and cells found in the eye including the iris and stroma covering the iris.

As used herein, “epidermal tissue” comprises tissue derived from the ectoderm. The ectoderm is the outermost germ layer of metazoan embryos, developing into epidermal and nervous tissue. Epidermal tissue includes skin, nails, and hair.

As used herein, “skin” is composed of the epidermis and the dermis. The outermost epidermis in skin consists of stratified squamous epithelium with an underlying basement membrane. The epidermis in skin does not contain any blood vessels, but receives nutrients by diffusion from the dermis. The main types of cells that make up the epidermis are keratinocytes. Also, melanocytes and Langerhans cells are present. The epidermis can be further subdivided into the following layers from outermost to innermost: corneum, lucidum, granulosum, spinosum, and basale. The dermis lies below the epidermis and includes blood vessels, nerves, hair follicles, smooth muscle, glands and lymphatic tissue. Also, fibroblasts are commonly found in the dermis and secrete an extracellular matrix rich in collagen, elastin, hyaluronic acid and other macromolecules. Below the dermis lies the subcutaneous layer containing fat-filled cells called adipose cells, larger blood vessels and larger nerves. Muscles are found below the subcutaneous fat. The skin structure is attached via connective tissues to the muscles. Connective tissues also anchor the skin, fat and muscles to underlying bone tissues. Excessive fat in the subcutaneous layer in the attachment areas cause a dimpled “cellulite” appearance.

As used herein, substantially intact cells comprise cells that are not torn, cut or punctured as a result of trauma. The cells may, however, include cells that have been treated with a laser, or by chemical peel, or by dermabrasion or other cosmetic enhancing treatments.

Unless defined otherwise, all technical and scientific terms used herein have the same meaning as commonly understood by one of ordinary skill in the art. Practitioners are particularly directed to Current Protocols in Molecular Biology (Ansubel) for definitions and terms of the art. Abbreviations for amino acid residues are the standard 3-letter and/or 1-letter codes used in the art to refer to one of the 20 common L-amino acids.

The term “recombinant” as used herein in relation to a polynucleotide intends a polynucleotide of semisynthetic, or synthetic origin, or encoded by cDNA or genomic DNA (“gDNA”) such that it is not entirely associated with all or a portion of a polynucleotide with which it is associated in nature.

As used herein, the term “polypeptide” refers to a polymer of amino acids and does not refer to a specific length of the product. Thus, peptides, oligopeptides, and proteins are included within the definition of polypeptide and could be as short as two amino acids. This term also does not exclude post-expression modifications of the polypeptide, for example, glycosylations, acetylations, phosphorylations and the like. Included within the definition are, for example, polypeptides containing one or more analogs of an amino acid (including, for example, unnatural amino acids), polypeptides with substituted linkages, as well as other modifications known in the art, both naturally occurring and non-naturally occurring. As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues. The term “polynucleotide” as used herein refers to a DNA molecule, a RNA molecule or its complementary strand thereof. A polynucleotide molecule can be single or double stranded.

DNA molecules may be identified by their nucleic acid sequences, which are generally presented in the 5′ to 3′ direction, wherein 5′ and 3′ indicate the linkages formed between the 5′-phosphate group of one nucleotide and the 3′-hydroxyl group of the next. For a sequence presented in the 5′-3′ direction, its complement is the DNA strand which hybridizes to that sequence according to the Watson-Crick base pairing model. Thus, the sequence of the complement is defined by the sequence of the original strand, such that adenine base-pairs with thymine, and cytosine base-pairs with guanine.

As used herein, a small inhibitory RNA is a double-stranded RNA of about 20-30 nucleotides than associates with proteins to form an RNAi-induced silencing complex (RISC) that may direct the siRNA to the target RNA sequence. The ds siRNA may then unwind, leaving the antisense strand to signal degradation of the mRNA sequence by endonucleases and exonucleases. In order to obtain lasting therapeutic effects, the RNAi sequence may be expressed long term, preferably under a constitutive promoter. To obtain dsRNA from a vector, it may be expressed as a short hairpin RNA (shRNA), in which there is a sense strand, a hairpin loop region and an antisense strand (Miyagishi et al., J Gene Med 6:715-723, 2004).

As used herein, the term “upstream” refers to a residue that is N-terminal to a second residue where the molecule is a protein, or 5′ to a second residue where the molecule is a nucleic acid. Also as used herein, the term “downstream” refers to a residue that is C-terminal to a second residue where the molecule is a protein, or 3′ to a second residue where the molecule is a nucleic acid. Also, the terms “portion” and “fragment” are used interchangeably to refer to parts of a polypeptide, nucleic acid, or other molecular construct.

The term “vector” refers to a nucleic acid molecule that may be used to transport a second nucleic acid molecule into a cell. In one embodiment, the vector allows for replication of DNA sequences inserted into the vector. The vector may comprise a promoter to enhance and/or maintain expression of the nucleic acid molecule in at least some host cells. Vectors may replicate autonomously (extrachromasomally) or may be integrated into a host cell chromosome. In one embodiment, the vector may comprise an expression vector capable of producing a protein or a nucleic acid derived from at least part of a nucleic acid sequence inserted into the vector.

As is known in the art, conditions for hybridizing nucleic acid sequences to each other can be described as ranging from low to high stringency. Generally, highly stringent hybridization conditions refer to washing hybrids in low salt buffer at high temperatures. Hybridization may be to filter bound DNA using hybridization solutions standard in the art such as 0.5M NaHPO₄, 7% sodium dodecyl sulfate (SDS), at 65° C., and washing in 0.25 M NaHPO₄, 3.5% SDS followed by washing 0.1×SSC/0.1% SDS at a temperature ranging from room temperature to 68° C. depending on the length of the probe (see e.g. Ausubel, F. M. et al., Short Protocols in Molecular Biology, 4^{th} Ed., Chapter 2, John Wiley & Sons, N.Y.). For example, a high stringency wash comprises washing in 6×SSC/0.05% sodium pyrophosphate at 37° C. for a 14 base oligonucleotide probe, or at 48° C. for a 17 base oligonucleotide probe, or at 55° C. for a 20 base oligonucleotide probe, or at 60° C. for a 25 base oligonucleotide probe, or at 65° C. for a nucleotide probe about 250 nucleotides in length. Nucleic acid probes may be labeled with radionucleotides by end-labeling with, for example, [gamma-³²P]ATP, or incorporation of radiolabeled nucleotides such as [alph-³²P]dCTP by random primer labeling. Alternatively, probes may be labeled by incorporation of biotinylated or fluorescein labeled nucleotides, and the probe detected using Streptavidin or anti-fluorescein antibodies.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In one embodiment, the percent identity of two sequences may be determined using GCG with a gap weight of 1, such that each amino acid gap is weighted as if it were a single amino acid mismatch between the two sequences. For example, the term at least 90% identical thereto includes sequences that range from 90 to 99.99% identity to the indicated sequences and includes all ranges in between. Thus, the term at least 90% identical thereto includes sequences that are 91, 91.5, 92, 92.5, 93, 93.5. 94, 94.5, 95, 95.5, 96, 96.5, 97, 97.5, 98, 98.5, 99, 99.5 percent identical to the indicated sequence. Similarly the term “at least 70% identical includes sequences that range from 70 to 99.99% identical, with all ranges in between. The determination of percent identity is determined using the algorithms described here.

As used herein, “homology” refers to the degree of sequence identity between a first sequence and a second sequence (protein or nucleic acid). Typically, the sequence identity between two homologous sequences will be at least 50%. In alternate embodiments, the sequence identity will be no less than 60%; or no less than 75%; or no less than 80%; or at least 90%. In other embodiments, the sequence identity between the two sequences will be at least 95%, or at least 98%, or at least 99%. Also, as used herein, the term “homologue” means a polypeptide having a degree of homology with the wild-type amino acid sequence. Homology comparisons can be conducted by eye, or more usually, with the aid of readily available sequence comparison programs. These commercially available computer programs can calculate percent homology between two or more sequences (e.g. Wilbur, W. J. and Lipman, D. J., 1983, Proc. Natl. Acad. Sci. USA, 80:726-730). For example, homologous sequences may be taken to include an amino acid sequences which in alternate embodiments are at least 75% identical, 85% identical, 90% identical, 95% identical, or 98% identical to each other.

A biologically active or functional derivative (and/or analogue) of any polypeptide includes a polypeptide that has been modified by one or more of an amino acid modification, insertion, deletion, or substitution that does not substantially affect its properties. For example, the biologically active derivative or analog can include conservative amino acid substitutions. Also, a biologically active derivative may comprise a fragment of the native polypeptide. Preferably, the derivative or analog has increased activity or stability compared to native polypeptide. For example, the derivative or analog can include conservative amino acid substitutions. Muteins, analogues and derivatives may be generated using conventional techniques. In an embodiment, the derivative may have increased activity compared to native polypeptide. For example, in certain embodiments, the derivative or analog may comprise at least a 2-fold increase, or at least a 5-10 fold increase, or at least a 20-fold increase.

A “mutein” is polypeptide variant with one or more amino acids altered to produce a desired characteristic, such as to replace a cysteine residue with a non-disulfide bond forming amino acid.

Muteins, analogues and derivatives may be generated using conventional techniques. For example, PCR mutagenesis can be used. While the following discussion refers to DNA, it is understood that the technique also finds application with RNA. An example of a PCR technique is described in WO 92/22653. Another method for making analogs, muteins, and derivatives, is cassette mutagenesis based on the technique described by Wells, Gene, (1985) 34:315.

The terms “analog” or “derivative” in reference to the polypeptides of the present invention also refers to truncations, variants, alleles and derivatives thereof. Where applicable, these terms encompass the bioactivities of “mature” polypeptides or functional isoforms. Thus, polypeptides that are identical or contain at least 60%, preferably 70%, more preferably 80%, and most preferably 90% sequence identity to the mature protein or functional isoform wherever derived, from human or nonhuman sources are included within this definition. The analogs may further include peptides having one or more peptide mimics, also known as peptoids, that possess the bioactivity of the protein. Included within the definition are also polypeptides containing one or more analog amino acid (including, for example, unnatural amino acids, etc.), polypeptides with substituted linkages, as well as other modifications known in the art, both naturally occurring and normaturally occurring. The term polypeptide also does not exclude post-expression modifications of the polypeptide, for example, glycosylations, acetylations, phosphorylations and the like.

Thus, the analogues and/or derivatives of polypeptides used to modulate cells having cosmetic function may contain amino acid substitutions, deletions, or insertions. The amino acid substitutions can be conservative amino acid substitutions or substitutions to eliminate non-essential amino acid residues such as to alter a glycosylation site, a phosphorylation site, an acetylation site, or to minimize misfolding by substitution or deletion of one or more cysteine residues that are not necessary for function.

As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made. Conservative amino acid substitutions are generally those that preserve the general charge, hydrophobicity/hydrophilicity and/or steric bulk of the amino acid substituted, for example, substitutions between the members of the following groups are conservative substitutions: Gly/Ala, Val/Ile/Leu, Asp/Glu, Lys/Arg, Asn/Gln, Ser/Cys/Thr and Phe/Trp/Tyr.

An “expression vector” is a polynucleotide that is operable in a desired host cell and capable of causing the production of a gene of interest in that host cell.

A “regulatory sequence” refers to a polynucleotide sequence that is necessary for regulation of expression of a coding sequence to which the polynucleotide sequence is operably linked. The nature of such regulatory sequences may differ depending upon the host organism. Such regulatory sequences generally include, for example, a promoter, and/or a transcription termination sequence. The term “regulatory sequence” may also include additional components the presence of which are advantageous, for example, a secretory leader sequence for secretion of the polypeptide attached thereto.

“Operably linked” refers to a juxtaposition wherein the components so described are in a relationship permitting them to function in their intended manner. A regulatory sequence is “operably linked” to a coding sequence when it is joined in such a way that expression of the coding sequence is achieved under conditions compatible with the regulatory sequence. Operably linked sequences may have additional nucleotides (or amino acids in a peptide) positioned between the two components of interest.

As used herein, “terminators” are regulatory sequences, such as polyadenylation and transcription termination sequences, located 3′ or downstream of the stop codon of the coding sequences.

As used herein, “recombinant host cells,” “host cells,” “cells,” “cell cultures,” and other such terms denote, for example, microorganisms, insect cells, and mammalian cells, that can be or have been used as recipients for introduction of recombinant vector or other transfer DNA, and include the progeny of the cell that has been transformed.

“Transformation” or “transfection,” as used herein, refers to the transfer of an exogenous polynucleotide into a host cell, irrespective of the method used for the transfer, which can be, for example, by infection, direct uptake, transduction, F-mating, injection, microinjection or electroporation. The exogenous polynucleotide may be maintained as a non-integrated vector, for example, in some cases, a plasmid, or alternatively, may be integrated into the host genome.

“Purified” and “isolated” in reference to a polypeptide or a nucleotide sequence means that the indicated molecule is present in substantial absence of other biological macromolecules of the same species or type. In alternate embodiments, the term “purified” as used herein refers to at least 75% by weight; or at least 85% by weight, or at least 95% by weight or at least 98% by weight, of biological macromolecules of the same type.

A “pharmaceutically acceptable carrier,” is any carrier that is used by persons in the art for administration into a human that does not itself induce any undesirable side effects such as the production of antibodies, fever, etc.

The term “treating” or “treat” refers to improving, or preventing the worsening of a condition (e.g., wrinkles in the skin), and may comprise curing the condition, substantially preventing the onset of the condition, or improving the subject's condition. In certain embodiments, the term “treatment” may refer to a full spectrum of treatments for a given condition relating to cosmetic appearance for which the subject may have, including alleviation of one symptom or most of the symptoms resulting from that condition, a cure for the particular condition, or prevention of the onset of the condition.

An “effective amount,” as used herein refers to that amount that is effective for production of a desired result. This amount varies, for example, depending upon the health and physical condition of the individual to be treated, the capacity of the individual's immune system to synthesize antibodies, the degree of protection desired, the formulation, the attending physician's assessment of the medical situation, and other relevant factors.

As used herein, “in vivo transfection” or “in vivo incorporation” refers to the process where the biomolecule of interest is introduced into a cell in a living body. The term includes transfection of naked polynucleotides, or polynucleotides that include an additional moiety or carrier into cells. Methods used for in vivo transfection are described in detail herein.

As used herein, “ex vivo transfection” or “ex vivo incorporation” refers to the process where the biomolecule of interest is introduced into a cell that is outside of a living body. The term includes transfection of naked polynucleotides, or polynucleotides that include an additional moiety or carrier into cells. Methods used for ex vivo transfection are described in detail herein.

As used herein, a polypeptide involved in maintaining cells having cosmetic function may be any peptide as described herein that can improve the production of biomolecules that can enhance the health or longevity of such cells. As is known in the art, the peptide and gene sequences for such proteins are available on public databases.

As used herein, the term “platelet derived growth factor” or “PDGF” includes the PDGF A chain polypeptide and the PDGF B chain polypeptide and to the AA, BB, and AB dimers, and biologically active fragments, analogs, and derivatives thereof as described in U.S. Pat. No. 5,187,263; Waterfield et al., Nature 304:35-39 (1983); Wang et al., J. Biol. Chem. 259: 10645-48(1984), Antoniades et al., Biochem. Pharm. 33: 2833-38(1984); and Westermark et al., Proc. Natl. Acad. Sci. USA 83:7197-7200(1986); U.S. Pat. No. 5,219,759. A polypeptide “having the biological activity of PDGF” refers to a polypeptide having the same or increased capability of preferentially stimulating the growth of cells of the dermis layer of the skin. Such a polypeptide can be a full-length PDGF, a fragment of PDGF, an analog of PDGF bearing amino acid substitution, deletion or addition or a derivative of PDGF, such as that described in U.S. Pat. No. 5,149,792 and EP 458 959 B1; and U.S. Pat. Nos. 4,769,328; 4,801,542; 4,766,073; 4,849,407; 4,845,075; 4,889,919; 5,045,633; and 5,128,321.

As used herein, the term “keratinocyte growth factor” or “KGF” refers to a member of a group of structurally distinct proteins known as FGFs that display varying degrees of sequence homology, suggesting that they are encoded by a related family of genes. The FGFs share common receptor sites on cell surfaces. KGF, for example, can bind to FGFR-3. The term “keratinocyte growth factor” or “KGF” refers to any one of a mature polypeptide and biologically active fragments, analogs, and derivatives thereof as described in, for example, WO 90/08771 and WO 95/01434. A polypeptide “having the biological activity of KGF” refers to a polypeptide having the same or increased capability of preferentially stimulating the growth of cells of the epidermis layer of the skin. Such a polypeptide can be a full-length KGF, a fragment of KGF, an analog of KGF bearing an amino acid substitution, deletion, or addition; or a derivative of KGF as described in WO 90/08771, and WO 95/01434.

The term “insulin-like growth factor” as used herein encompasses IGF-I and IGF-II in their substantially purified, native, recombinantly produced, or chemically synthesized forms, and includes biologically active fragments, analogues, muteins, including C-terminal deletion muteins, and derivatives thereof that retain IGF activity and/or ability to bind the IGF receptors, as described in, for example, EP 135 094, WO 85/00831, U.S. Pat. No. 4,738,921, WO 92/04363, U.S. Pat. No. 5,158,875, EP 123 228, and EP 128 733. Also included are IGF-I and IGF-II in their substantially purified, native, recombinantly produced, or chemically synthesized forms, and includes biologically active fragments, analogues, muteins, including C-terminal deletion muteins, and derivatives thereof that retain IGF activity and/or ability to bind the IGF receptors, as described in, for example, EP 135 094, WO 85/00831, U.S. Pat. No. 4,738,921, WO 92/04363, U.S. Pat. No. 5,158,875, EP 123 228, and EP 128 733. A polypeptide “having the biological activity of IGF” refers to a polypeptide having the same or increased capability of acting as growth factor capable of insulin-like effects such as, for example, stimulation of phosphorylation of specific tyrosine residues within the cytoplasmic domain of the receptor to which it binds as described in WO 93/98826, or, in some cases, for example, mitogenic effects on certain cells as described in EP 0 128 733. Such a polypeptide can be full-length IGF, a fragment of IGF, an analog of IGF bearing an amino acid substitution, deletion, or addition, or any derivative of IGF.

As used herein, the term “insulin like growth factor binding protein (IGFBP)” refers to a binding protein identified to bind an IGF binding protein as described and identified in Keifer et al, J. Biol. Chem. 266: 9043-9 (1991), Camacho-Hubner et al, J. Biol. Chem. 267: 11949-56 (1992), McCusker and Clemens, THE INSULIN LIKE GROWTH FACTORS: STRUCTURE AND BIOLOGICAL FUNCTIONS, Oxford Univ. Press, N.Y. pp. 110-150 (1992). A polypeptide “having the biological activity of IGFBP” refers to a polypeptide having about the same or an increased capability of acting as an IGF binding protein by binding and transporting IGF to tissue and cells where IGF- can have a biological effect. As there are presently at least six IGFBPs known, many of which significantly different from the other IGFBPs, specific qualities regarding the biological activity of a given IGFBP does not include necessarily the entire group of IGF binding proteins. Thus, the biological activity of an IGFBP may have some similarities to other IGFBPs, but may also have distinctions that identify it as a unique IGFBP.

As used herein, “full-length PDGF” or “mature PDGF” and “full-length KGF” or “mature KGF” and “full-length IGF” or “mature IGF”, and “full length IGFBP” and “mature IGFBP” refers to the respective native polypeptide as found in human or other mammalian tissues.

As used herein, the term “collagen” refers to a member of a large group of at least 28 structurally related proteins (Collagen I-XXVIII) that display varying degrees of sequence homology, suggesting that they are encoded by a related family of genes. Collagens are the main fibrous polypeptides composing extracellular matrices and structural support of the skin, including the dermis and dermal-epidermal junction, and other body tissues. These proteins form a variety of structurally and functionally important supramolecular assemblies. (See e.g., Pihlajamaa T., New tools for the study of an old collagen characterization of the human COL9A1, COL9A2 and COL9A3 genes and production of human type IX collagen as a recombinant protein. Collagen Research Unit, Biocenter Oulu and Department of Medical Biochemistry, University of Oulu, FIN-90220 Oulu, Finland, 2000, Oulu, Finland). Collagens are characterized by a triple helix consisting of three identical or different polypeptides, called alpha chains. Collagen is characterized by division into two major structural groups i.e. fibrillar and non-fibrillar. The group of fibrillar, or fibril-forming, collagens consists of types I, II, III, V and XI.

Collagen types I, II and III are referred to as the major fibrillar collagens, implying abundance in a number of tissues. Type I collagen is expressed in most connective tissues and it is the most abundant type of all, being the major structural component of skin, bone, tendon and ligaments. Type I collagen is mostly present in the form of heterotrimers of two al(I) chains and one α2(I) chain, encoded by the COL1A1 and COL1A2 genes, respectively. Type II is the major collagenous component of cartilage, the vitreous humour and the intervertebral disc, and is also detected in the inner ear and transiently in numerous other tissues during development. It is a homotrimer of three α1(II) chains encoded by the COL2A1 gene. Type III collagen is also a homotrimer, consisting of α1(III) chains. It is expressed in most tissues that contain type I collagen, but not in bone or tendon. Type III is an abundant component of elastic tissues, including the skin, blood vessels, gut and lung, and can be assembled into heterotypic fibrils with type I collagen. Instead of forming fibrils, the non-fibrillar collagens serve various other functions. Characteristically their triple helix is divided into several segments on account of noncollagenous interruptions. The non-fibrillar collagens may be divided into six subgroups in terms of their structure or function. Important network-forming collagens include types IV, VI, and VII. Type IV collagen networks are present in all basement membranes at numerous locations in the body. In most locations this supporting and controlling network contains α1 (IV) and α2(IV) chains, but certain basement membranes, e.g. glomerular membranes, also include α3(IV), α4(IV), α5(IV) and α6(IV) chains. The collagenous region of these α chains is about 1400 amino acids long with numerous short interruptions. The genes encoding these polypeptides form an interesting exception among the generally randomly located collagen genes as they are located pairwise on three chromosomes in a head-to-head fashion. Type VI collagen is a heterotrimer of α1(VI), α2(VI) and α3(VI) chains consisting of a short triple helix flanked by large globular N and C-terminal domains. It is the only collagen aggregating into beaded microfilaments, which are to be found on the cell surface and around collagen fibres in most connective tissues and may serve to anchor the cells to the macromolecular framework of the ECM. Type VII collagen forms anchoring fibrils upon dimerization and lateral association of homotrimeric α1(VII)3 molecules. These fibrils link the epithelial basement membrane to the underlying ECM in skin, cornea and several other epithelial tissues. The highly interrupted triple helix of type VII is the longest among all the collagens, and the gene COL7A1 encoding it has the highest number of exons of the known genes, i.e. 108. Such collagen polypeptides can be full-length collagen, a fragment of collagen, or a functional analog of collagen. Table 1 includes many of the types of collagen that are important to cosmetic cell function and that may be used in the methods and compositions of the present invention.

TABLE 1
Types of Collagen

| Type | Activities in Cell | Gene(s) |
|---|---|---|
| I | This is the most abundant collagen of the human body. It is | COL1A1, COL1A2 |
| | present in scar tissue, the end product when tissue heals by repair. | |
| | It is found in tendons, the endomysium of myofibrils and the | |
| | organic part of bone. | |
| | Involved in osteogenesis imperfecta, Ehlers-Danlos Syndrome | |
| II | Hyaline cartilage, makes up 50% of all cartilage protein | COL2A1 |
| III | This is the collagen of granulation tissue, and is produced quickly | COL3A1 |
| | by young fibroblasts before the tougher type I collagen is | |
| | synthesized. Reticular fiber. Also found in artery walls, intestines | |
| | and the uterus. Involved in Ehlers-Danlos Syndrome | |
| IV | basal lamina; eye lens. Also serves as part of the filtration system | COL4A1, COL4A2, |
| | in capillaries and the glomeruli of nephron in the kidney. Involved | COL4A3, COL4A4, |
| | in Alport syndrome | COL4A5, COL4A6 |
| V | most interstitial tissue, assoc. with type I, associated with placenta | COL5A1, COL5A2, |
| | | COL5A3 |
| VI | most interstitial tissue, assoc. with type I. Involved in Ulrich | COL6A1, COL6A2, |
| | myopathy and Bethlem myopathy. | COL6A3 |
| VII | forms anchoring fibrils in dermal epidermal junctions, involved in | COL7A1 |
| | epidermolysis bullosa | |
| VIII | some endothelial cells | COL8A1, COL8A2 |
| IX | FACIT collagen, cartilage, assoc. with type II and XI fibrils | COL9A1, COL9A2, |
| | | COL9A3 |
| X | hypertrophic and mineralizing cartilage | COL10A1 |
| XI | cartilage | COL11A1, |
| | | COL11A2 |
| XII | FACIT collagen, interacts with type I containing fibrils, decorin | COL12A1 |
| | and glucosaminoglycans | |
| XIII | transmembrane collagen, interacts with integrin a1b1, fibronectin | COL13A1 |
| | and components of basment membranes like nidogen and | |
| | perlecan. | |
| XIV | FACIT collagen | COL14A1 |
| XV | — | COL15A1 |
| XVI | — | COL16A1 |
| XVII | Transmembrane collagen, also known as BP180, a 180 kDa | COL17A1 |
| | protein. Involved in Bullous Pemphigoid and certain forms of | |
| | junctional epidermolysis bullosa | |
| XVIII | source of endostatin | COL18A1 |
| XIX | FACIT collagen | COL19A1 |
| XX | — | COL20A1 |
| XXI | FACIT collagen | COL21A1 |
| XXII | — | COL22A1 |
| XXIII | — | COL23A1 |
| XXIV | — | COL24A1 |
| XXV | — | COL25A1 |
| XXVII | — | COL27A1 |
| XXVIII | — | COL28A1 |

As used herein, the term “elastin” refers to a tropoelastin or mature elastin polypeptide. Elastin is a key extracellular matrix protein that is critical to the elasticity and resilience of many vertebrate tissues including skin large arteries, lung, ligament, tendon, skin, and elastic cartilage (see Mithieux and Weiss, 2005, Elastin, Advances in Protein Chemistry, Vol 70, p 437). The human gene encoding tropoelastin is a single copy localized to 7q11.2 region. The primary transcript is approximately 40 kb in length and contains small exons interspersed between large introns giving rise to an unusually low exon/intron ratio. This sequence codes for an mRNA of ˜3.5 kb, which consists of a ˜2.2 kb coding segment and a relatively large, 1.3 kb 30 untranslated region. The human tropoelastin gene contains 34 exons. Tropoelastin is distinguished by an exon periodicity where functionally distinct hydrophobic and crosslinking domains are encoded in separate alternating exons. All the exons exist as multiples of three nucleotides and the exon-intron borders are always split in the same way. The first nucleotide of a codon is found at the 30 junction, while the second and third nucleotides are present at the 50 border of exons. The primary transcript of tropoelastin undergoes extensive alternative splicing. As the splitting of codons at the exon-intron borders is consistent throughout the molecule, alternative splicing occurs in a cassette-like fashion with maintenance of the coding sequence. This results in the translation of multiple heterogeneous tropoelastin isoforms. At least seven human exons are known to be alternatively spliced: 22, 23, 24, 26A, 30, 32, and 33. Alternative splicing of individual exons may be used to tailor the structural function of the protein in different tissues. It appears to be developmentally regulated and tissue-specific with age-related changes in isoform ratios observed in all species that have been investigated. The most frequently observed human tropoelastin isoform lacks exon 26A, which is reportedly only expressed in certain disease states. Three human disorders have been linked to mutations or deletions of the tropoelastin gene: cutis laxa, supravalvular aortic stenosis, and Williams-Beuren syndrome.

As used herein, the term “TIMP” refers to a member of a group of at least 4 structurally related proteins (Tissue inhibitor of matrix metalloproteinase-1, -2, -3 and -4 (TIMP-1, TIMP-2, TIMP-3 and TIMP-4)) that display varying degrees of sequence homology, suggesting that they are encoded by a related family of genes. TIMPs are critical to limiting extracellular matrix breakdown by inhibiting collagenases, gelatinases, and the like (matrix metalloproteinases (MMPs)). An essential feature of all TIMPs is that they have 12 conserved cysteine residues, with conserved relative spacing, and the presence of a 23 to 29 amino acid leader sequence, which is cleaved to produce a mature protein. Crystal structures for TIMPs, and MMP-TIMP complexes such as TIMP-1 in complex with MMP-3 and TIMP-2 with MT1-MMP have been described. TIMPs have the shape of an elongated, contiguous wedge consisting of the N-terminal and the C-terminal halves of the polypeptide chains opposing each other (Gomis-Ruth et al. 1997). In complexes with MMPs, TIMPs bind with their edge into the entire length of the active-site cleft of MMPs.

TIMP-1 protein is a 184 amino acid glycoprotein with a molecular mass of 28.5 kDa. It contains two possible N-glycosylation sites. The TIMP-1 promoter contains 10 Sp1, six AP-1, six PEA3, 12 AP-2 sites and five CCAAT boxes, in addition to a putative binding site for the transcription factor leader-binding protein 1 (LBP-1). The upstream TIMP-1 element-1 (UTE-1) is also essential for TIMP-1 transcription. The promoter contains two novel repressive elements, and an unidentified Ets-related factor to suppress transcription (Dean et al. 2000). TIMP-1 protein has been detected in human dentin and cementum. In addition, human osteoblasts secrete TIMP-1 constitutively.

TIMP-2 is a nonglycosylated 194 amino acid protein of 21 kDa molecular mass. It has an extended negatively charged C-terminus. The TIMP-2 promoter contains several regulatory elements including five Sp1, two AP-2, one AP-1 and three PEA-3 binding sites. TIMP-2 is transcribed into two mRNAs of 1.2 and 3.8 kb. Human osteoblasts and chondrocytes secrete TIMP-2.

The TIMP-3 polypeptide sequence is 37% and 42% similar to the sequences of TIMP-1 and TIMP-2, respectively. The TIMP-3 protein has 188 amino acids. It has a conserved glycosylation site near the C-terminus. Characterisation of the human recombinant TIMP-3 reveals that it has both a 27 kDa glycosylated and a 24 kDa unglycosylated species. TIMP-3 is localised to the ECM in both its glycosylated and unglycosylated forms. The TIMP-3 gene has four Sp1 sites, but no TATA-box in the promoter. Three TIMP-3 mRNA species of 2.4, 2.8 and 5.5 kb are transcribed from the gene, and are constitutively expressed by human chondrocytes.

TIMP-4 is a 195 amino acid polypeptide with molecular mass of 22 kDa. The TIMP-4 polypeptide is 37% identical to TIMP-1 and 51% identical to TIMP-2 and -3. TIMP-4 is the most neutral TIMP protein under physiological conditions (pH 7.4), having an isoelectric point of 7.34, compared with values of 8.00, 6.45 and 9.04 for human TIMP-1, TIMP-2 and TIMP-3, respectively. The TIMP-4 gene is transcribed into 1.4 kb mRNA species. Of the calcified tissues, TIMP-4 has been detected in human cartilage.

Each TIMP binds with both a different rate of interaction and affinity to a target MMP, usually in 1:1 or 2:2 stoichiometrical fashions. TIMP-1 inhibits MMP-1, MMP-3 and MMP-9 more effectively than TIMP-2. TIMP-2 inhibits proMMP-2 over 10-fold more effectively than TIMP-1. However, TIMP-2 has a bi-functional effect on MMP-2 since MT-MMP mediated proMMP-2 activation requires a tiny amount of TIMP-2 to make activation progress, whereas a greater concentration of TIMP-2 inhibits MMP-2. TIMP-3 inhibits at least MMP-2 and MMP-9, whereas TIMP-4 is a good inhibitor for all classes of MMPs without remarkable preference for specific MMPs. TIMP-4 regulates MMP-2 activity both by inhibiting MT1-MMP and by inhibiting activated MMP-2.

While TIMPs usually inhibit already active MMP, gelatin-binding MMPs are an exception, since TIMP reversibly binds to the proforms of both MMP-2 and MMP-9. Later, TIMP may be dissociated from the complex, and proMMP activation is allowed to proceed. For example, TIMP-1 binds to the proMMP-9, and further proMMP-9 activation by MMP-3 is prevented until TIMP-1 is inactivated in the complex, e.g. by neutrophil elastase, which does not destruct proMMP-9. However, TIMP-2 may also inhibit the active form of MMP-9. Active MMP-13 is an example of an MMP, which is inhibited by all types of TIMPs, and MMP-19 is inhibited by all TIMPs, except TIMP-1. The activity of soluble MMP-16 is inhibited by TIMP-2 and TIMP-3, but not TIMP-1.

As used herein, “MMPs” comprise a family of at least 28 secreted or transmembrane enzymes collectively capable of processing and degrading various ECM proteins. Of these, at least 22 MMPs have so far been found to be expressed in human tissues. MMPs share high protein sequence homology and have defined domain structures and thus, according to their structural properties, MMPs are classified either as secreted MMPs or membrane anchored MMPs, which are further divided into eight discrete subgroups. Secreted MMPs include minimal-domain MMPs, simple hemopexin domain-containing MMPs, gelatin-binding MMPs, furin-activated secreted MMPs and vitronectin-like insert MMPs, while membrane bound MMPs include type I transmembrane MMPs, glycosyl-phosphatidyl inositol (GPI)-linked MMPs and type II transmembrane MMPs.

Crystal structures of MMPs further uncovered the exact domain organization, polypeptide fold and main specificity determinants. To date, crystal structures of the catalytic domains of human MMP-1, MMP-2, MMP-3, MMP-7, MMP-8, MMP-9, MMP-11, MMP-12, MMP-13 and MMP-14, in addition to porcine full length MMP-1 and human proMMP-2 have been resolved.

The third level of restricting the proteolytic activities of MMPs includes endogenous tissue inhibitors of MMPs (TIMPs). TIMPs specifically inhibit active forms of MMPs, and in some cases, latent MMPs as well, and disturbance in this balance may lead to pathological situations in tissues. Active MMPs may also be inactivated by α-macroglobulins, particularly α2-macroglobulin. Recent findings indicate that serine proteinase inibitor, tissue factor pathway inhibitor-2 (TFPI-2), inhibits MMP-1, MMP-2, MMP-9 and MMP-13 (Herman et al. 2001). Calcium-binding proteoglycans (N-Tes, testican-1 or testican-3) are able to inhibit MT1- or MT3-MMP mediated proMMP-2 activation. In addition, there are many exogenous inhibitors. Some examples include flavonols in green tea such as epigallocatechin-3-gallate or catechins, which inhibit MMP-2, MMP-9, MMP-12 activities and proMMP-2 activation. Several synthetic inhibitors are also good inhibitors of MMPs activities. A polypeptide “having the biological activity of TIMP” refers to a polypeptide having the same or increased capability of preferentially inhibiting MMPs. Such a polypeptide can be a full-length TIMP, a fragment of TIMP, an analog of TIMP bearing an amino acid substitution, deletion, or addition; or a derivative of TIMP.

As used herein, the term “elafin” refers to a mature elafin polypeptide (see e.g., Francart, et. al., 1997, Solution Structure of R-elafin, a Specific Inhibitor of Elastase, J. Mol. Biol. (1997) 268, 666-677). A polypeptide “having the biological activity of elafin” refers to a polypeptide having the same or increased capability of preferentially inhibiting elastase. Such a polypeptide can be a full-length elafin, a fragment of elafin, an analog of elafin bearing an amino acid substitution, deletion, or addition; or a derivative of elafin as described in U.S. Pat. No. 5,734,014. Elafin is also commonly called “skin-derived antileukoproteinase; elastase inhibitor; and SKALP). Elafin is a 57 amino acid residue peptide (6 kDa) that was first isolated from scales of patients with psoriasis. Elafin is a specific inhibitor of human leukocyte elastase (HLE) and porcine pancreatic elastase, and of proteinase-3, three enzymes that possess the ability to cleave the important connective tissue protein elastin. It has no inhibition effect on other serine proteinases such as trypsin, plasmin, a chymotrypsin, and cathepsin G. The binding of elafin to its target is very tight with dissociation constants. Elafin is believed to play an important protective role against destructive degradation by excessive elastase of the structural integrity of elastin-containing tissues and is therefore a compound of potentially therapeutical value in elastase-mediated disorders. Recently, elafin has been shown to protect elastin from UV-induced elastolytic degradation by physically binding to elastin fibers. Elafin gene sequences are publicly available.

As used herein, the term “superoxide dismutase” or “SOD” refers to a member of a group of at least 3 structurally related enzymatic proteins (SOD 1-3) that display varying degrees of sequence homology, suggesting that they are encoded by a related family of genes. Superoxide dismutase catalyzes the dismutation of superoxide into oxygen and hydrogen peroxide. As such, it is an important antioxidant defense in nearly all cells exposed to oxygen. In humans, three forms of superoxide dismutase are present. SOD1 is located in the cytoplasm, SOD2 in the mitochondria and SOD3 is extracellular. The first is a dimer (consists of two units), while the others are tetramers (four subunits). SOD1 and SOD3 contain copper and zinc, while SOD2 has manganese in its reactive centre. The genes are located on chromosomes 21, 6 and 4, respectively (21q22.1, 6q25.3 and 4p15.3-p15.1). SOD has been shown to prevent telomere shortening in fibroblasts, convert myofibroblasts into fibroblasts, and reduce fibrotic scarring in skin post-irradiation (Serra, et. al., 2003. J Biol Chem. February 28; 278(9):6824-30; Vozenin-Brotons. et. al., 2001. Free Radic Biol Med. January 1; 30(1):30-42). The SOD 1 and SOD 2 gene sequences are publicly available.

As used herein, the term “heat shock proteins” or “HSPs” refers to a member of a large group of structurally related proteins that display varying degrees of sequence homology, suggesting that they are encoded by a related family of genes. Heat shock proteins (Hsps) are highly conserved constitutive and induced proteins that may be found in cells from bacteria to human beings (S. Lindquist, Ann. Rev. Biochem 55, 1151 (1986)). Constitutive Hsps can be critical to many diverse cellular functions. There are six major groups of Hsps that are grouped based upon molecular size. The groups are: (a) 20-30 kDa; (b) 40-50 kDa; (c) 50-60 kDa; (d) 70 kDa; (e) 90 kDa; and (f) 100-110 kDa (Minowada G et al., J. Clin. Invest., 95, 3 (1995); Morris S D, Clin. Exper. Dermatol., 27, 220 (2001)). The inducible forms of Hsps may be elicited by a variety of stressors, including elevated temperature (M. Schlesinger et al., Cold Spring Harbor Laboratory, 1982), heavy metals (M. Schlesinger et al., Alan R. Liss, Inc., 137 (1989)), amino acid analogs (P. Kelley and M. Schlesinger, Cell 15, 1277 (1978); L. Hightower, J. Cell. Physiol. 102, 407 (1980)), oxidative radicals (M. Ashburner, Chromosoma 31, 356 (1970); J. Compton and B. McCarthy, Cell 14, 191 (1978)), ischemia or return from anoxia (S. Guttman, Cell 22, 299 (1980); M. Ashburner and J. Bonner, Cell 17, 241 (1979)), mechanical trauma (L. Hightower and F. White, Cold Spring Harbor Laboratory, 369 (1982); D. Gower et al., J. Cell Biol. 103, 291 (1986)), and the presence of abnormal proteins in the cell (J. Ananthan et al., Science 232, 522 (1986)). The unifying functional characteristic of heat shock proteins is to act to maintain normal cellular function under non-ideal conditions.

Hsps may be expressed constitutively in normal or resting skin cells where they may play an important role in several important biological processes. For example, Hsps in skin may act as molecular chaperones (Maytin E V, J. Invest. Dermatol., 104, 448 (1995)), and/or to effect the rapid upregulation of stress proteins in response to environmental stressors (Welch W J, Physiol. Rev., 72, 1063 (1992)). Several Hsps are expressed in skin cells. Hsp 72 is constitutively expressed in keratinocytes, (see e.g., Trautinger F et al., J. Invest. Dermatol., 101, 334 (1993); Charveron M et al., Cell Biol. Toxicol., 11, 161 (1995); and Laplante A et al., J. Histochem. Cytochem., 46, 1291 (1998)). Also, Hsps 27, 47, 60, 90, and 110 may be present in normal epidermis (Wilson N et al., J. Cutan. Pathol., 27, 176 (2000)). Each of these Hsps appear to have specific functions within normal tissue. For example, Hsp 27 stabilizes actin, whereas Hsp 90 acts both as a molecular chaperone, and/or may activate certain transcription factors (Charveron M et al., Cell Biol. Toxicol., 11, 161 (1995)). Hsp 27 in skin tissue may also control differentiation of cells in developing skin (Jantschitsch C et al., Br. J. Dermatol., 139, 247 (1998)).

There is evidence that Hsps can be exchanged between cells (L. Hightower and P. Guidon, J. Cell. Physiol. 138, 257 (1989); M. Tytell et al., Brain Res. 363, 161 (1986); M. Tytell, Int. J. Hyperhermia, 21, 4450455 (2005)). Thus, it is possible that the effects of Hsps may not be limited to the cell in which the Hsp is expressed, but may extend to neighboring cells.

### Genetic Modification of Cells Having Cosmetic Function

Thus, the present invention relates to methods and compositions for genetic modification of cells having cosmetic function to enhance cosmetic appearance. The present invention may be embodied in a variety of ways.

In one embodiment, the invention may comprise a method for the cosmetic genetic modification of substantially intact cells having a cosmetic function in a subject comprising: administering a polynucleotide encoding at least one of a nucleic acid or a polypeptide involved in maintaining the cells having cosmetic function to a least a portion of the cells such that the nucleic acid or polypeptide is expressed in the cells having cosmetic function to enhance and/or maintain a biochemical and/or physiological process that has a positive effect on cosmetic appearance.

In another embodiment, the present invention comprises an isolated polynucleotide encoding at least one of a nucleic acid or a polypeptide involved in maintaining the cells having cosmetic function. In an embodiment, the isolated nucleotide is applied to cells, such that the nucleic acid or polypeptide may be expressed in the cells having cosmetic function to enhance and/or maintain a biochemical and/or physiological process that has a positive effect on cosmetic appearance.

In another embodiment, the present invention comprises an isolated polypeptide involved in maintaining the cells having cosmetic function.

In yet other embodiments, the present invention may comprise a composition for genetically modifying substantially intact cells having cosmetic function in a subject. The composition may comprise a polynucleotide encoding at least one of a nucleic acid or a polypeptide involved in maintaining cells having cosmetic function; and a carrier for administration of the polynucleotide to a least a portion of the subject's cells having cosmetic function such that the nucleic acid or polypeptide is expressed in the cells having cosmetic function so as to enhance and/or maintain a biochemical and/or physiological process that has a positive effect on cosmetic appearance. Or, the composition may comprise a polypeptide involved in maintaining cells having cosmetic function; and a carrier for administration of the polynucleotide to a least a portion of the subject's cells having cosmetic function such that the nucleic acid or polypeptide is expressed in the cells having cosmetic function so as to enhance and/or maintain a biochemical and/or physiological process that has a positive effect on cosmetic appearance.

A variety of polypeptides may be targeted to enhance and/or maintain a biochemical and/or physiological process that has a positive effect on cosmetic appearance. For example, in an embodiment, the polynucleotide encodes an collagen polypeptide or polypeptides (e.g., COL1A1 and COL1A2). Or, the polynucleotide may encode an elastin polypeptide. In other embodiments, the polynucleotide may encode a TIMP-1 polypeptide. Or, the polynucleotide may encode a SOD polypeptide. In yet other embodiments, the polypeptide may comprise at least one of a keratinocyte growth factor, a transforming growth factor, a platelet derived growth factor, a vascular endothelial growth factor, an insulin-like growth factor, a heptocyte growth factor, a vascular endothelial growth factor, a fibroblast growth factor, an epidermal growth factor, a platelet derived endothelial cell growth factor, a connective tissue growth factor, a granulocyte-macrophage colony-stimulating factor, a macrophage colony stimulating factor, a growth hormone, TSP-1, TSP-2, a collagen protein, TIMP-1, a superoxide dismutase, an elastin, or a hypoxia inducible factor, or a biologically active derivative thereof.

Thus, in alternate embodiments of the methods and compositions of the present invention, the polynucleotide may comprise SEQ ID NO: 1, SEQ ID NO: 5, SEQ ID NO: 9, SEQ ID NO: 13, or SEQ ID NO: 15, or the complement of SEQ ID NO: 1, SEQ ID NO: 5, SEQ ID NO: 9, SEQ ID NO: 13, or SEQ ID NO: 17, or a sequence that is at least 70%, 85%, 80%, 90%, 95%, 96%, 97%, 98%, or 99% identical to SEQ ID NO: 1, SEQ ID NO: 5, SEQ ID NO: 9, SEQ ID NO: 13, or SEQ ID NO: 15, or the complement of SEQ ID NO: 1, SEQ ID NO: 5, SEQ ID NO: 9, SEQ ID NO: 13, or SEQ ID NO: 17.

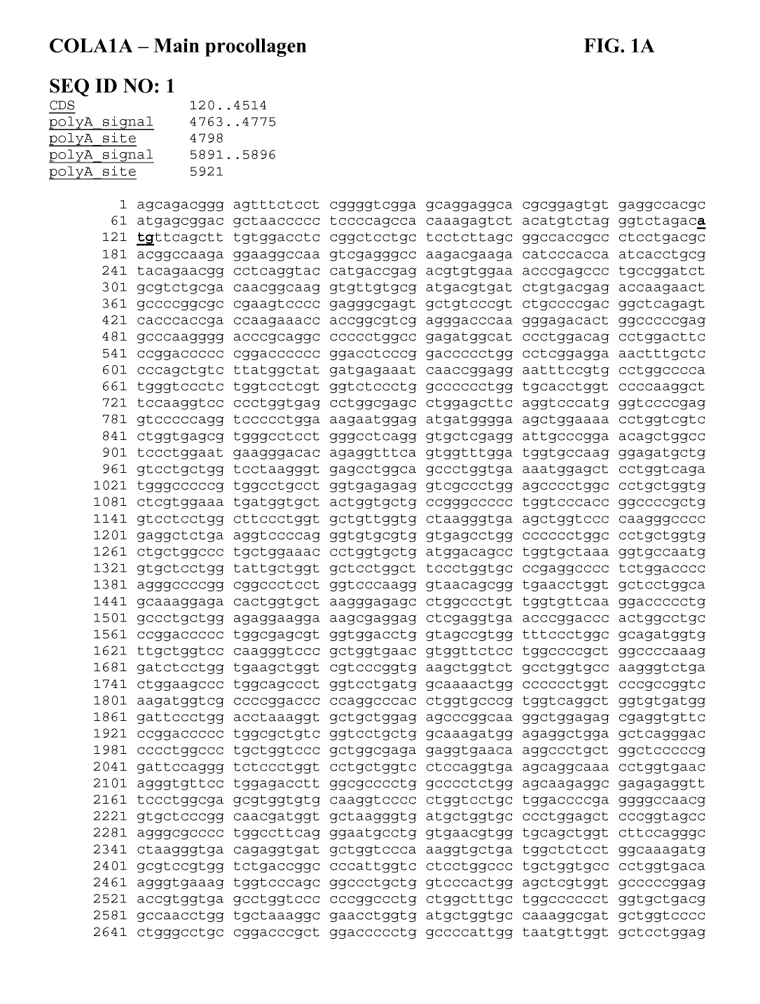

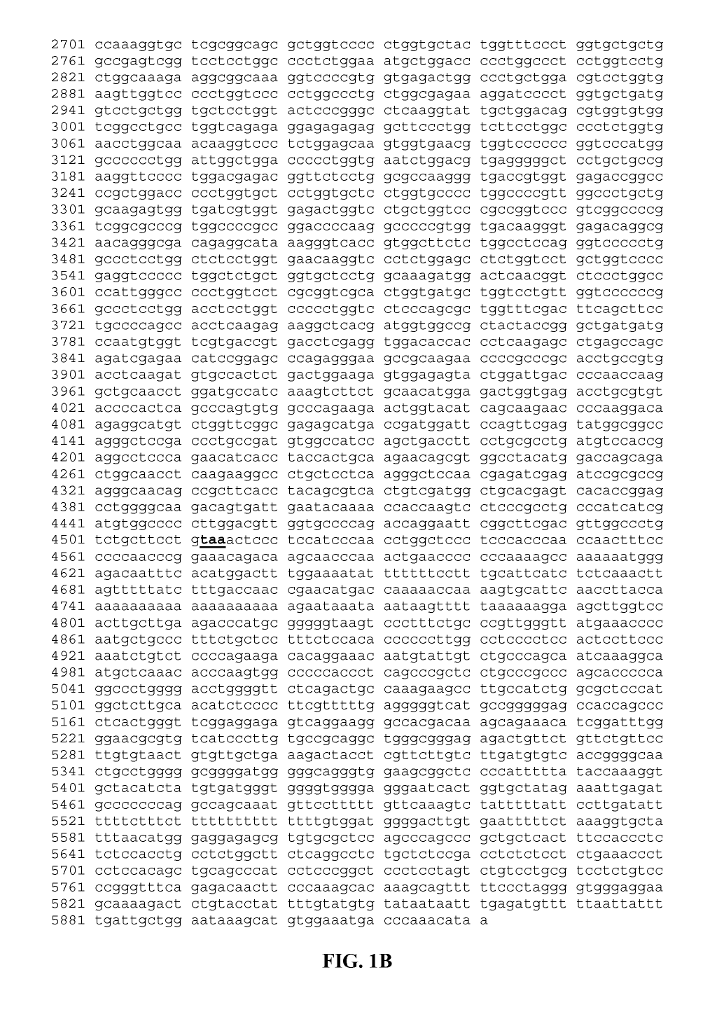

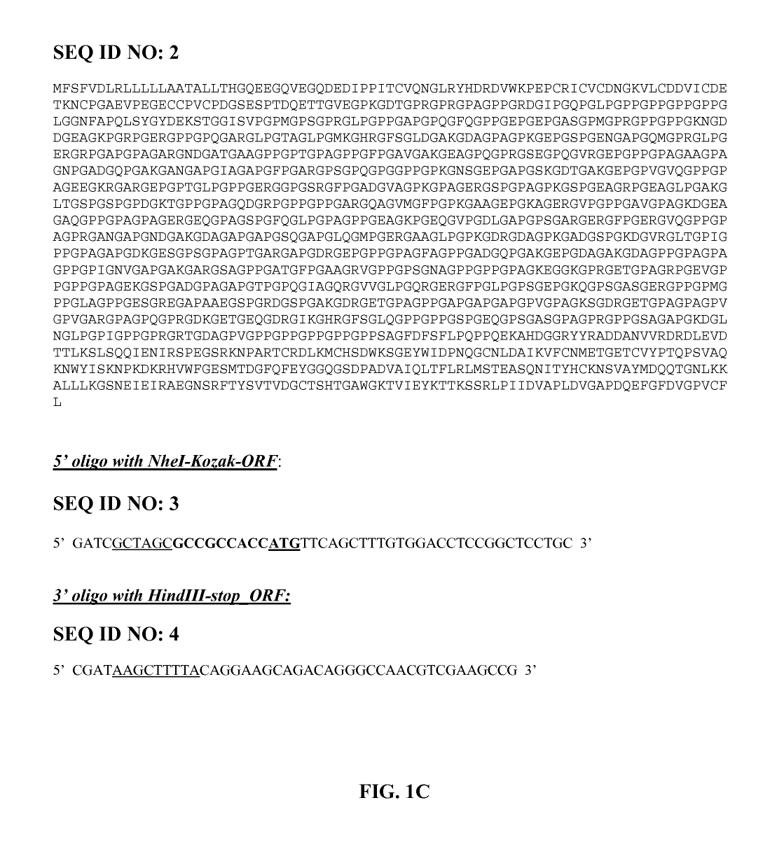

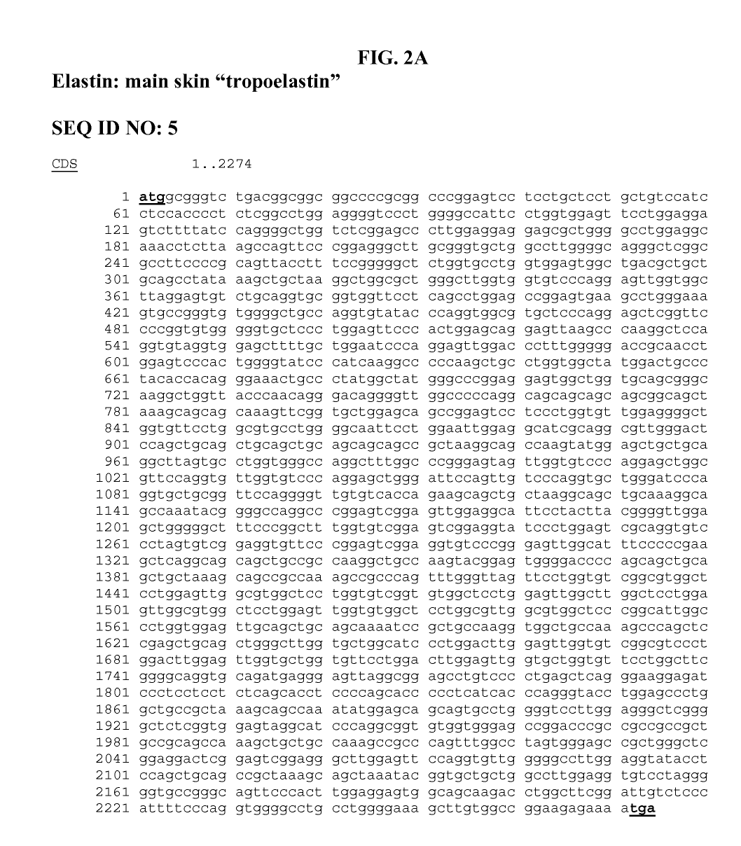

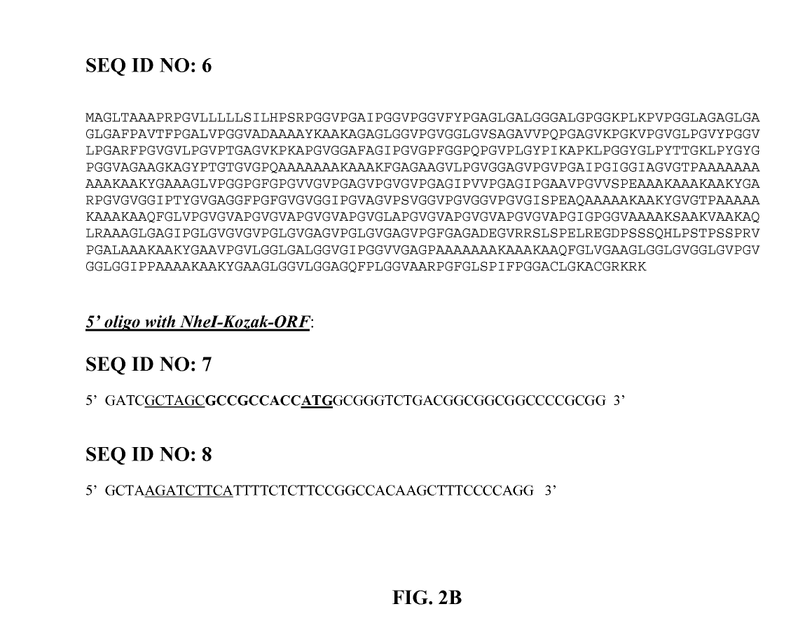

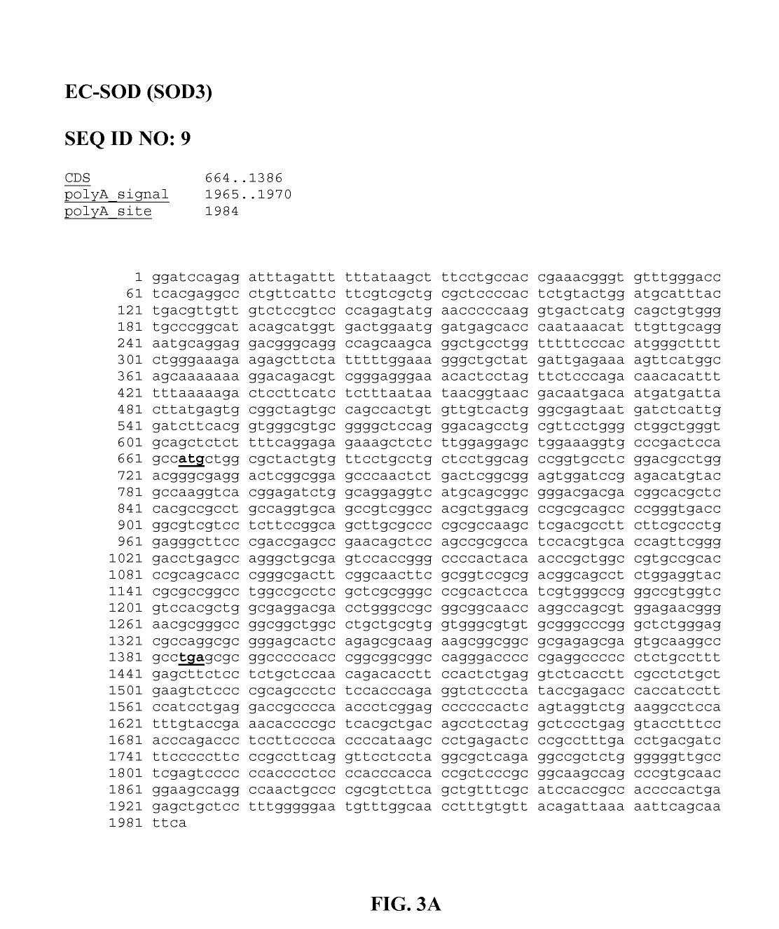

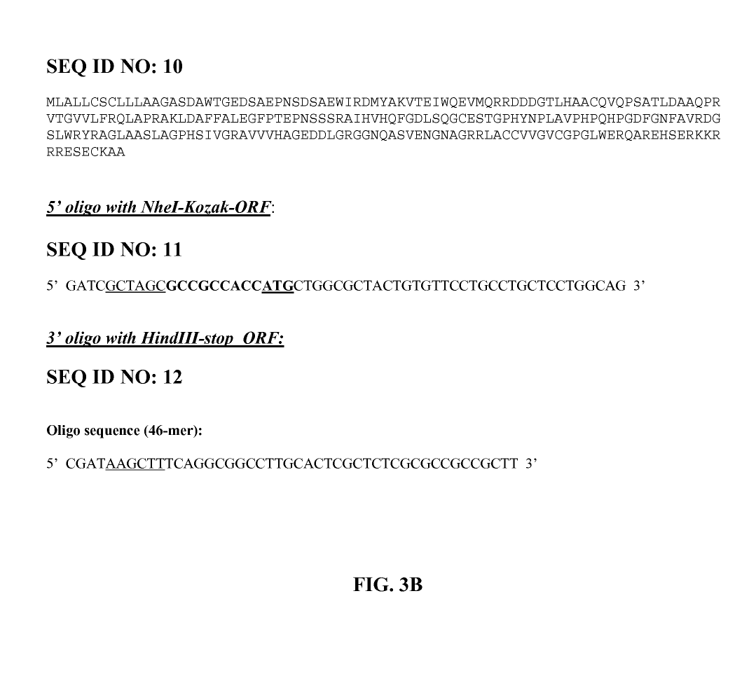

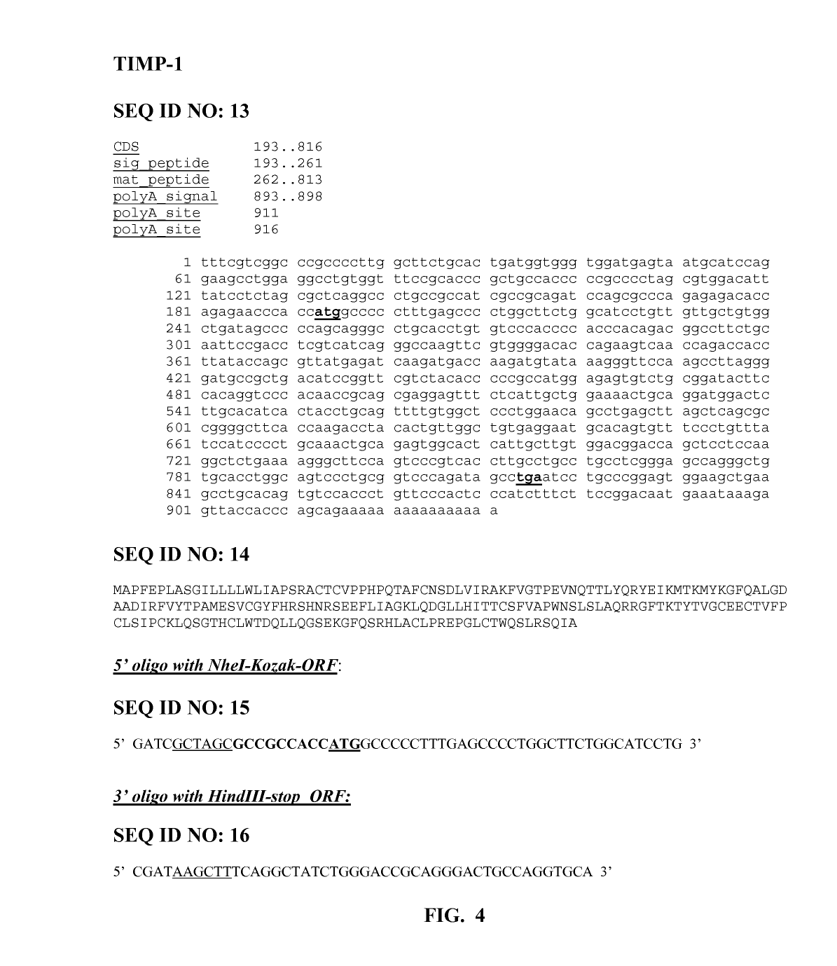

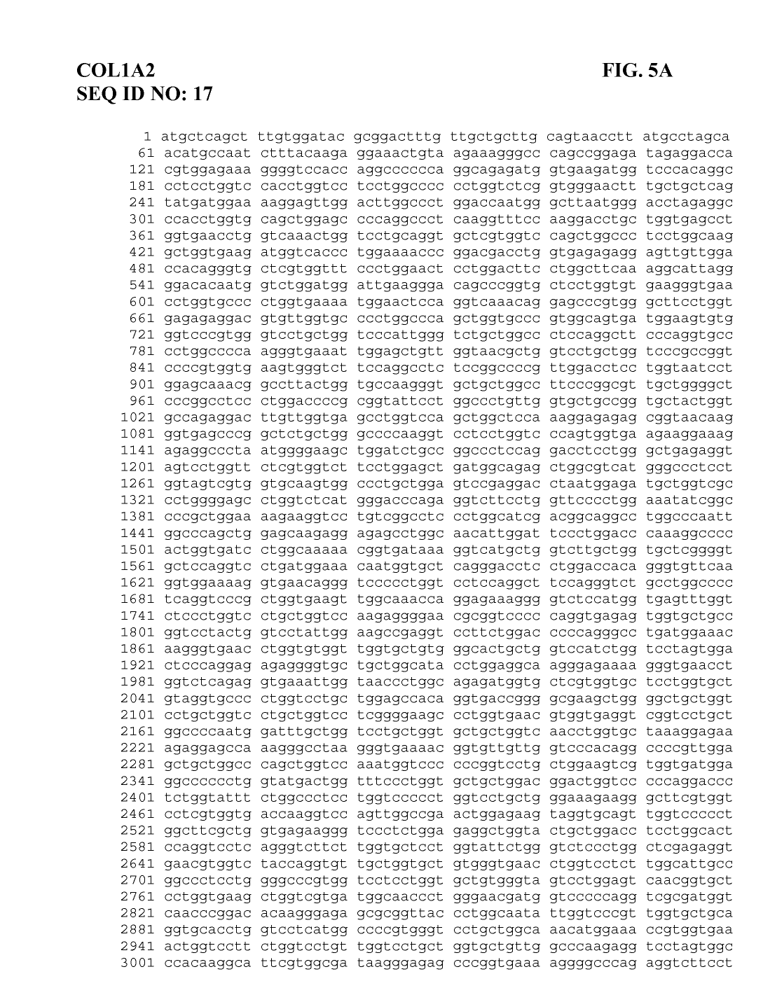

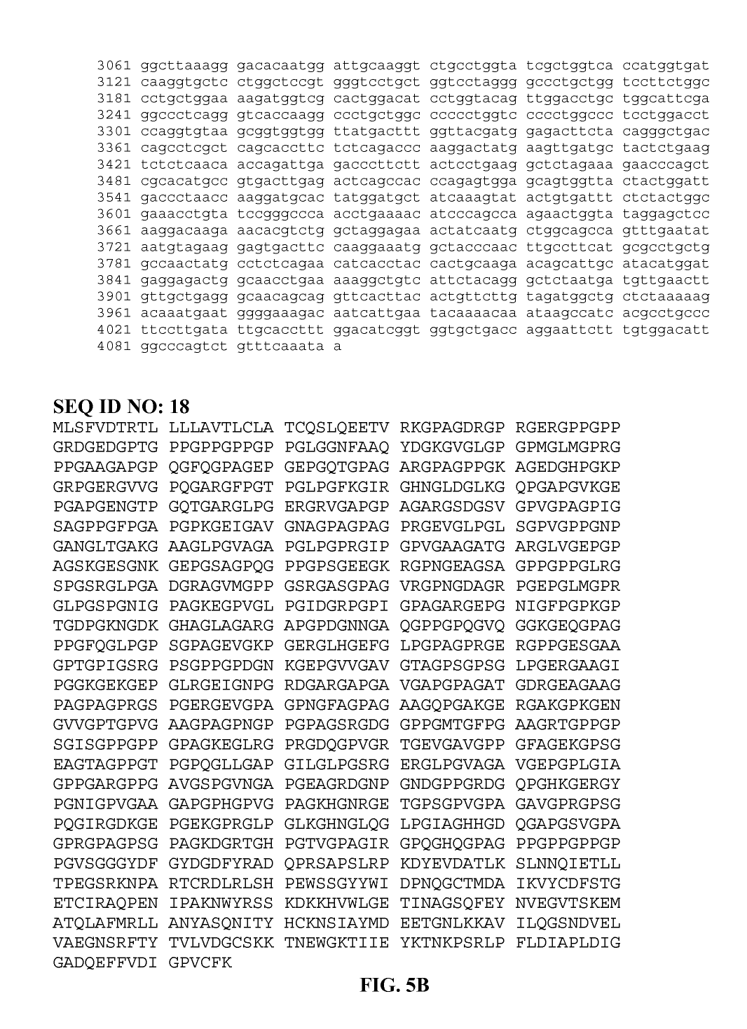

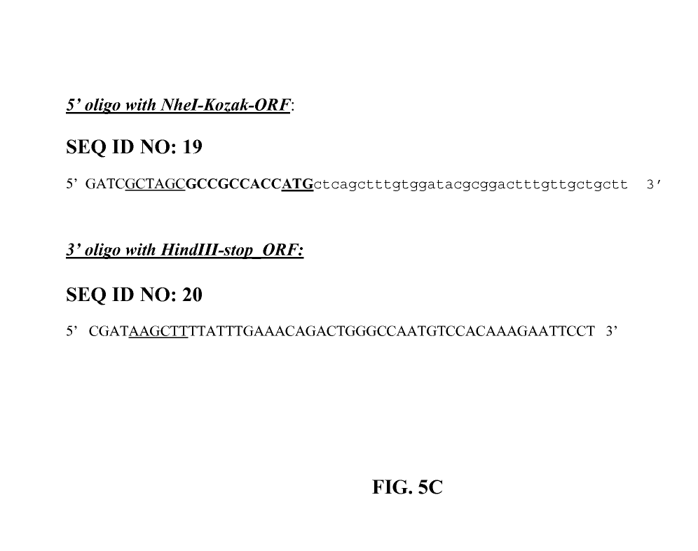

In other embodiments, the polynucleotide may encode for a polypeptide of SEQ ID NO: 2 (procollagen COL1A1), SEQ ID NO: 6 (elastin), SEQ ID NO: 10 (superoxide dismutase 3), SEQ ID NO: 14 (TIMP-1) or SEQ ID NO: 18 (COL1A2), or a sequence that is at least 70%, 85%, 80%, 90%, 95%, 96%, 97%, 98%, or 99% identical to a polypeptide of SEQ ID NO: 2, SEQ ID NO: 6, SEQ ID NO: 10, SEQ ID NO: 14 or SEQ ID NO: 18. The sequences of these nucleotides and polypeptides are shown in FIGS. 1-5.

In an embodiment, the polynucleotide comprises a plasmid or a viral vector. For example, the polynucleotide may maintained as a extrachromosomal plasmid in at least a portion of the transfected cells.

The methods and compositions of the present invention may comprise delivery of a single agent or multiple agents to cells. In one embodiment, at least two different polypeptides or nucleic acid molecules are administered to the cells.

The polynucleotide may comprise a recombinant construct that is designed to express the polypeptide in cells having cosmetic function. Thus, as described in more detail herein, the polynucleotide encoding a nucleic acid or polypeptide involved in maintaining the cells having a cosmetic function may be operably linked to a constitutive or an inducible promoter. In an embodiment, the promoter is not ubiquitously expressed, but is expressed in the cells having cosmetic function. Additionally or alternatively, the polynucleotide encoding a nucleic acid or polypeptide involved in maintaining the cells having a cosmetic function may be operably linked to an enhancer. Also, in yet other embodiments, the polynucleotide encoding a nucleic acid or polypeptide involved in maintaining the cells having a cosmetic function may be operably linked to at least one of a functional poly A sequence, an intron, a cleavage sequence, a stop sequence, or a cap site.

A variety of cells having a cosmetic function may be targeted using the methods and compositions of the present invention. In alternate embodiments, the modified cells having a cosmetic function comprise at least one of keratinocytes, fibroblasts, adipocytes, or myofibrils.

A variety of methods may be used to administer the nucleic acid (i.e., polynucleotide) constructs of the present invention. In one embodiment, the polynucleotide is introduced as naked DNA into the cells having a cosmetic function. In alternate embodiments, the polynucleotide is introduced into the cells having a cosmetic function via at least one of liposomes, nanoparticles, an emulsion, a thixogel, or an organoleptic gel. Or, the polynucleotide may be introduced into the cells having a cosmetic function via a water-in-oil emulsion or an oil-in-water emulsion. In yet other embodiments, the polynucleotide is introduced into the cells having a cosmetic function via at least one of particle mediated transfer (e.g., gold particles, microspheres), voltage driven transfer, radio frequency ablation-mediated transfer, ultrasound, or microneedles.

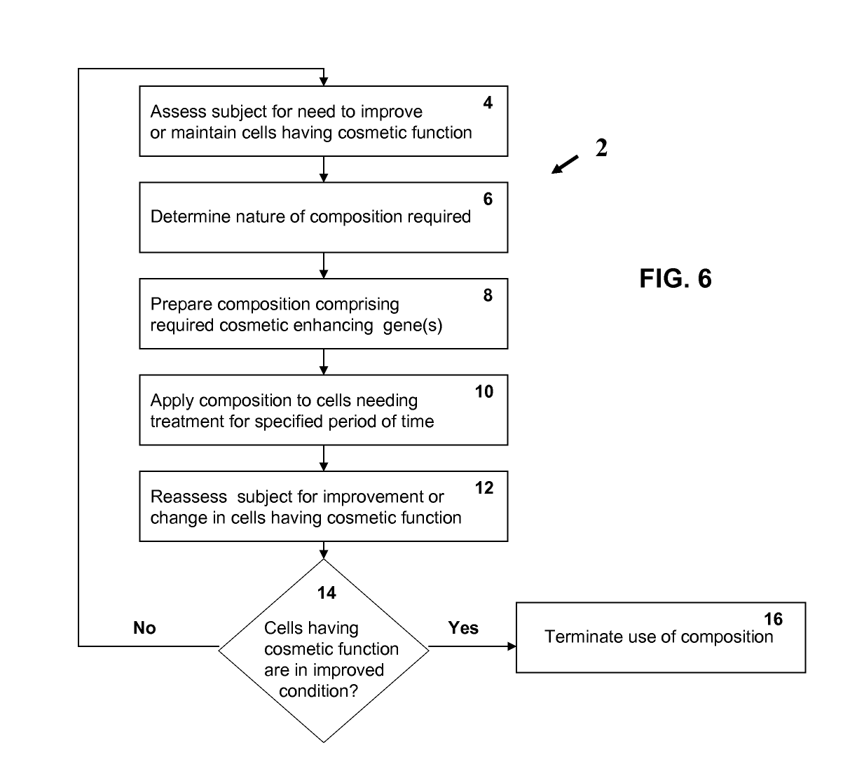

FIG. 6 shows an embodiment of an example method 2 of the present invention. For example, the method may comprise the step 4 of assessing the subject for the need to improve or maintain cells having cosmetic function. The assessment may be done by a dermatologist or another health care professional. Or, the assessment may be performed by the subject (i.e., a self-assessment).

Next, the method may comprise the step 6 of determining the nature of the treatment that is required. Again, the assessment may be done by a dermatologist or another health care professional. Or, the assessment may be performed by the subject (i.e., a self-assessment).

The method may further comprise the step 8 of preparing (or selecting) a composition comprising the required therapeutic gene or mixture thereof. In an embodiment, this step may be performed at least initially by a dermatologist or another health care professional. Once the appropriate composition has been selected, an assessment may be performed by the subject (i.e., a self-assessment). For example, the subject may perform an assessment as to which of the available skin care genetic products to purchase.

The method may additionally comprise the step 10 of applying the composition to the cells or tissue needing treatment. In an embodiment, the composition may be applied for a period of time as is required to improve and/or maintain the cells having cosmetic function. As described in more detail herein, the exact dosage and time period of treatment may vary depending upon the condition of the subject and the nature of the composition applied.

The method may also comprise the step 12 of reassessing the subject to determine if there has been an improvement in the subject's cells having cosmetic function. If it is determined that the cells having cosmetic function have an improved appearance 14 the subject may choose to terminate the use of the composition 16. If, however, it is determined that the cells having cosmetic function do not have an improved appearance 14 the subject may choose to repeat the procedure, perhaps using a different composition.

Thus, embodiments of the present invention comprise methods and compositions for the genetic modification of cells having cosmetic function. In certain embodiments, the present invention may comprise methods and/or compositions for the in vivo or ex vivo transfection of recombinant nucleic acid constructs into cells having cosmetic function. The recombinant constructs may be incorporated into cells having cosmetic function either as extrachromosomal elements or may insert directly into the genome.

In other embodiments, the methods and/or compositions may provide polypeptides or other biomolecules directly (i.e, without the need for expression of the gene). For example, a mixture of growth factors that may enhance or maintain cells that have cosmetic function may be used. For example, in alternate embodiments, the polynucleotides may comprise keratinocyte growth factor (KGF), insulin-like growth factor 1 (IGF-I), and/or transforming growth factor beta (TGF-beta), and/or mixtures of these peptides as discussed in detail herein. Or, other peptides may be used.

Or, the recombinant constructs may encode for regulatory polynucleotides, as for example antisense RNA or inhibitory RNAs (RNAi) to enhance and/or maintain a biochemical and/or physiological process that has a positive effect on cosmetic appearance.

The methods and or compositions of the present invention may be utilized to enhance and/or maintain and/or improve the cosmetic appearance of a subject and/or a subject's tissue and/or cells having a cosmetic function. A variety of biological molecules and/or physiological processes may be modulated using the methods and/or compositions of the present invention. In certain embodiments, the cosmetic benefits provided by the methods and compositions of the present invention may include at least one of the following: reduced skin sagging; increased skin thickness; increased skin volume; reduced wrinkle number; reduced wrinkle length, reduced wrinkle depth; increased skin tightness, firmness, tone, or elasticity; increased skin hydration and ability to retain moisture, water flow (e.g. aquaporine channel production) and osmotic balance.

Modulation of at least one of a variety of biomolecules having expression in cells that have cosmetic function may result upon treatment with the compositions and/or methods of the present invention. For example, use of the methods and compositions of the present invention may also, in certain embodiments, result in increased skin proteins. Or, use of the methods and compositions of the present invention may result in increased levels of skin lipids, such as membrane lipids, lamellar body lipids, secreted intercellular lipids. Thus, in certain embodiments, use of the methods and/or compositions of the present invention may result in increased extracellular matrix, and/or adhesion and communication polypeptides, including but not limited to collagens, elastins, keratins (including keratin intermediate filaments), fibronectins, proteoglycans, laminins, integrins, decorin, lumican, fibromodulin (and other are small leucine-rich repeat proteoglycans (SLRP's)), Tenascin E, neurofilaments, nestins, desmins, vimentins, peripherins, ceramides, cholesterol, phospholipids, sphingolipids, “Natural Moisturizing Factor” (NMF), and glycosaminoglycans (GAGs) such as hyaluronic acid and dermatan sulfate.

In yet other embodiments, use of the methods and/or compositions of the present invention may result in increased skin cell energy production, utilization and conservation; improved oxygen utilization; improved skin cell life (e.g. longer living fibroblasts) and/or life cycle (e.g. reduced senescence). Embodiments of use of the methods and/or compositions of the present invention may additionally or alternatively result in at least one of improved skin cell immunity defense, heat shock/stress response, antioxidant defense capacity to neutralize free radicals (e.g. reactive oxygen or carbonyl species), and/or toxic defense (e.g. environmental pollutants); improved protection and recovery from ultraviolet rays. In yet other embodiments, use of the methods and/or compositions of the present invention may result in improved skin cell communication (e.g. neuropeptide mediated communication) and skin cell innervation; improved skin cell cohesion/adhesion (e.g. desmosome integrity); improved calcium mineral and other mineral metabolism; improved skin cell turnover (e.g. desquamation); and/or improved skin cell circadian rhythms.

Use of the method and compositions of the present invention may, in certain embodiments result in at least one of improved skin texture, smoothness, softness radiance, glow. For example, in certain embodiments, use of the method and/or compositions of the present invention may result in at least one of reduced discolorations and unevenness of skin color including redness, hyperpigmentation (e.g., melasma) and hypopigmentation.

Yet other embodiments of the use of the methods and compositions of the present invention may result in at least one of improved blood vessel health including improved vascular integrity (i.e. less leakage of discoloring blood products into skin), improved vascular tone, improved breakdown of discoloring blood by-products (e.g. improved hemosidirin breakdown, bilirubin processing and improved UGT1a enzyme function) and reduced “spider veins” and “varicose veins.” In yet other embodiments, use of the methods and compositions of the present invention may result in improved DNA, RNA, mitochondrial, membrane and other skin cell organelle health; improved adipogenesis to increase fat amounts to keep skin plumped and the face or other body areas looking full, defined or rounded; improved dermal-epidermal junction (“DEJ). For example, one of the most evident and reproducible biological feature of aging skin is the flattening of the dermal-epidermal junction. This process may occur as a consequence of a rarification and reduction of dermal papillae. For example, between the age of 30 and 90 years, a more than 50% decrease in the interdigitation between these skin layers may take place (see e.g., Kirstin et al., Phytochemistry and Photobiology, 2005, 81:581-587).

There may be certain biomolecules that may be over-expressed in cells having cosmetic function. Thus, in yet other embodiments, use of the methods and/or compositions of the present invention may be used to reduce such detrimental biomolecules. For example, embodiments of the methods and/or compositions of the present invention may result in increased lipolysis to reduce “cellulite” and “dimpling” of skin; as well as to reduce abdominal and/or total body fat. Or, the methods and/or compositions of the present invention may also be used to result in reduced pore size; reduced dryness and/or flaking; reduced oiliness and acne. Embodiments of the methods and/or compositions of the present invention may also be used to result in decreased contraction of muscles, smooth muscle cells and myofibrils (e.g. to reduce expression wrinkles), or increased contraction of muscles, smooth muscle cells and myofibrils (e.g. to reduce sagging); and increased size of skin cells (e.g. hypertrophied facial muscles to prevent a “hollowed” face look). Additionally, cells having cosmetic function (e.g., skin cells) may be modified for the purpose of maintaining cosmetic appearance despite UV light damage (e.g. enhanced endonuclease, photolyase, heat shock proteins, metallothionein, and superoxide dismutase expression levels for an increased endogenous “sunscreen” to protect cosmetic appearance) and other environmental aggressions (e.g. pollutants and irritants).

Yet other embodiments of the methods and/or compositions of the present invention may be used to increase pigmentation (e.g. sun tan without UV exposure for a sunless tan) or alternatively, decrease pigmentation (e.g. “whitening”). Additionally, skin cells may be modified for the purpose of producing permanent or temporary color and pigmentation changes (e.g. changing color of hair or iris) including fluorescence, iridescence, phosphorescence, reflectance, refraction, photoluminescence, chemiluminescence, and/or bioluminescence (e.g. luciferase and luciferin reaction).

Additionally, cosmetic improvement and cosmetic maintenance benefits upon treatment with the methods and/or compositions of the present invention may include at least one of the following: reduced scar formation post-trauma; improved post-laser resurfacing repair of the skin; increased protection from sun-sensitizing drugs such as ACCUTANE®, anti-depressants, RETIN-A MICRO®, and the like; reduced photo-aging damage; improved cosmetic appearance and healing after laser resurfacing/pulsed light therapy or any similar method that uses light, laser, radio waves, electromagnetic, ultrasound waves and the like to treat skin cells; improved cosmetic appearance and healing post-dermabrasion procedures, post-chemical peels (large variety of chemical peels); scar reduction (proactive and retroactive), and post-injectable procedures including BOTOX®, Restylane, Juviderm and the like.

Although the methods and/or compositions of the present invention may, in certain embodiments, be used on skin, other types of cells having cosmetic function may also be targeted. For example, nail-related cells may be modified for improved nails and cuticles with faster growth rate, reduced splitting and breaking, improved length and thickness, decreased ridging and flaking, decreased roughness and dullness, and better coloration. Also, hair-related cells may be modified for faster growth rate, lower growth rate, reduced split ends, flaking and breaking, improved manageability, reduced dullness, improved shine and sheen, increased density (thicker), decreased density (thinner), ablation, increased length and thickness and improved softness. Any one of the above cosmetic improvements may produce the secondary benefit of greater self-confidence and enhanced mood in a subject.

Thus, in certain embodiments, the invention provides in vivo or ex vivo methods for increasing expression of cosmetic enhancing and/or maintaining nucleic acids or polypeptides in cells having cosmetic function. For example, the method may comprise administering a polynucleotide encoding at least one of a nucleic acid or a polypeptide involved in maintaining the cells having cosmetic function to a least a portion of the cells such that the nucleic acid or polypeptide is expressed in the cells having cosmetic function to enhance and/or maintain a biochemical and/or physiological process that has a positive effect on cosmetic appearance.

A variety of genes that encode for a variety of proteins may be used in the methods and compositions of the present invention. In certain embodiments, the polynucleotide construct may encode for an antisense RNA or a siRNA as a means to inhibit expression of the protein. For example, reduced expression of certain growth factors may result in reduced inflammation in or near the cell having cosmetic function. Also, reduced expression of certain MMP proteins may result in increased collagen levels in the cells.

In an embodiment, the nucleic acid, or polypeptide(s) encoded by the transfected recombinant polynucleotide, may comprise a keratinocyte growth factor (e.g., KFG-1 or KFG-2), and/or KGF receptors and cofactors or a fragment thereof. Alternatively or additionally, the nucleic acid, or polypeptide(s) encoded by the transfected recombinant polynucleotide, may comprise at least one a transforming growth factor (TFG-alpha, TFG-beta₁₋₄, other known TGF-beta proteins) and/or TGF receptors or cofactors or a fragment thereof. Alternatively or additionally, the nucleic acid, or polypeptide(s) encoded by the transfected recombinant polynucleotide, may comprise an insulin-like growth factor (e.g., IGF-1, IGF-2) or a fragment thereof. Alternatively or additionally, the nucleic acid, or polypeptide(s) encoded by the transfected recombinant polynucleotide, may comprise at least one of a platelet derived growth factors (e.g., PDGF-AA, PDGF-AB, PDGF-BB, PDGF-CC) or a fragment thereof.

In other embodiments, the nucleic acid, or polypeptide(s) encoded by the transfected recombinant polynucleotide, may comprise at least one of the following polypeptides or fragments thereof: a vascular endothelial growth factor (e.g. VEGF-A or VEGF-C); a hepatocyte growth factor; a fibroblast growth factor (e.g., FGF-1, FGF-2, FGF-3, or FGF-22); an epidermal growth factor (e.g., EGF; Heparin-binding-EGF); a platelet derived endothelial cell growth factor (PD-ECGF); a connective tissue growth factor (e.g., CTGF-1, CTGF-2); a granulocyte-macrophage colony-stimulating factor (GMCSF); a monocyte colony stimulating factor (MCSF); a granulocyte colony-stimulating factor (GCSF); a growth hormone (GH); an anti-angiogenic factors (e.g. Ang-1, soluble platelet factor-4, thrombospondins such as TSP-1 and TSP-2, an antagonist of urokinase plasminogen activator/receptor, e.g., uPA/uPAR, angiostatin, endostatin and vasostatin); a transcription factor Egr-1, or a hypoxia inducible factor (HIF-1-alpha).

Other encoded polypeptides or nucleic acids may be used in the compositions and methods of the present invention may comprise polypeptides having enzymatic function for synthesis, degradation, repair and antioxidant protection, polypeptides with structural function including extracellular matrix proteins, polypeptides with cellular adhesion and communication function, factors, cofactors, receptors for the expressed polypeptides, and promoters for non-transfected genomic polynucleotides encoding polypeptides active in skin cells. For example, the polypeptides or nucleic acids used in the compositions and methods of the present invention may comprise, or encode (or be complementary to a nucleotide that encodes) for polypeptides or fragments thereof of neurotrophins (e.g. nerve growth factors (NGFs), neurotrophin-3 (NT-3), NT-4 and brain-derived neurotrophic factor (BDNF), tumor necrosis factors (TNFs), hepatocyte growth factors (HGFsi), interferons (INFs), interleukins (ILs—e.g. IL-1, IL-2, IL6, IL-8 and IL-10), angiopoietins, scatter factors (SFs), chemokines (CCs), activins (part of TGF-beta family), adipokines (e.g. leptin, interleukin 6 (IL-6), other cytokines, adiponectin, complement components, adipsin, plasminogen activator inhibitor-1 (PAI-1), proteins of the renin-angiotensin system (RAS) and others), bone morphogenetic proteins (BMPs).

In yet other embodiments, the polynucleotide may encode (or be complementary to a nucleotide that encodes) for a CCN protein family peptide (e.g. cysteine-rich (CYR61/CNN1), a connective tissue growth factor CNN2, November, WISP-1, WISP-2, and WISP-3), integrin alpha(6)beta(1), cell surface heparan sulfate proteoglycans (HSPGs), focal adhesion kinase, paxillin, and/or Rac, p42/p44 MAPKs, or a fragment or biologically active derivative thereof. Or, siRNAs or antisense molecules to these genes may be used.

In yet other embodiments, the polynucleotide may encode (or be complementary to a nucleotide that encodes) at least one of the following polypeptides (or biologically active derivative or fragments thereof): a signal transducer and/or activators of transcription (STATs), a thyroid hormone (TH), a thyroid stimulating hormone (TSH), prolactin, parathyroid hormone (PTH), a Parathyroid hormone-related protein (PTHrP), a thyrotropin releasing hormone (TRH), neuropeptides, (e.g. endorphin) tazorac induced genes (TIGs), cellular retinoic acid-binding proteins (CRABPs), procollagen, collagen (all formes), proline hydroxylase and other enzymes involved in collagen synthesis (e.g. hydroxylysyl galactosyltransferase and galactosylhydroxylysyl glucosyltransferase), repair/maintenance and degradation, tropoelastin, elastin, lysyl oxidase and other enzymes involved in elastin synthesis, repair/maintenance and degradation, fibrillins, fibulins, superoxide dismutase (e.g., SOD 1, 2 and 3), glutathione peroxidase (GSH-Px), catalase (CAT), peroxiredoxin VI, haem-oxygenase (HO), thioprotein reductase, antioxidant protein 2 (Aop2), metallothionein (MT), glutathione (GSH), ubiquinol (coenzyme Q), calnexin, enzymes involved in keratinocyte desquamation (e.g. cathepsins such as Cathepsin-D and Cathepsin-G and transglutaminases), fibronectins, laminins, integrin, cadherins, lectin cell adhesion molecules (LEC-CAMs), aquaporins, actins, involucrins, loricrins, (pro)filaggrin, keratin/cytokeratins, desmoplakins, envoplakins, periplakins, annexins, enzymes involved in the synthesis, maintenance/repair and degradation of extracellular matrix, cellular adhesion and cellular communications (e.g. enzymes involved in the synthesis of laminin, fibronectin and keratins), DNA synthesis, repair/maintenance and degradation enzymes (e.g., endonucleases, photolyase, telomerase, and others (ERCC3, PCNA, RPA, XPA, p53, all of which are decreased with age; Goukassian et al., FASEB J., 2000, 14, 1325-1334) and telomere 3-prime overhang sequence (T-oligos), stress response and chaperone proteins and related elements/activators/factors (e.g. heat shock protein 27, heat shock protein 47, heat shock protein 60, heat shock protein 70, alphaB-crystalline, Grp78, Grp94, heat shock element (HSE) by heat shock transcription factors and co-factors (e.g. HSF1, 2, 4), cysteine string protein (csp), BAG-1, Hip, CHIP, Hop and Tpr-2), enzymes involved in synthesis, repair/maintenance and degradation of ceramides and ceramide derivatives (e.g. serine palmitoyl transferase, B-galactocerebrosidase, glucocerebrosidase, acid/neutral/alkaline SMASE (sphingomyelinase), acid ceramidase, SM deacylase, GCdeacylase, pSAP, glucosylceramide synthase, ceramide synthase and sphingomyelin synthase), enzymes involved in synthesis, repair/maintenance and degradation of hyaluronic acid (e.g. hyaluron/hyaluronan synthases 1-3 and others), enzymes involved in cholesterol synthesis, maintenance/repair and degradation, enzymes involved in fatty acid, triacylglyceride, phospholipids, plasmalogen, sphingolipid, and eicosanoid (including the arachidonic acid cascade, prostaglandins and leukotrienes) synthesis, maintenance/repair and degradation, retinoic acid receptors, steroid receptors, hormone receptors (e.g. estrogen receptors), thyroid receptors, vitamin D receptors, peroxisomal proliferators-activated receptors (PPARs), farnesol-activated receptors (FXR), liver-activated receptor (LXR), matrix metalloproteinases (MMPs including collagenases (MMP-1), gelatinases (e.g., MMP-2 and MMP-9), elastases, stromelysins, serine proteases and membrane-type MMPSs), tissue inhibitors of metalloproteinases (TIMPs).

In yet other embodiments, the polynucleotide may encode (or be complementary to a nucleotide that encodes) a polypeptide or nucleic acid that encodes for at least a fragment of a serine proteinase inhibitors (e.g. skin-derived antileukoproteinase (SKALP), also known as elafin (preproelafin and proelafin included) and secretory leukocyte protease inhibitor (SLPI)), MSX (synonyms inclue CHOX-8; GHOX-8; HOMEOBOX PROTEIN MSX-2; HOX-8; HOX-8.1; HOX8; MSH HOMEO BOX HOMOLOG 2; MSX-1; MSX-2; MSX1; and QUOX-7), SMAD pathway, Smad3, c-ski, and extracellular signal-regulated kinase ½, cardiac repeat protein, estrogen-responsive B box protein (EBBP), IGF-binding proteins (IGFBPs), vitronectin, follistatin. RAC1, ADAMTS1 Proteinase, amphiphysin-1, transcription factor AP-1, cjun, cFos, beta-amyloid precursor protein (sAPP), Beta-catenin, CHL1, Corticotropin-releasing hormone (CRH), DJ-1, sterol-regulatory element binding proteins (e.g. SREBP-1 and SREBP-2), Phosphatidylinositol 3-Kinase (PI3K), stearoyl-CoA desaturase (SCD), fatty acid synthase, hydroxymethylglutaryl-CoA synthase (HMGCS), CCAAT/enhancer binding protein alpha (C/EBPalpha), C/EBPdelta, FAS, stearoyl coenzyme A desaturase-1 (SCD-1), enzymes involved in estrogen synthesis (e.g. aromatase), fibroblast growth factor homologous factor (FHF) polypeptides, decorin, lumican, fibromodulin (and other small leucine-rich repeat proteoglycans), versican, biglycan, aggrecan, brevican, galectins (e.g. galectin-3 and galectin-7), heparin sulfate, perlecan, syndecan-1, chondroiton sulfate, JUN-regulated factors (e.g. pleiotrophin (PTN) and stromal cell-derived factor 1 (SDF-1)), CXCR4 (SDF-1 receptor), anti-apoptotic proteins (e.g. Bcl2 and survivin), transcription factor nuclear factor (NF)-kappaB, nitric oxide synthase, enzymes involved in the synthesis and degradation of catecholamines, Mitogen-activated protein kinase (MAPK), transcription factor NF-E2-related factors (e.g. Nrf2 and Nrf3), p21-activated protein kinase 4 (PAK-4), enzymes involved in the synthesis of sphingosylphosphorylcholine (SPC), lipases, lipid-mobilizing peptides (e.g. beta-lipotropin and “lipolytic peptide A and peptide B”), amphiregulin. tyrosinase and other enzymes involved in melanin, eumelanin and pheomelanin synthesis and degradation, melanocortins (pituitary peptide hormones that include adrenocorticotropin (ACTH) and the alpha, beta and gamma melanocyte-stimulating hormones (MSH), prohormone proopiomelanocortin, a-MSH, ACTH, endothelin 1, camp, PKCb, bey1, bey2, bey3, human melanocortin-1 receptor (MC1R), OCA1 (tyrosinase, TYR), OCA2 (OCA2), OCA3 (tyrosinase-related protein 1, TYRP1), and OCA4 (membrane-associated transporter protein, MATP), c-Jun N-terminal kinase kinase kinases (JNKKK polypeptides e.g. MLK4, PAK-4, PAK5 and YSK2), Hifl-alpha regulated genes (e.g. BNIP3, hypoxia-induced gene 1, adenylate kinase 4, galactokinase, galectin-3, gelsolin, RhoA, Rho kinase, heterogeneous nuclear ribonucleoprotein H1 and splicing factor and REV3), alpha-SMA, S1-P, GRO-alpha/CXCR-1, erbB, HGF activator (HGFA), keratinocyte proline-rich protein (KPRP), neurotransmitters and neurotransmitter blockers (e.g. GABA (gamma-aminobutyric acid)), acetylcholine blockers, Waglerin 1 and Curare.

In yet other embodiments, the polynucleotide of the methods and/or compositions of the present invention may encode (or be complementary to a nucleotide that encodes) at least one of the following polypeptides (or fragments thereof): alpha 1-antitrypsin (an irreversible neutrophil elastase inhibitor that improves vein strength for varicose veins) that in certain embodiments, may be coupled with MMP-2 inhibition (MMP-2 is high in varicose vein tissues), Hormone-sensitive lipase (HSL), Protein Kinase A (activates HSL), Triglyceride lipase (ATGL), CGI-58 (a recently identified coactivator of ATGL that stimulates TG hydrolase activity in wild-type and HSL-deficient WAT but not in ATGL-deficient WAT suggesting that ATGL is the sole target for CGI-58 mediated activation of adipose lipolysis; together, ATGL and HSL are responsible for more than 95% of the TG hydrolase activity present in murine WAT). Additionally known lipases, atrial natriuretic peptides, trypsin, alpha-chymotrypsin, skin anti-microbial peptides to fight infection and acne (e.g. SLPI, lysozyme, and defensins), upstream transcription factor-1 (USF-1), DeltaNp63alpha, Sirt1, angiotensin (Ang) II type 1 (AT1) and type 2 (AT2) receptors, angiotensin II, SHP-1 (Src homology 2-containing protein-tyrosine phosphatase-1), and/or prostacyclin synthase (PGIS) may be encoded by, or targeted by antisense constructs of the present invention.

Additionally, the gene constructs of this present invention may comprise a polynucleotide (or fragment thereof) that encodes (or is complementary to a nucleotide that encodes) at least one of the following polypeptides (or fragments thereof) involved in cosmetic improvement or cosmetic maintenance: actin, procollagen or collagen fragments (e.g. KTTKS, polylysine peptides), tropoelastin or elastin fragments, laminin fragments, fibronectin fragments, Matrixyl™ (Pal-KTTKS), Matrixyl 3000™ (Pal-GHK and Pal-GQPR), RonaCare ASCIII™, CPC Peptide™, Collaxyl™, Peptide Vinci 01™, ALDENINE™, Ameliox™ (carnosine di-peptide), ANTARCTICINE™, BIOPEPTIDE CL™, BioPeptide-EL™, Kappa Elastin™, SYN®-COLL, thrombospondin TSP-1 fragment) MYOXINOL™, DERMAXYL™, copper peptides (GHK-Cu), CYTOKINOL® LS 9028, Peptamide 6™, RIGIN™, KOLLAREN®, (hepatocyte growth factor (HGF) active fragment), Eyeliss™, Haloxyl™, Dipeptide 2, Dermican (TSH fragment), and numerous soy-derived peptides and the like such as those listed in the CFTA's cosmetic ingredient INCI database. Additional topically applied peptides that can relax muscles include SYN®-AKE (Waglerin 1 mimic), VIALOX™ (Curare mimic), ARGIRELINE™ (Acetyl Hexapeptide-3), Leuphasyl™, and SNAP-8™ (same as SNAP-25). Additional, polypeptides having activity in skin are described in U.S. Pat. No. 6,586,185.

In yet other embodiments, the polynucleotide may encode for nucleic acid and/or polypeptide that acts to decrease, slow and/or delay senescence in cells having a cosmetic function such as peptides described in U.S. Pat. No. 6,953,664, so as to enhance and/or maintain a biochemical and/or physiological process that has a positive effect on cosmetic appearance.

In yet other embodiments, the polynucleotide may encode for an antibody to a protein in cells having a cosmetic function so as to enhance and/or maintain a biochemical and/or physiological process that has a positive effect on cosmetic appearance.

Combinations of Polypeptides

Also, the polynucleotide delivered by the constructs of the present invention may, in certain embodiments, comprise a polynucleotide that encodes for combinations of two or more polypeptides or nucleic acids. For example, combinations of certain growth factors may show a synergistic improvement in wound healing as compared to singly applied growth factors (see e.g., Lynch, 1999; J. Clin. Invest., 84:640-646; Jeschke et al., 2004, Gene Therapy 11:847-855; Sprugel et al., Wound Repair Regen., 2004, 12:67-79; Endocrine Reviews, 2002; 24:737-767; Nabarro, J. D., 1987, Clin. Endocrinol., 26:481-512; Ristow et al., 1988, J. Cell Physiol., 137:277-284; O'Keefe et al., 1988, J. Invest. Dermatol., 90:2-7; Cook et al., J. Cell Physiol., 146:277-189). In alternate embodiments, any one of the known isoforms of such proteins may be used. Also, the constructs may be administered such that the polypeptides are expressed in a 1:1 ratio. Or, other ratios (e.g., 2:1, 3:1, 4:1, 5:1, 10:1, 20:1, 50:1, 100:1, 500:1, or 1000:1 for each of the proteins may be used.

For example, in an embodiment, the combination may comprise a PGDF and an IGF. The PGDF may comprise any one of the known PDGF isoforms. Similarly, the IGF may comprise any one of the known isoforms. The constructs may be administered such that the polypeptides are expressed in a 1:1 ratio. Or, other ratios (e.g., 2:1, 3:1, 4:1, 5:1, 10:1, 20:1, 50:1, 100:1, 500:1, or 1000:1 for each of the proteins may be used. In one example embodiment, the constructs are administered to result in about 2:1 ratio (by weight) of PDGF-BB and IGF-1. Or other combinations of PDGF and/or IGF and/or other ratios may be used. Similarly, a combination of a PDGF and a TGF-alpha may be used. Or, combinations of HGF and IGF may be used. In yet other embodiments, a combination of PDGF and a basic FGF may be used, or a combination of HGF and basic FGF may be used. In yet another embodiment, a combination of GH and TGF-1 or TGF-II may be used.

In certain embodiments, the construct, or constructs may encode for more than two proteins. For example, in some embodiments, a combination of PDGF with several other growth factors may be used. In an embodiment, PDGF in combination with KGF, IGF and IGFBP may be used. Or, sub-combinations of these growth factors may be used.

Removal of Degraded Collagen and/or Other Debris

In yet other embodiments, the polynucleotide constructs of the present invention may encode for proteins that help to remove degraded biomolecules as a means to maintain and/or improve synthesis of intact biomolecules that are important to enhancing and/or maintaining a physiological process that has positive effect on cosmetic appearance. For example, in one embodiment, the construct may encode for MMP-2 and/or MMP-9 to remove collagen debris from MMP-1. The use of these constructs may then be followed by the use of constructs that encode for the formation of new collagen. Such constructs may, in certain embodiments encode for growth factors, collagen and/or TIMP-1.

Receptors

Additionally, polynucleotide delivered by the constructs of the present invention may, in certain embodiments, comprise a polynucleotide that encodes for a receptor or combination of receptors for the polypeptides, growth factors and other biomolecules (e.g., required co-factors or transcription factors) in cells having cosmetic function (e.g. KGF receptors, TGF receptors, FGF receptors such as FGFRiiib, etc.) to enhance and/or maintain a biochemical and/or physiological process that has a positive effect on cosmetic appearance.

Nature of Expression

The constructs of the present invention may be administered into one or more types of cells having a cosmetic function in the mammal, for example transfection of undifferentiated “stem” or “basal” skin cells, differentiating skin cells at various stages, and terminally differentiated skin cells. In an embodiment, the technique may provide for the stable transfer of the nucleic acid to the genome of the cells, so that the nucleic acid or polypeptide may be expressed long-term by the cells. Once incorporated into the genome of a stem cell, the polynucleotide that encodes at least one of a nucleic acid or a polypeptide involved in the maintaining the cells having cosmetic function to enhance and/or maintain a biochemical and/or physiological process that has a positive effect on cosmetic appearance may be heritable and expressible by its cell progeny such as keratinocyte or fibroblast cells derived from undifferentiated stem or basal cells. For example, an integrative vector may be used (e.g., retroviruses, lentivirus, AAV, “Sleeping Beauty” transposons, or bacterial phage integrases, such as ΦC31, and the like, such that the targeted cell is any cell having cosmetic function.

In other embodiments, the technique may provide for the transient transfer of the nucleic acid to the cells having cosmetic function, so that the nucleic acid or polypeptide may be expressed transiently by the cells. In alternate embodiments, such expression may be for seconds, minutes, hours, days, weeks, months or years.

In other embodiments, expression is transient due to the biology of the cell. In the case of skin cells, cell turnover may result in loss of the modified cells upon terminal differentiation such as differentiating keratinocyte cells that move upward toward the outer periphery of the epidermis to form squames which are eventually lost via desquamation. Or, the stem cells may be modified, but the nucleic acid or a polypeptide that enhances a biochemical and/or physiological process that has a positive effect on cosmetic appearance may not be expressed until some stage of the skin cell differentiation. Thus, although the genetic modification is maintained in the stem cell line, the expression of the nucleic acid or a polypeptide may depend on the developmental stage of the progeny cell, thereby allowing the modification to be transient based on differentiation stage. (i.e., either limited to the stem cell stage, or to a differentiated cell stage).

In certain embodiments, the expressed nucleic acid and/or polypeptide may exert paracrine and/or autocrine effects in cells having a cosmetic function between any combination of stem cells, differentiating cells and differentiated cells. For example, transfected dermal fibroblast stem cells may express and secrete growth factors (e.g. Keratinocyte Growth Factor (KGF)) that have an autocrine effect on differentiating keratinocytes in the epidermis. As another example, transfected differentiating keratinocytes in the epidermis may express and secrete growth factors (e.g. Transforming Growth Factor-alpha) that have an autocrine effect on other differentiating keratinocytes in the epidermis. Additionally, the expressed nucleic acid and/or polypeptide may be designed to change a normal paracrine function to an autocrine function. For example, dermal fibroblasts normally express and secrete Keratinocyte Growth Factor (KGF) which subsequently diffuses to the epidermal keratinocytes to exert a paracrine effect. However, epidermal keratinocytes can be transfected with KGF-encoding construct so that subsequent expression and secretion of KGF is changed to an autocrine function instead of KGF's normal paracrine function.

Sites of Administration

The constructs of the present invention may be administered into one or more sites of a subject to enhance and/or maintain a biochemical and/or physiological process that has a positive effect on cosmetic appearance. Sites of administration may include the forehead, scalp, hair follicles, upper eyelids, lower eyelids, eyebrows, eyelashes, infraorbital and periorbital areas (including typical “crows feet” areas), temples, nose, nose bridge, cheeks, tongue, nasolabial folds, lips and periobicular areas including “jowls” area, jaw line, ears, neck, breasts, under triceps, back of hands, back, abdomen, sides, buttocks, front and back of thighs, knees and other areas of tissue having a cosmetic function in the subject. In an embodiment, the constructs may be injected into the blood and expression of the polypeptide of interest targeted to a particular subset of cells having a cosmetic function as for example by the use of receptor-mediated delivery system or via tissue-specific regulatory sequences (e.g. promotors and/or signal sequences).

Celebrity Genes

In certain embodiments, the sequences of the constructs of the present invention can be derived from “celebrities” and public figures such as well-known actors, actresses, musicians, painters, authors, politicians, royalty, athletes, business leaders, ministers, activists, scientists, heroes, and the like, living or deceased from any available DNA source, or from such celebrities' familial blood relative line (e.g., from the daughter of a famous singer). In other embodiments, the construct sequences of the present invention can be derived from commonly known polymorphisms from the general mammalian population; from a mammal other than the mammal receiving the cosmetic modification (e.g. construct copies from a wife applied to a husband and vice versa); autologous from the same skin cell area or from a preferred skin cell area to the modification area; blood relative of the mammal being transfected (e.g., construct copies from a child applied to a parent): marital partner or companion of the mammal being transfected; other nationality or “race” (e.g. construct copies from French men used for transfection of American men); opposite gender (e.g. women may desire constructs from a famous male actor); or construct sequences from any non-mammalian sources (e.g algae, yeast, fungi, viruses, plants, fish and insects).

Molecular Constructs

In certain embodiments, the introduction and subsequent expression of the constructs of the present invention may be enhanced by molecular methods known in the art, including the use of gene construct alterations including modified internucleotides, targeted expression vectors (e.g. to keratinocyte and fibroblast stem cells), nuclear targeting, use of cell-specific, or developmentally regulated promoters and other elements (e.g., enhancer elements). The constructs of the present invention may comprise additional molecular elements to promote the expression and/or proper functioning of the polynucleotide or polypeptide involved in enhancing and/or maintaining cells having a cosmetic function.

Thus, in some embodiments, the constructs may comprise constitutive or inducible promoters operably linked to the nucleic acid that encodes for a polynucleotide or polypeptide involved in maintenance of cells having a cosmetic function so as to enhance and/or maintain a biochemical and/or physiological process that has a positive effect on cosmetic appearance.

In other embodiments, the constructs may comprise enhancer elements operably linked to the polynucleotide or polypeptide involved in maintenance in the cells having a cosmetic function so as to enhance and/or maintain a biochemical and/or physiological process that has a positive effect on cosmetic appearance.

Additionally or alternatively, the constructs may comprise cleavage site sequences, intron sequences, cap sites, or functional polyA elements operably linked to the polynucleotide or polypeptide involved in maintaining cells having a cosmetic function so as to enhance and/or maintain a biochemical and/or physiological process that has a positive effect on cosmetic appearance.

Methods of Administration

The constructs of the present invention may be administered to cells having cosmetic function using transfection methods known in the art. Numerous techniques are known in the art for the introduction of genes into cells and may be used in accordance with the present invention, provided that the necessary developmental and physiological functions of the recipient cells are not disrupted.

Methods for genetic transformation of cells having cosmetic function may include lipid-based delivery systems such as liposomes or biphasic vesicles. Also, emulsions used to permeate the cells having cosmetic function such as cationic nanoparticles, ethanol-in-fluorocarbon microemulsions, or water-in-oil and oil-in-water nanoemulsions may be used.

Or, concatemers may be used. Concatemers, constructed in vitro by treatment of mature DNA with T4-ligase also have an increased activity in transfection, as the transrerction does not have a requirement for more than one molecule per transfection event as is typically found for transfection smaller DNAs perhaps because the structure of the ends of the transfecting molecules play an important role intransfection.

In yet other embodiments, particle-mediated transfer may be used. In certain embodiments, electromagnetic radiation may be used to stimulate the ability of the constructs of the present invention to permeate cells having cosmetic function. Such methods may include the use of radio frequency to form microchannels, electroporation, iontophoresis, or the use of electroincorporation. Administration may also employ physical delivery as for example microinjection of naked DNA. The technique employed may allow for transient expression of the polynucleotide or polypeptide involved in skin maintenance or repair.

Or, techniques for long-lasting gene expression may be used. For example, molecular techniques such as infection with a viral or bacteriophage vector containing the nucleic acid sequences of interest may be employed. Also other techniques such as cell fusion, chromosome-mediated gene transfer, microcell-mediated gene transfer, spheroplast fusion, or transposons, are encompassed by the methods and compositions of the present invention. As is known in the art, chemical and physical (e.g., heat, pre-treatment microdermabrasion and occlusion) permeation enhancers may be used with any of the methods and compositions of the present invention.

Thus, embodiments of the present invention recognize the potential for the genetic modification of cells having cosmetic function as a means to improve the cosmetic appearance of the subject. Genetic modification of cells having cosmetic function may provide the means to either transiently or permanently modify such cells.

### Genes that May be Used to Modulate Skin Maintenance and/or Cosmetic Appearance

Undesirable changes in cosmetic appearance, such as sagging, thinning, or wrinkling of the skin is typically the result of the substantial and steady decline in the expression of skin proteins such as collagen, elastin, or other extracellular matrix proteins and proteoglycans. Therefore, replacing these proteins or preventing the decline of these proteins becomes a potentially effective strategy to maintain or improve the appearance and health of the skin. The examples described herein describe the design of four recombinant vector constructs that encode proteins designed to improve the stability of the extracellular matrix and support the improved appearance of the skin. These are collagen, elastin, EC-SOD, and TIMP-1. The role of these proteins in skin physiology is discussed in detail below.

Other proteins and/or peptides may also be provided using the methods and compositions of the present invention. For example, skin cells are known to express a variety of proteins and other factors that may be important to the repair and maintenance of the tissue. Thus, keratinocytes are known to produce interleukins (IL-1-alpha, IL-1-beta, IL-1ra, IL-3, IL-6, IL-8, IL-10, and IL-18), granulocyte colony-stimulating factor (G-CSF), monocyte colony-stimulating factor (M-CSF), and granulocyte/monocyte colony-stimulating factor (GM-CSF), transforming growth factors (TGF-alpha and TGF-beta), platelet-derived growth factor (PDGF), hepatocyte growth factor (HGF), vascular endothelial growth factor (VEGF), tumor necrosis factor (TNF-alpha), interferon (e.g., INF-alpha, beta, and gamma), insulin-like growth factor (IGF-1), and fibroblast growth factors (basic FGF, FGF-22). Also, fibroblasts are known to produce keratinocyte growth factors (KGF-1, KGF-2), TGF-beta-1, TGF-beta-2, and TGF-beta-3, connective tissue growth factor (CTGF), FGF-2 (basic), PDGF-A, IGF-1, VEGF, hepatocyte growth factor (HGF), IL-6, IL-8, TNF-alpha, GM-CSF ad G-CSF, FGF-22, and IGF-1. In another embodiment, the polynucleotide of interest is the HIF-1-alpha transcription factor. Also, it has been described that Egr-1 transcription factor polypeptides, or a biologically active fragment thereof, or nucleotides encoding such peptides, may be used to treat wounds (U.S. Pat. No. 6,689,758). Thus, any of these polypeptides, or functional derivatives thereof, may be used alone or in combination, in embodiments of the methods and/or compositions of the present invention.

Collagen

In an embodiment, the polypeptide involved in maintaining a cell having a cosmetic function so as to enhance and/or maintain a biochemical and/or physiological process that has a positive effect on cosmetic appearance may be a collagen polypeptide or a functional derivative thereof. Collagens are the main fibrous protein composing the extracellular matrices of the body's tissues. Type I collagen is the major extracellular matrix (ECM) component of human skin and is a triple-stranded helical structure composed of two α-1 and one α-2 chains (Myllyharju and Kivirikko, 2001, Ann. Med., 33:7-21). These procollagen chains are synthesized by fibroblasts in the skin and other tissues throughout the body. Once synthesized, the procollagen chains are cleaved to allow for aggregation and formation of larger collagen fibrils that make up a critical component of the ECM. Degradation or insufficient production of procollagen fibers leads to a loss of structural integrity of the ECM and results in physical changes in the overlying tissues such as the skin.

The loss or degradation of collagen is a normal part of aging, but it also is one of the major changes observed in photodamaged skin (Lavker, 1995, Cutaneous Aging: chronologic versus photoaging, In Photoaging, Ed., Gilchrest, B. A., Cambridge Mass., Blackwell Science, pp. 123-135; Fligiel et al., 2003, J. Invest. Dermatol., 120:842-848; Varani et al., 2001, Am. J. Pathol., 158:931-942). It has been reported that sustained down-regulation of collagen synthesis occurs in aged, sun-protected skin (Varani et al., 2000, J. Invest. Dermatol., 114:480-486) and in photodamaged skin (Griffiths et al., 1993, N. Engl. J. Med., 329:530-535). A number of mechanisms elucidating the causes for collagen loss during normal aging and as a result of photodamage have been proposed with increases in matrix metalloproteinases (MMPs) and decreases in skin fibroblast numbers (e.g., Fisher et al., 1996, Nature, 379:335-338; Fisher et al., 1997, N. Eng. J. Med., 337:1419-1428; Millis et al., 1992, Exp. Cell Res., 201:373-379; Burke et al., 1994, Exp. Gerontol., 29:37-53; Varani et al., 2000). Another mechanism at least partly responsible for the loss of collagen is the age-related decrease in collagen synthesis by skin fibroblasts. Thus, it has been demonstrated that compared to young skin, aged skin exhibited a lower amount of type I procollagen and that young fibroblasts synthesize more type I procollagen compared to young fibroblasts (Varani et al., 2006). In this study, a reduction in type I procollagen synthesis and content was associated with a more open space between collagen bundles and less contact between fibroblasts and collagen fibrils, suggesting less mechanical tension on fibroblasts. Earlier research demonstrated that when mechanical tension is reduced, collagen production may decline and production of MMPs may increase (Lambert et al., 1992, Lab. Invest. 66:444-451; Delvoye et al., 1991, J. Invest. Dermatol., 97:898-902). Thus, age-related reduction in fibroblast collagen synthesis may lead to reduced mechanical tension in the ECM, which further reduces collagen production (Varani et al., 2006). By replenishing the skin with copies of the type I procollagen gene, collagen production may be enhanced, providing the potential for improved mechanical tension in the ECM, which may subsequently enhance collagen production from existing fibroblasts.

Elastin

In an embodiment, the polypeptide involved in maintaining a cell having a cosmetic function so as to enhance and/or maintain a biochemical and/or physiological process that has a positive effect on cosmetic appearance may be an elastin polypeptide or a functional derivative thereof. Elastin is another critically important component of the ECM that provides elasticity and resilience to the skin (reviewed by Mithieux and Weiss, 2005, Adv. Protein Chem., 70:437-461). Elastin is formed through synthesis and lysyl oxidase—mediated crosslinking of its precursor molecule, tropoelastin. Elastin is a durable molecule that makes of about 90% of elastic fibers and constitutes 2-5% of the dry weight of skin. The formation of elastin and elastic fibers occurs primarily during fetal development and shortly after birth. With the exception of responses to injury, little elastin is synthesized during adulthood. While the main function of elastin is to provide elasticity to tissues, the elastin-laminin receptor (ELR) has been reported to be involved with skin fibroblast proliferation (Groult et al., 1991, Cell Biochem. Funct., 9:171-182).

Normal ageing has been associated with the degradation and loss of elastic fibers (Braverman et al., 1982, J. Invest. Dermatol., 78:434-443; Ashcroft et al., 1997a, J. Pathol., 183:80-89). It has been reported that in individuals between 30-50 years of age, the formation of cysts and lacunae were the main abnormality (Braverman et al., 1982). The formation of porous fibers was observed in individuals between 50-70 years old, but became more frequent in people over 70 years of age. Similarly, it has been shown that the sun-protected skin of older subjects had fragmented elastin fibers in the sub-epidermal area with additional fragmented elastin fibers below the sub-epidermal layer (Ashcroft et al., 1997a). More recent studies have confirmed a decrease in skin elastin content with age. It has been reported that the elastin staining intensity of sun-protected skin decreased from 49% in the 1^{st} decade of life to 30% in the 9^{th} decade of life (E1-Domyati et al., 2002, Exp. Dermatol., 11:398-405). Similarly, it has been reported that there is a 51% reduction in elastin content of buttock (sun-protected) skin between 20 and 80 years of age (Seite et al., 2006, J. Eur. Acad. Dermatol. Venereol., 20:980-987). Interestingly, these authors also reported a 44% reduction in elastin content of facial skin (severe sun exposure) between 50 and 70 years of age, though no change in elastin content was observed in forearm skin (moderate sun exposure). This loss of skin elastin with age is one of the primary factors associated with the loss of elasticity and resilience of the skin, leading to wrinkling and sagging. Providing exogenous copies of the elastin gene may enhance new elastin production and help offset the normal loss of elastin seen with aging. This in turn may help improve the appearance of the skin.

Tissue Inhibitor of Metalloproteinase-1 (TIMP-1)

In an embodiment, the polypeptide involved in maintaining a cell having a cosmetic function so as to enhance and/or maintain a biochemical and/or physiological process that has a positive effect on cosmetic appearance may be a Tissue Inhibitor of Metalloproteinase-1 (TIMP-1) or a functional derivative thereof. Matrix metalloproteinases (MMPs) are a group of enzymes (collagenases, gelatinases, and stromelysins) involved in the digestion and reorganization of the ECM (Woessner, 1994, Ann. NY Acad. Sci., 732:11-30) and are present in abundance in the skin (reviewed by Kahari and Saarialho-Kere, 1997, Exp. Dermatol., 6:199-214). MMP activity is regulated by tissue inhibitors of metalloproteinases (TIMPs) (Birkedal et al., 1993, Crit. Rev. Oral Biol. Med., 4:197-250). The overall turnover rate of the ECM is a function of the ratio of MMP to TIMP activity in the tissue.

During normal, chronological aging, MMP levels, particularly MMP-1, in the skin increase (Ashcroft et al., 1997b, Cell Tissue Res., 290:581-591; Varani et al., 2000). A similar increase in skin MMP levels is observed with photoaging (Fisher et al., 1996, 1997). Models of photoaging using ultraviolet (UV) light-induced aging of the skin demonstrate that MMP-1 may be the major enzyme responsible for collagen degradation in the skin (Brennnan et al., 2003, Photochem. Photobiol., 78:43-48). In contrast to increasing MMP concentrations, TIMP-1 levels in the skin may decrease with the normal aging (Ashcroft et al., 1997c, J. Pathol., 183:169-178). This combination of increasing levels of MMP and the decreasing levels of TIMP-1 with normal aging, can lead to an overall loss of the skin's collagen content. With the loss of collagen and subsequent loss of its supporting ECM, the skin shows the typical outward signs of aging including the appearance of lines and wrinkles and the loss of firmness. Recent studies have reported that various MMP inhibitors may reduce facial skin wrinkling and support collagen production (McDaniel et al., 2005, J. Cosmetic Derm., 4:167-173; Moon et al., 2006, Phytomedicine 13:707-711; Park et al., 2006, Photochem Photobiol., 82:574-578). As a result of these kinds of data, cosmetic ingredients such as EquiStat (Engelhard) and ECM-Protect (Atrium Biotechnologies) to inhibit skin MMP activity have been marketed successfully.

By treating with TIMP-1 cDNA plasmid constructs, embodiments of the present invention may enhance TIMP-1 synthesis. By increasing TIMP-1 production, the skin may be able to reduce the effects of endogenous MMPs on the skin. When both the collagen gene construct, discussed above, and the TIMP-1 gene construct are delivered simultaneously, embodiments of the methods and or compositions of the present invention can generate a two-pronged approach to improving the appearance of the skin. On one hand, the collagen gene construct should enhance collagen production to provide additional support to the skin's ECM, while administration of the TIMP-1 construct will protect both the new and existing collagen from degradation through MMP activity.

Extracellular Superoxide Dismutase (SOD-3)

In certain embodiments, the polypeptide involved in maintaining a cell having a cosmetic function so as to enhance and/or maintain a biochemical and/or physiological process that has a positive effect on cosmetic appearance may be an Extracellular Superoxide Dismutase (SOD-3) or a functional derivative thereof. The skin, as well as many other tissues, contains an endogenous antioxidant enzyme system that consists of three potent enzymes, superoxide dismuatase (SOD), glutathione peroxidase (GPX), and catalase (Steenvoorden et al., 1997, J. Photochem Photobiol B:Biology 41:1-10; Afaq and Mukhtar, 2001, J. Photochem Photobiol B: Biology, 63:61-69). SOD functions to catalyze the reduction of superoxide anion to the less toxic hydrogen peroxide, which is subsequently reduced to water and oxygen by GPX and catalase.

Changes in these enzymes have been reported during aging and photoaging. It has been demonstrated that SOD and catalase were higher in the epidermis than the dermis, while GPX was higher in the dermis than the epidermis in both young and old human skin (Rhie et al., 2001, J. Invest. Dermatol., 117:1212-1217). No significant changes were detected in SOD or GPX with natural or photoaging; however, catalase increased in the epidermis and decreased in the dermis of both natural and photoaged skin (Rhie et al., 2001). In contrast, others have demonstrated a substantial reduction in all three antioxidant enzymes in the stratum corneum and a reduction in CuZn-SOD in the epidermis in response to photoaging (Sander et al., 2002, J. Invest. Dermatol., 118:618-625). This loss of antioxidant enzyme activity coincided with increases in oxidized proteins in the upper dermis of photoaged skin.

Superoxide dismutase is present in the skin in three forms (Cu/Zn-SOD, Mn-SOD, and EC-SOD). Recent studies have reported that EC-SOD may be of particular importance for antioxidant and anti-aging benefits. Extracellular SOD mRNA is present in both the dermis and epidermis, though higher in the dermis, while EC-SOD protein was located in both the epidermis and dermis at high levels (Choung et al., 2004, Exp. Dermatol., 13:691-699). It was demonstrated that EC-SOD mRNA expression is enhanced by exposure to both UVA and UVB (Choung et al., 2004). Others have examined the expression of antioxidant genes in fibroblasts under normoxia and hyperoxia conditions (Serra et al., 2003, J. Biol. Chem., 278:6824-6830). In these studies, hyperoxia induced EC-SOD gene expression, particularly in fibroblasts with high antioxidant capacity. Furthermore, EC-SOD expression was inversely correlated with telomere shortening such that increased expression of EC-SOD was associated with slower telomere shortening, resulting in the extended replicative lifespan of fibroblasts (Serra et al., 2003). Extracellular SOD has also been shown to have direct protective effects on type I collagen. It has been demonstrated that EC-SOD binds directly to type I collagen via its C-terminal heparin-binding region (Petersen et al., 2004, J. Biol. Chem., 279:13705-13710). Additionally, these authors reported that the bound EC-SOD prevented the oxidative fragmentation of type I collagen. In addition to potential skin benefits, EC-SOD has been reported to have anti-inflammatory effects (Ha et al., 2006, Biochem. Biophys. Res. Comm., 348:450-458), to reduce chemically induced tumor formation (Kim et al., 2005, Oncol. Res., 15:333-341), to alleviate some symptoms of collagen-induced arthritis (Iyama et al., 2001, Arthritis Rheum., 44:2160-2167; Ross et al., 2004, Arthritis Rheum., 50:3702-3711)

Exogenously administered EC-SOD gene in a topical formulation is likely to have a similar effect to the over-expression of endogenous EC-SOD mRNA, i.e. enhance EC-SOD production, enhance cellular antioxidant capacity and enhance cellular lifespan. By enhancing the lifespan and antioxidant capacity of skin fibroblasts, these cells will be more functionally capable of producing and maintaining the ECM necessary for good skin health. Furthermore, the direct protective action of EC-SOD on collagen may help protect existing collagen from oxidative damage associated with aging and photoaging.

KGF

In an embodiment, the polypeptide involved in maintaining a cell having a cosmetic function so as to enhance and/or maintain a biochemical and/or physiological process that has a positive effect on cosmetic appearance may be a keratinocyte growth factor (KGF). KGF-1 (or FGF-7) is a heparin-binding growth factor that can promote the proliferation, migration, and morphogenesis of epithelial cells (see e.g., au dem Keller, Eur. J. Cell Biol., 2004, 11-12, 607-612). Also, KGF can stimulate production of collagen and/or elastin, both of which are important for maintaining healthy skin and other tissue that has a cosmetic function. The carboxy-terminal two thirds of this 26-28 kDa protein is 30-45% identical to eight other proteins in the FGF family (Rubin et al., Cell Biol. Int., 1995, 19, 399-411). KGF is produced by a variety of mesenchymal cells, but does not appear to be produced by epithelial cells. Epithelial cells express the high affinity KGF receptor, FGFR1-IIIb, which is the only FGF receptor bound by KGF (Grazu-Bilska et al., 2003; Werner et al., Cytokin Growth Factor Rev., 1998, 9, 153-65). KGF may work in combination with other growth factors. Thus, non-viral liposomal transfer of KGF cDNA into wounds resulted in increased expression of VEGF and IGF-1 (Grazu-Bilska et al., 2003). In an embodiment, KGF may be administered in a large enough amount to cause IGF-1, VEGF and PDGF expression.

KGF-2 (also known as either FGF-12), has 57% homology to KGF. Levels of KGF-2 increase wound healing similar to KGF-1. Also, a truncated form of KGF, KGF_{des1-23}, that has increased mitogenic activity has been described (U.S. Pat. No. 5,677,278). Repifermin is a truncated form of recombinant human KGF-2 that has been used in the healing of chronic venous stasis ulcers (Robson et al., Wound Repair Regen., 2001, 9, 347-52; Fricker, Mol. Med. Today, 1998, 4, 229). Also, recent evidence has demonstrated that dendritic gamma-delta epidermal T cells (DETCs) produce FGF-7 and FGF-12, which contribute to keratinocyte proliferation during would healing (Baum and Arpey, Dematol. Surg., 2005, 31, 674-686; Born et al., Nat. Med., 2002, 8, 560-1; Jaeson, Science, 2002, 296:747-9).

For example, polynucleotide constructs that encode KGF-1, KGF-2, and analogues thereof are described in U.S. Pat. Nos. 5,731,170, 5,677,278, 5,814,605, 6,228,839, and 6,916,786. Such sequences may be used in the polynucleotide constructs of the present invention as well as truncated versions such as Kepivance® (palifermin) which differs from endogenous human KGF in that the first 23 N-terminal amino acids have been deleted to improve stability.

IGF

In an embodiment, the polypeptide involved in maintaining a cell having a cosmetic function so as to enhance and/or maintain a biochemical and/or physiological process that has a positive effect on cosmetic appearance may be an insulin-like growth factor (IGF). Insulin-like growth factors are primarily produced in the liver, but may be produced by all cells via autocrine mechanisms. IGF-1 and IGF-2 may be involved in the regulation of tissue growth, development, and regeneration (see e.g., Grazul-Bilska et al., 2003). IGF-1 and PDGF may act together to accelerate healing of skin wounds, bone regeneration and periodontal wounds. For example, the delivery of very small amounts of IGF-1 cDNA via liposomes increased the rate of re-epithelialization in rats with burn wounds (Pierre et al, J. Burn Care Rehab., 1997, 18, 287-91; Jeschke et al., Gene Therapy, 1999, 6, 1015-1020). IGF-1 may interact with growth hormone in a synergistic manner (Meyer et al., J. Trauma, 1996, 41, 1008-12). IGF-1 may also, in certain embodiments, increase fat levels in cells. This may be beneficial for increasing the “plumpness” of the tissue, as for example, in facial skin or lips.

TGF-beta

In an embodiment, the polypeptide involved in maintaining a cell having a cosmetic function so as to enhance and/or maintain a biochemical and/or physiological process that has a positive effect on cosmetic appearance may be transforming growth factor-beta (TGF-beta). TGF-beta is synthesized by several cell types, including platelets, macrophages, lymphocytes, fibroblasts, bone cells, and keratinocytes. TGF-beta may stimulate production of fibronectin and collagen by fibroblasts and can increase the incorporation of these proteins into the extracellular matrix (Servoid, Clin. Pod. Med. Surg., 1991, 8, 937-53). Local applications of TGF-beta have beneficial effects on wound healing (Graham et al, J. Wound Care, 1998, 7, 536-40). Thus, TGF-beta may be involved in strengthening and modeling of skin tissue.

HIF-1

In an embodiment, the polypeptide involved in maintaining a cell having a cosmetic function so as to enhance and/or maintain a biochemical and/or physiological process that has a positive effect on cosmetic appearance may be a hypoxia inducible factor (HIF-1). HIF-1 is a DNA binding protein that can activate expression of genes that have an HIF-1 binding site. Examples of genes activated by HIF-1 include VEGF, erythropoietin, and glycolytic genes. HIF-1 is composed of two subunits, HIF-1-alpha and HIF-1-beta. In has been found that variants of either subunit may be used to inactivate HIF-1, by forming a nonfunctional HIF-1 dimer. Thus, in alternate embodiments, the nucleic acid or polypeptide involved in maintaining a cell having a cosmetic function is a polynucleotide that encodes for polypeptides that encode HIF-1-alpha, HIF-1-beta, variants thereof, or a nucleic acid comprising the HIF-1 binding site. Examples of such sequences are found in U.S. Pat. No. 5,882,914).

Other Polypeptides Involved in Skin Function

In an embodiment, the polypeptide involved in maintaining a cell having a cosmetic function so as to enhance and/or maintain a biochemical and/or physiological process that has a positive effect on cosmetic appearance may be Epidermal Growth Factor (EGF). EGF is a 6 kDa molecule that has about 30% amino acid homology with TGF-alpha. EGF is produced by platelets and is present in high concentrations during wound healing. EGF may increase the rate of epithelialization of wounds and may reduce scarring by preventing excess wound contraction. EGF may work in combination with KFG, PDGF and/or other growth factors to maintain and/or repair skin tissue.

In another embodiment, the polypeptide involved in maintaining a cell having a cosmetic function so as to enhance and/or maintain a biochemical and/or physiological process that has a positive effect on cosmetic appearance may be a member of the fibroblast growth factor family (FGF). The FGF family comprises several related polypeptides, including acidic FGF (aFGF or FGF-1), basic FGF (bFGF or FGF-2), several oncogenes (int-2, hst/K-FGF, FGF-5) and KGF (discussed above). The fibroblast growth factors believed to be most important in skin are FGF-1, FGF-2, and KGF. In other embodiments, FGF-10 and/or FGF-22 may be used (see e.g., au dem Keller, Eur. J. Cell Biol., 2004, 11-12, 607-612). FGFs may stimulate angiogenesis, as well as the proliferation and/or migration of many cells involved in wound healing, including capillary endothelial cells, vascular endothelial cells, fibroblasts, keratinocytes, epithelial cells, and specialized cell types such as chondrocytes and myoblasts. Also, FGF-2 may stimulate collagen synthesis, epithelialization and fibronectin and proteolycan synthesis (see e.g., Grazu-Bilska, 2003).

In another embodiment, the polypeptide involved in maintaining a cell having a cosmetic function so as to enhance and/or maintain a biochemical and/or physiological process that has a positive effect on cosmetic appearance is platelet-derived growth factor (e.g., PDGF AA, AB, BB, CC and others). PDGF is a potent mitogen for cells of mesenchymal origin and has been shown to promote wound healing. PDGF promotes collagenase production in fibroblasts, thereby facilitating migration of fibroblasts and remodeling of the wound matrix. For example, it has been found that ex vivo modification of keratinocytes to over-express platelet-derived growth factor (PDGF) can enhance wound healing (Eming et al., Hum. Gene Ther., 1998, 9, 529-539). Also

In yet another embodiment, the polypeptide involved in maintaining a cell having a cosmetic function so as to enhance and/or maintain a biochemical and/or physiological process that has a positive effect on cosmetic appearance may be vascular endothelial growth factor (VEGF). VEGF is highly conserved and shares structural homology with PDGF. VEGF is an endothelial cell-specific mitogen and chemoattractant with potent in vivo angiogenic activity (see e.g., Grazu-Bilska, 2003) which may be important for skin cell maintenance and repair.

In yet another embodiment, the polypeptide involved in maintaining a cell having a cosmetic function so as to enhance and/or maintain a biochemical and/or physiological process that has a positive effect on cosmetic appearance is hepatocyte growth factor (HGF) (see Ono et al., J Surg Res. 2004 July; 120(1):47-55) Hepatocyte growth factor (HGF) has a number of biological activities, e.g., mitogenic, motogenic, antiapoptotic, antifibrous, and morphogenic. It also has angiogenic and angioprotective activities for endothelial cells.

In yet other embodiments, the polypeptide involved in maintaining a cell having a cosmetic function so as to enhance and/or maintain a biochemical and/or physiological process that has a positive effect on cosmetic appearance is at least one of connective tissue growth factors (CTFG-1, CTFG-2), granulocyte-macrophage colony-stimulating factor (GMCSF) (see e.g. U.S. Pat. No. 6,689,351 describing treatment of wounds with GMCSF), macrophage colony stimulating factor (MCSF), growth hormone (GH). Also, the polypeptide involved in a cell having a cosmetic function so as to enhance and/or maintain a biochemical and/or physiological process that has a positive effect on cosmetic appearance may comprise an anti-angiogenic polypeptide, such as TSP-1 or TSP-2 (see e.g., U.S. Pat. No. 6,712,617). Other polypeptides and factors active in cells having a cosmetic function such as those described above, and in U.S. Pat. No. 6,586,185, incorporated by reference in its' entirety herein, may be used as well.

### Molecular Constructs and Methods of Making

In various embodiments, the gene of interest is produced by recombinant DNA technology. A recombinant DNA molecule comprising a polynucleotide encoding at least one of a nucleic acid or a polypeptide involved in maintaining a cell having a cosmetic function so as to enhance and/or maintain a biochemical and/or physiological process that has a positive effect on cosmetic appearance can be made and expressed by conventional gene expression technology using methods well-known in the art. In this way, the polypeptide or polynucleotide of interest having the appropriate flanking sequences may be produced in large quantities.

Each of the polynucleotides or polypeptides used in the methods and compositions described herein include biologically active derivatives, analogs and/or fragments that retain the biological activity of the full-length polynucleotide or polypeptide. Such derivative or analogs may include post-translationally modified polypeptides, for example, analogues generated by glycosylation, acetylation, or phosphorylation of the polypeptide. Or, polynucleotide analogues may be made by chemically synthesizing a nucleotide molecule with modified residues. Polypeptide analogs can be also made by conventional techniques of amino acid substitution, deletion, or addition, as for example, by site-directed mutagenesis. In one embodiment, a fragment of the gene of interest can be made by deleting either nucleotides and/or amino acid residues from the nucleic acid or a polypeptide involved in skin maintenance and/or treatment, respectively, as is known in the art.

The polynucleotide encoding at least one of a nucleic acid or a polypeptide involved in maintaining a cell having a cosmetic function so as to enhance and/or maintain a biochemical and/or physiological process that has a positive effect on cosmetic appearance can be propagated and/or expressed in a prokaryotic or eukaryotic expression system. In alternate embodiments, a bacterial, mammalian, yeast, or insect cell system may be used for propagation of the recombinant construct.

In one embodiment, a polynucleotide encoding at least one of a nucleic acid or a polypeptide involved in maintaining a cell having a cosmetic function so as to enhance and/or maintain a biochemical and/or physiological process that has a positive effect on cosmetic appearance can be expressed as a fusion protein by linking, in the correct frame and orientation, the coding sequence of the nucleic acid or a polypeptide involved in maintaining a cell having a cosmetic function to the coding sequence of another molecule. The fusion protein may be designed to increase the stability or the correct processing of the nucleic acid or a polypeptide involved in maintaining a cell having a cosmetic function. Or, the nucleic acid or a polypeptide involved in maintaining a cell having a cosmetic function so as to enhance and/or maintain a biochemical and/or physiological process that has a positive effect on cosmetic appearance can be conjugated to other molecules suitable for its intended use. For example, the nucleic acid or a polypeptide involved in maintaining a cell having a cosmetic function so as to enhance and/or maintain a biochemical and/or physiological process that has a positive effect on cosmetic appearance can be conjugated to a binding partner to a receptor that is recognized by the cell of interest.

The practice of the present invention will employ, unless otherwise indicated, conventional techniques of molecular biology, microbiology, recombinant DNA, and immunology, which are within the skill of the art. Such techniques are explained fully in the literature, including Sambrook, et al., Molecular Cloning: A Laboratory Manual 2nd ed. (Cold Spring Harbor Laboratory Press, 1989); DNA Cloning, Vol. I and II, D. N Glover ed. (IRL Press, 1985); B. Perbal, A Practical Guide To Molecular Cloning, Wiley (1984); Gene Transfer Vectors For Mammalian Cells, J. H. Miller and M. P. Calos eds. (Cold Spring Harbor Laboratory, 1987); Methods In Enzymology, Vol. 154 and 155, Wu and Grossman, eds., and Wu, ed., respectively (Academic Press, 1987).

Recombinant constructs comprising the nucleic acid or a polypeptide involved in maintaining a cell having a cosmetic function so as to enhance and/or maintain a biochemical and/or physiological process that has a positive effect on cosmetic appearance can be made by well-known recombinant techniques. In this regard, the sequence of interest may be operably linked to one or more regulatory sequences in a suitable vector in a proper reading frame and orientation. Thus, the sequence of interest may be inserted, for example, into a mammalian vector for expression in mammalian cells.

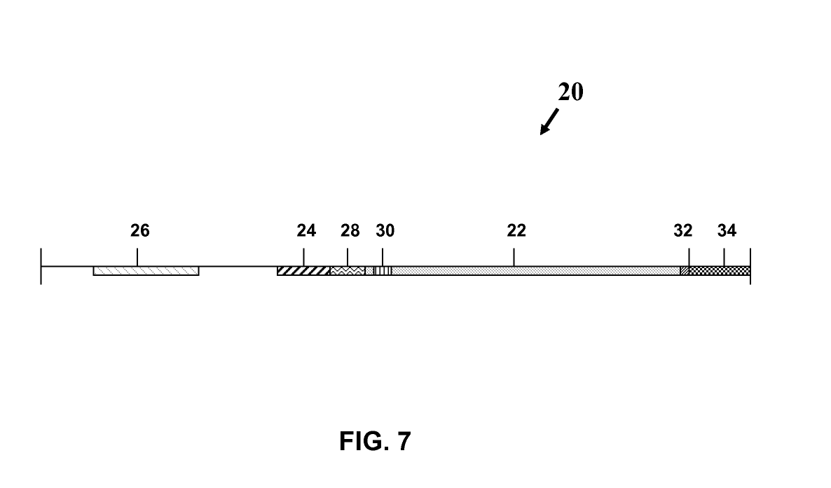

FIG. 7 shows an example recombinant construct 20 of the present invention. The recombinant construct may comprise a DNA molecule that encodes for a protein or polypeptide 22 involved in maintaining a cell having a cosmetic function so as to enhance and/or maintain a biochemical and/or physiological process that has a positive effect on cosmetic appearance. Or, the construct may comprise a polynucleotide (e.g., antisense RNA or siRNA) that may be used to regulate expression of polypeptide involved in maintaining a cell having a cosmetic function so as to enhance and/or maintain a biochemical and/or physiological process that has a positive effect on cosmetic appearance.

In an embodiment, the coding sequence of a polypeptide involved in maintaining a cell having a cosmetic function so as to enhance and/or maintain a biochemical and/or physiological process that has a positive effect on cosmetic appearance is derived from a complementary DNA (cDNA) made by reverse transcription of cellular RNA from a host cell known to express the gene of interest as described in the Examples herein, or using other methods known in the art.

As shown in FIGS. 7A and 7B, in certain embodiments, the recombinant construct may comprise a regulatory sequence such as a promoter 24 and/or an enhancer element 26. Thus, a regulatory sequence comprising a promoter 24 that is operable in the host cell of interest may then be linked to cDNA sequence using molecular techniques. Other regulatory sequences can also be used, such as one or more of an enhancer sequence 26, a ribosome binding site 28, an intron with functional splice donor and acceptance sites, a signal sequence for directing secretion of the recombinant polypeptide 30, a termination sequence 32, a polyadenylation signal and/or polyadenylation sequence 34, other transcription terminator sequences, and a sequence homologous to the host cell genome. Other sequences, such as an origin of replication, can be added to the vector as well to optimize expression of the desired product. Also, a selectable marker may be included in the vector for selection of the presence thereof in the transformed host cells.

The regulatory sequences may be derived from various sources. For example, one or more of them can be normally associated with the coding sequence, or may be derived from, or homologous with, regulator systems present in the host cell (e.g., a keratinocyte). Alternatively, the promoter may be derived from a gene that is turned on in response to compounds or conditions that promote degeneration of a cell having a cosmetic function, such as UV light or certain chemicals. The various components of the expression vector can be linked together directly or, via linkers that constitute sites of recognition by restriction enzymes as is known in the art.

Any promoter that would allow expression of the nucleic acid that encodes for a polynucleotide or a polypeptide involved in maintaining a cell having a cosmetic function so as to enhance and/or maintain a biochemical and/or physiological process that has a positive effect on cosmetic appearance can be used in the present invention. For example, mammalian promoter sequences that can be used herein are those from mammalian viruses that are highly expressed and that have a broad host range.

The promoter may be a promoter that is expressed constitutively in most mammalian cells such as the PKG promoter. Examples of suitable regulatable elements which make possible constitutive expression in eukaryotes are promoters which are recognized by the RNA polymerase III or viral promoters, CMV enhancer, CMV promoter, SV40 promoter or LTR promoters, e.g. from MMTV (mouse mammary tumor virus (e.g., Lee et al., 1981, Nature, 214, 228-232) and other viral promoter and activator sequences, derived from, for example, HBV, HCV, HSV, HPV, EBV, HTLV or HIV. Examples of regulatable elements which make possible regulatable expression in eukaryotes are the tetracycline operator in combination with a corresponding repressor (Gossen M., et al., 1994, Curr. Opin. Biotechnol., 5, 516-20).

In an embodiment, the expression of the gene of interest genes takes place under the control of tissue-specific promoters. In alternate embodiments, the tissue-specific promoters that may be used include skin-specific promoters such as, for example, the human K10 promoter (Bailleul et al., 1990, Cell, 62, 697-708; Sawamura et al., J. Invest. Dermatol., 1999, 112:828-830), the human K14 promoter (Vassar et al., 1989, Proc. Natl. Acad. Sci. USA, 86, 1563-67), or the bovine cytokeratin IV promoter.

Alternatively, the promoter may be a promoter that is turned on at a particular time in the cell cycle or developmental phase. For example, the constructs may comprise regulatable elements which make possible tissue-specific expression in eukaryotes, such as promoters or activator sequences from promoters or enhancers of those genes which code for proteins which are only expressed in certain cell types. Examples of regulatable elements which make possible cell cycle-specific expression in eukaryotes are promoters of the following genes: cdc25A, cdc25B, cdc25C, cyclin A, cyclin E, cdc2, E2F-1 to E2F-5, B-myb or DHFR (see e.g., U.S. Pat. No. 6,856,185; U.S. Pat. No. 6,903,078; and Zwicker J. and Muller R., 1997, Trends Genet., 13, 3-6). The use of cell cycle regulated promoters may be used where expression of the polypeptides or nucleic acids used according to the invention is to be restricted to proliferating cells. In an embodiment, an example of an regulatable element which allows for keratinocyte-specific expression in the skin is the FiRE-element (Jaakkola et al., 2000, Gen. Ther., 7, 1640-1647). The FiRE element is an AP-1-driven, FGF-inducible response element of the Syndecan-1 gene (Jaakkola et al., 1998, FASEB J., 12, 959-9). Also, examples of elements which make possible metabolically specific expression in eukaryotes are promoters that are regulated by hypoxia (e.g., HIF-1-alpha), by glucose deficiency, by phosphate concentration or by heat shock.

In another embodiment, an enhancer element can be combined with a promoter sequence. Such enhancers may not only amplify, but also can regulate expression of the gene of interest. In an embodiment, the enhancer may be derived from a sequence that is normally positioned adjacent to a gene that encodes for a polyppeptide (e.g., collagen) involved in the maintenance or function of cells involved in cosmetic appearance. Suitable enhancer elements for use in mammalian expression systems are, for example, those derived from viruses that have a broad host range, such as the SV40 early gene enhancer, the enhancer/promoters derived from the LTR of the Rous Sarcoma Virus, and from human cytomegalovirus. Additionally, other suitable enhancers include those that can be incorporated into promoter sequences that will become active only in the presence of an inducer, such as a hormone, a metal ion, or an enzyme substrate, as is known in the art.

In another embodiment of the present invention, a transcription termination sequence may be placed 3′ to the translation stop codon of the coding sequence for the gene of interest. Thus, the terminator sequence, together with the promoter, would flank the coding sequence.

The expression vector may also contain an origin of replication such that it can be maintained as a replicon, capable of autonomous replication and stable maintenance in a host. Such an origin of replication includes those that enable an expression vector to be reproduced at a high copy number in the presence of the appropriate proteins within the cell, for example, the 2μ and autonomously replicating sequences that are effective in yeast, and the origin of replication of the SV40 vital T-antigen, that is effective in COS-7 cells. Mammalian replication systems may include those derived from animal viruses that require trans-acting factors to replicate. For example, the replication system of papovaviruses, such as SV40, the polyomavirus that replicate to extremely high copy number in the presence of the appropriate vital T antigen may be used, or those derived from bovine papillomavirus and Epstein-Barr virus may be used.

In some cases, the expression vector can have more than one replication system, thus, allowing it to be maintained, for example, in mammalian cells for expression and in a procaryotic host for cloning and amplification (see e.g., U.S. Pat. No. 5,677,278).

In one embodiment, the expression vector can be made to integrate into the host cell genome as an integrating vector. The integrating vector herein may contains at least one polynucleotide sequence that is homologous to the host cell genome that allows the vector to integrate. For example, in one embodiment, bacteriophage or transposon insertion sequences may be used. Optimization of the techniques described herein may be performed as described in e.g., in Branski et al., 2006, Gene Therapy, 2006, 1-10; and Hengge, 2006, Gene Therapy, 13:155-1563.

In certain embodiments of the present invention, one or more selectable markers can be included in the expression vector to allow for the selection of the host cells that have been transformed. Selectable markers that can be expressed in a host cell include genes that can render the host cell resistant to drugs such as tunicamycin, G418, ampicillin, chloramphenicol, erythromycin, kanamycin (neomycin), and tetracycline. Selectable markers also include biosynthetic genes, such as those in the histidine, tryptophan, and leucine biosynthetic pathways, such as ade2, his4, leu2, trp1, or that provide the host cells with the ability to grow in the presence of toxic compounds, such as a metal, may be used.

Thus, in an embodiment, the present invention comprises a method of making a polynucleotide encoding at least one of a nucleic acid or a polypeptide involved in maintaining the cells having cosmetic function such that the nucleic acid or polypeptide can be expressed in cells having cosmetic function to enhance and/or maintain a biochemical and/or physiological process that has a positive effect on cosmetic appearance. The method may also comprise inserting the polynucleotide into a vector for expression of the recombinant construct. Thus, in certain embodiments, the present invention comprises an expression vector comprising polynucleotide encoding at least one of a nucleic acid or a polypeptide involved in maintaining the cells having cosmetic function. Also, in certain embodiments, the present invention comprises a host cell transfected with such a vector.

For example, the method may also comprise the step of incorporating the DNA construct into an expression vector. The method may further comprise the step of transfecting a cell with the expression vector of the present invention. Thus, in an embodiment, the present invention comprises a cell transfected with the expression vector of the invention. For example, plasmids may be constructed to express a polypeptide involved in maintaining the cells having cosmetic function. The expression cassette sequences may be inserted into an expression vector such as pcDNA3.1 or the expression vector (Invitrogen, CA) using standard recombinant techniques.

As is known in the art, such nucleic acid constructs may be modified by mutation, as for example, by PCR amplification of a nucleic acid template with primers comprising the mutation of interest. In this way, polypeptides comprising varying levels of biological activity may be designed. In alternate embodiments, the mutated sequences may be 70%, 75%, 80%, 85%, or 90% or more identical to the starting DNA. As such, variants may include nucleotide sequences that hybridize under stringent conditions (i.e., equivalent to about 20-27° C. below the melting temperature (TM) of the DNA duplex in 1 molar salt).

Also, the method may comprise transfecting the expression vector into a host cell. In certain embodiments, the polypeptides of the present invention may be expressed in mammalian expression systems, including systems in which the expression constructs are introduced into the mammalian cells using virus such as retrovirus or adenovirus. Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines may be selected through determining which cell lines have high expression levels of the polypeptide. Other cell lines that may be used are insect cell lines, such as Sf9 cells. Plant host cells may include, e.g., Nicotiana, Arabidopsis, duckweed, corn, wheat, potato, and the like. Bacterial host cells may include E. coli and Streptomyces species. Yeast host cells include Schizosaccharomyces pombe, Saccharomyces cerevisiae and Pichia pastoris.

When recombinant expression vectors encoding the genes of interest are introduced into mammalian host cells, the polypeptides of the present invention may be produced by culturing the host cells for a period of time sufficient to allow for expression of the polypeptide in the host cells or secretion of the polypeptide into the culture medium in which the host cells are grown. The expressed polypeptide may be recovered from the culture medium using standard protein purification methods.

Nucleic acid molecules encoding the polypeptide of the present invention and expression vectors comprising these nucleic acid molecules may be used for transfection of a suitable mammalian, plant, bacterial or yeast host cell. Transformation may be performed by any known method for introducing polynucleotides into a host cell. Methods for introduction of heterologous polynucleotides into mammalian cells are known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming plant cells are known in the art, including, e.g., Agrobacterium-mediated transformation, biolistic transformation, direct injection, electroporation and viral transformation. Methods of transforming bacterial and yeast cells are also known in the art.

An expression vector may also be delivered to an expression system using DNA biolistics, wherein the plasmid is precipitated onto microscopic particles, preferably gold, and the particles are propelled into a target cell or expression system. DNA biolistics techniques are well-known the art and devices, e.g., a “gene gun”, are commercially available for delivery of the microparticles in to a cell (e.g., Helios Gene Gun, Bio-Rad Labs., Hercules, Calif.) and into the skin (PMED Device, PowderMed Ltd., Oxford, UK).

Expression of the polypeptides of the present invention from production cell lines may be enhanced using a number of known techniques. For example, the glutamine synthetase gene expression system (the GS system) and the plasma-encoded neomycin resistance system are common approaches for enhancing expression under certain conditions.

When the polypeptides of the present invention are expressed by different cell lines, they may have different glycosylation patterns from each other. However, all polypeptides encoded by the nucleic acid molecules as described herein, or comprising the amino acid sequences as described herein, are part of the instant invention, regardless of the glycosylation of the polypeptide.

In one embodiment, the recombinant DNA construct may be transfected into Chinese Hamster Ovary cells and expression optimized. In alternate embodiments, the cells may produce 0.1 to 20 grams/liter, or 0.5 to 10 grams/liter, or about 1-2 grams/liter. For example, the recombinant vectors may be stably transfected into Chinese Hamster Ovary (CHO) cells, and cells expressing a polypeptide involved in the maintenance of cells having cosmetic function selected and cloned. In an embodiment, cells expressing the recombinant construct are selected for plasmid-encoded neomycin resistance by applying antibiotic G418. Individual clones may be selected and clones expressing high levels of recombinant protein as detected by Western Blot analysis of the cell supernatant may be expanded, and the gene product purified by affinity chromatography using Protein A columns.

### Transfer of a Recombinant Construct Encoding a Polynucleotide or Polypeptide Involved in Skin Maintenance and/or Treatment into Cells Having Cosmetic Function

A variety of methods may be used to transfer a polynucleotide encoding a polynucleotide or polypeptide involved in maintaining cells having cosmetic function so as to enhance and/or maintain a biochemical and/or physiological process that has a positive effect on cosmetic appearance. Thus, the formulations of the present invention may comprise specific components that facilitate transfer of proteins into cells.

To allow for the introduction of nucleic acids in a eukaryotic and/or prokaryotic cell by transfection, transformation or infection, the nucleic acid can be present as a plasmid, as part of a viral or non-viral vector. Suitable viral vectors may include baculoviruses, vaccinia viruses, lentiviruses (see e.g., Siprashvili and Khavari, Mol. Ther., 2004, 9, 93-100), adenoviruses, adeno-associated viruses and herpesviruses. Examples of vectors having gene therapy activity are virus vectors, for example adenovirus vectors or retroviral vectors (Lindemann et al., 1997, Mol. Med., 3, 466-76; Springer et al., 1998, Mol. Cell., 2, 549-58). Also, eukaryotic expression vectors are suitable in isolated form for gene therapy use, as naked DNA can penetrate into cells having cosmetic function on topical application (Hengge et al., 1996, J. Clin. Invest., 97, 2911-6; Yu et al., 1999, J. Invest. Dermatol., 112, 370-5). Another form of gene therapy vectors can be obtained by applying the above described nucleic acid to gold particles and shooting these into tissue, preferably into the skin, or cells with the aid of the so-called gene gun (Wang et al., 1999, J. Invest. Dermatol., 112, 775-81, Tuting et al., 1998, J. Invest. Dermatol., 111, 183-8). Several methods of gene transfection that may be used with the methods and compositions of the present invention are described in more detail below.

Naked DNA

In an embodiment, the recombinant polynucleotide encoding at least one of a nucleic acid or a polypeptide involved in maintaining a cell having a cosmetic function so as to enhance and/or maintain a biochemical and/or physiological process that has a positive effect on cosmetic appearance is directly injected, as “naked DNA,” into the cells having cosmetic function. Also, oligonucleotides (e.g., encoding RNAi) may be efficiently transferred to all cell layers in the epidermis after topical application.

In certain embodiments, the DNA may be administered in water or a buffer. Or, liposomes as described in more detail herein may be used. Also, in certain embodiments, the skin may be abraded, either by removing a portion of the hair (e.g., stripping), or by brushing, or by other methods known in the art (Choi et al., Skin Pharmacol. Appl Skin Physiol., 2003, 16:271-282; Choi et al., Current Drug Delivery, 2006, 3:37-45).

In other embodiments, gene delivery systems comprising peptides bound to DNA may be used (see e.g., Jensen, Expert Opin. Biol. Ther., 2004, 4, 1-6). In yet another embodiment, the vector may be introduced as “naked” expression vectors into a biocompatible matrix, for example a collagen matrix (Goldstein and Banadio, U.S. Pat. No. 5,962,427).

Several studies have reported that naked plasmid DNA can be successfully delivered across the skin (Fan et al., 1999, Nature Biotech., 17:870-872; Yu et al., 1999, J. Invest. Dermatol., 112:370-375; Kang et al., 2004, J. Gene Med., 6:1238-1246). For example, it was reported that topical application of DNA in a phosphate buffered saline (PBS) solution generated antibodies against bacterial β-galactosidase, indicating the transdermal delivery of the naked plasmid DNA (Fan et al. (1999). By comparing this delivery in mice with and without normal hair follicles, Fan et al. (1999) suggested that delivery was via the hair follicles. However, others reported that delivery of naked plasmid DNA resulted in reporter gene expression in both the hair follicles and the superficial keratinocytes of the superficial epidermis (Yu et al., 1999). It has also been demonstrated that topical application of 300 μg of plasmid DNA results in the delivery of approximately 1,000 ng plasmid DNA/g of tissue after 2 hours and approximately 100 ng plasmid DNA/g of tissue after 24 hours and that mRNA was expressed as early as day 1 after topical application (Kang et al., 2004).

Early research suggests that plasmid DNA may penetrate the stratum corneum through a system of lacunae that transiently form into pore pathways under appropriate conditions (Menon and Elias, 1997, Skin Pharmacol., 10:235-246). It has also been demonstrated that pathways formed of aqueous regions surrounded by polar lipids may facilitate the intercellular transfer of polar materials (Sznitowska et al., 1998, J. Pharm. Sci., 87:1109-1114). Also, uptake of plasmid DNA intracellularly by keratinocytes has recently been shown to be primarily by macropinocytosies and may involve the DNA binding proteins ezrin and moesin (Basner-Tschakajan et al., 2004, Gene Therapy 11:765-774).

The safety and distribution of naked plasmid DNA has been extensively reviewed (e.g., Hengge U R, Vol-Platzer B (eds), The Skin and Gene Therapy. Springer-Verlag: Berlin, 2001, pp. 67-80). Unlike viral vector deliver of DNA, naked plasmid cDNA does not elicit adverse immune responses, does not undergo insertional mutagenesis, and maintains promoter function. A potential concern has been the migration of plasmid DNA from the target site to other tissues and organs, however, it has been reported that integration of plasmid DNA into host DNA was not detected at any time point and that expression of the plasmid cDNA was transient such that 11 days after treatment, expressed RNA was only detectable in the treated skin and not in any other tissues (Hengge et al., 1995, Nat. Genet., 10:161-166). It is hypothesized that this loss of plasmid DNA is due to degradation via tissue nucleases and epidermal regeneration. Thus, gene therapy for the skin is considered safe due to the transient expression of the plasmid DNA and the rapid degradation of the plasmid DNA.

Liposome and Nanosome Mediated Transfer

For example, in alternate embodiments, liposomes and/or nanosomes may be used to facilitate transfer of a polynucleotide encoding a polypeptide involved in maintaining cells having cosmetic function so as to enhance and/or maintain a biochemical and/or physiological process that has a positive effect on cosmetic appearance. Liposomes are artificially-made small vesicles with a lipid bilayer membrane comprised of phospholipids (Jeschke, M. G. et al., Gene Ther., 12, 1718-24 (2005); U.S. Pat. No. 6,576,618). Nanosomes are very small liposomes but may be made in essentially the same manner as liposomes.

Nucleic acids, proteins, and other biological materials can be enclosed in liposomes and/or nanosomes for delivery to mammalian cells through fusion with the cell's plasma membrane. Liposomes and/or nanosomes may be an attractive delivery system because they are non-viral, stable and can interact with the cell membrane. Administration of liposomes and/or nanosomes directly to the skin can allow for the molecules enclosed within the bilayer to be delivered into cells having cosmetic function via fusion with cell membranes. The mode of transfer may be through endocytosis or via a follicular pathway. For example, liposomes and/or nanosomes can be subcutaneously injected to transfect cells in the dermis, leading to localized protein expression in the skin. Pre-treatment of the skin with empty liposomes and/or nanaosomes followed by naked DNA may also be employed.

Liposomes and/or nanosomes can be comprised of cationic, anionic, or neutral lipids, and mixtures thereof (Luo, D. & Saltzman, W. M., Nat. Biotech., 18, 33-37 (1999)). For DNA transfer, the lipids can also be modified chemically to incorporate chemical groups to facilitate DNA condensation or release. Cationic lipids, such as quaternary ammonium detergents, cationic derivatives of cholesterol and diacylglycerol, and lipid derivatives of polyamines, may be favored for cell transfection because they decrease the net negative charge of the DNA and facilitate its interaction with cell membranes (Nishikawa, M. & Huang, L., 2001, Hum. Gene Ther., 12, 861-70; Badea J. Biol. Gene Medi. 2005, 7:1200-1214). Other liposomes tha may be used include 1,2-dioleoyl-3-trimethylammonium-propane (DOTAP)/dioleylphosphatidylethanolamines (DOPE)/DNA lipoplexes (see e.g., Paasonen et al., 2005, Int. J. of Pharmaceutics, 307:188-193).

Neutral lipids, such as dioleoylphosphitylethanolamine (DOPE), glycerol dilaurate, polyoxyethylene-10-stearyl ether (POE-10), and cholesterol, may be added as ‘helper lipids’ in cationic-lipid DNA complexes to facilitate the release of the DNA from the endosome after endocytic uptake of the complex. Auxiliaries that increase DNA transfer, such as polymers or proteins that are bound to the DNA or synthetic peptide-DNA molecules that make it possible to transport DNA into the nucleus of the cell more efficiently can also be used (see e.g., Niidome, T. & Huang, L., Gene Ther., 9, 1647-52 (2002)). Thus, cationic polymers, such as polylysine or protamine, can be used in lipid-DNA complexes as they cause tight condensation of DNA, which prevents complex aggregation and nuclease degradation. For example, mixing 1,2-dioleoyl-3-(trimethylammonium)propane) (DOTAP) liposomes with protamine sulfate prior to mixing with plasmid DNA produced small 135 nm particles that were stable and resulted in a high level of gene expression in a variety of tissues (e.g., lung., liver, heart) (Li, S. et al., Gene Ther., 5, 930-37 (1998)). Inclusion of cholesterol as a helper lipid may increase the transfection efficiency of liposome-peptide-DNA complexes. Also, luciferase or β-galactosidase gene DNA may be precompacted with short peptides derived from human histone or protamine before addition of a cationic lipid (Lipofectamine RPR 115335 or RPR 120535) or polymer (polyethylenimine) to achieve enhanced transfection efficiency, even in the presence of serum (see e.g., Schwartz, B. et al., Gene Ther., 6, 282-92 (1999)).

As is known in the art, liposomes may be made by heating lipids to form a lipid phase (Wu, H. et al., Int. J. Pharmaceut., 221, 23-24 (2001)). An aqueous phase containing water, salts or buffer may then be mixed with the lipid phase by passing the mixture back and forth between syringes under cooling conditions, followed by sonication until a final liposome size of 100 to 140 nm is reached. The DNA or protein to be included in the liposome is then added (as a solution) by inversion mixing. This liposome preparation can then be applied directly to or injected into the skin. The choice of lipids used, their ratio, the concentration of DNA used in creating the liposomes and the amount of liposomes added to the skin will generally require empirical determination for optimization. Auxiliaries to facilitate DNA transfer, such as peptides, can be mixed with the DNA prior to adding to the liposome mixture but the DNA-auxiliary must maintain sufficiently high aqueous solubility to be properly encapsulated within the external lipid phase of the liposome.

Alternatively, small unilamellar vesicles can be prepared by ultrasonic treatment of a liposome suspension comprised of cationic lipids, such as Cytofectin GS 2888, mixed with 1,2-dioleyloxypropyl-3-trimethylammonium bromide (DOTMA) or dioleoylphosphati-dylethanolamine bromide (DPOE). After inversion mixing, the DNA or protein may be bound ionically to the surface of the liposomes, in a ratio that maintains a positive net charge on the complex while having DNA complexed to 100% of the liposomes. Also, dimerizable cationic thiol detergents may be used to prepare liposomes for delivery of DNA (see e.g., Dauty, E. et al., J. Am. Chem. Soc., 123, 9227-34 (2001)). Upon oxidation, the thiol groups in the lipid can convert to disulfides and cause the DNA-lipid complex to form a stable nanometric particle that can bind electrostatically to cell surface anionic heparin sulfate proteoglycans for cellular uptake. The small size of the nanoparticle, and its lipid bilayer, can facilitate transfer of the DNA into cells having cosmetic function. Once inside the cell, the reductive environment provided by intracellular glutathione reduces the disulfides back to thiols and releases the DNA.

Water-in-Oil and Oil-in-Water Nanoemulsion-Mediated Transfer

In another embodiment, a water-in-oil and/or oil in water nanoemulsion may be used to deliver a polynucleotide or polypeptide involved in maintaining a cell having a cosmetic function so as to enhance and/or maintain a biochemical and/or physiological process that has a positive effect on cosmetic appearance. Water-in-oil and/or oil-in-water nanoemulsions are thermodynamically stable liquid isotropic dispersions, composed of water, oil and surfactants, and may comprise a technique for transferring DNA or proteins (or other biomolecules) into mammalian cells (Wu, H. et al., Int. J. Pharmaceut., 221, 23-24 (2001); see also, U.S. Pat. Nos. 5,753,241, 6,274,150, 6,335,022, 6,464,990, 6,541,018, and 6,689,371).

Components that are considered biologically safe, such as polyoxyethylene 20 sorbitan monooleate (TWEEN® 80), sorbitan monooleate (Span® 80) and olive oil, can be used to minimize risk of irritation to human subjects. At defined stoichiometric ratios of the components, and with warming and gentle mixing, spontaneous formation of the nanoemulsion may occur upon. Thus, there may not be a need for high shear forces, thereby eliminating a significant cause of physical damage to the biomolecule to be delivered. This technique can be readily scalable and can encapsulate significant amounts of aqueous phase. Nanoemulsions can be applied directly to the skin to achieve transfer of biomolecules into cells having cosmetic function. For example, this technique has been used to successfully transfect excised murine skin with either chloramphenicol acetyltransferase or human interferon-α2 cDNA such that DNA deposition was primarily into follicular keratinocytes (Wu et al., 2001). Transgene expression after a single application was found to be highest at 24 hours, although expression was significantly higher with multiple daily doses. Microemulsions including ethanol-in-fluorocarbon can also be used in a similar fashion.

Particle-Mediated Transfer

In yet another embodiment, the method of transfer may comprise particle-mediated transfer (see e.g., Nishikawa, M. & Huang, L., Hum. Gene Ther., 12, 861-70 (2001); Luo, D. & Saltzman, W. M., Nat. Biotech., 18, 33-37 (1999)). For example, DNA-coated microparticles, composed of metals such as gold or tungsten and 1-5 μm in size, may be accelerated to high velocity using a so-called gene gun to penetrate cell membranes (Williams, R. S. et al., Proc. Nat. Acad. Sci. U.S.A., 88, 2726-30 (1991)). Microparticles may be coated with DNA by mixing the particles in an aqueous slurry sequentially with the DNA solution, calcium chloride and free-base spermidine. The particles are then washed with an ethanol solution and spread onto the gene gun device applicator. Once the particles are dry and the ethanol has evaporated, the device can be fired to introduce the particles into the cell having a cosmetic function so as to enhance and/or maintain a biochemical and/or physiological process that has a positive effect on cosmetic appearance. This technique can introduce a biomolecule into many cells simultaneously, and may transfer the biomolecule through the cell membrane and into the cytoplasm or even the nucleus, thereby bypassing the endosomal compartment and potential enzymatic degradation. Also, in an embodiment, this technique may only achieve shallow penetration of the cell layer, which can be effective for limited local expression of the delivered DNA to the epidermis. Little cell damage or inflammation is caused by particle bombardment. For example, Oshikawa et al. used a helium-pulse PowderJect XR gene delivery device to project gold particles coated with interleukin 12 gene DNA into murine skin either overlying or distal to tumor locations (Oshikawa, K. et al., Hum. Gene Ther., 12, 149-60 (2001)). It was found that transfection of skin cells overlying the tumor location was most effective at suppressing tumor growth, but that treatment had an antimetastatic effect irrespective of transfection site (Oshikawa et al., 2001). Similarly, a similar protocol was used to successfully transfect murine skin with DNA encoding the luciferase gene under the control of the human β-actin promoter such that 10-20% of the cells in the bombarded area expressed the transferred gene (Williams et al., 1991).

Voltage-Driven Transfer

Yet other methods of transfer such as electroporation, iontophoresis, and electroincorporation may be used (see e.g., Banga, A. K. et al., Int. J. Pharmaceut., 179, 1-19 (1999); Banga A. J. & Prausnitz, M. R., TIBTech, 16, 408, 12 (1998); Andre, F. & Mir, L. M., Gene Ther., 11, S33-42 (2004)). In these methods, a current is used to facilitate the transfer of DNA into cells after the DNA has been injected into or applied to the skin, preferably in a high ionic strength medium. Skin is an optimal tissue for the use of these techniques due to ease of administration. Use of these techniques may entail optimization of the dose of DNA, electrode shape (e.g. needle, caliper) and number, and the electrical field strength and duration. Decreased permeation of cells after treatment suggests that any damage caused by these techniques is reversible. Additionally, these techniques can be combined to achieve a high degree of DNA transfer with less irritation as a result of exposure to current.

Electroporation:

Electroporation involves the use of very short (μs-ms) high voltage (typically>100 V) electrical pulses to transiently permeabilize cell membranes, permitting cellular uptake of macromolecules (see e.g., Nishikawa, M. & Huang, L., Hum. Gene Ther., 12, 861-70 (2001)). The current can open up pores in cell membranes through which DNA or proteins can pass down a concentration gradient into the interior of the cell thereby enhancing and/or maintaining uptake of the biomolecule after injection/application. This technique has been used to transfect skin in vivo by intradermally injecting plasmids encoding reporter genes into porcine or murine skin and then administering electrical pulses using PulseAgile Electroporation equipment and software (CytoPulse Sciences, Hanover, Md.) (Drabick, J. J. et al., Molec. Ther., 3, 249-55 (2000)). Transfected cells were primarily located in the dermis and included adipocytes, fibroblasts and endothelial cells, amongst others.

Iontophoresis:

Iontophoresis involves the use of low voltage (typically 10 V or less) electrical pulses administered in high frequency pulses or as a continuous constant current (typically 0.5 mA/cm³ or less) to push a charged molecule into skin (Nishikawa, M. & Huang, L., Hum. Gene Ther., 12, 861-70 (2001)). An electrode of the same polarity as the charge on the molecule may be used to drive the charged molecule into the skin. For example, a negatively charged anode can be used to transfer negatively charged DNA molecules. Electrodes are usually very small and may be incorporated into a matrix that is applied to the skin after application of the DNA solution (Mikszta, J. A. et al., Nat. Med., 8, 415-19 (2002)). The technique creates new pores in the skin, similar to electroporation, in addition to using preexisting pathways, such as sweat glands, to effectuate transfer. In contrast to eletroporation, this technique is commonly used with humans. Delivery of the DNA is proportional to the current and the size of the electrode matrix applied to the skin. Thus, dosage is readily adjustable and can be optimized according to patient needs. Also, because iontophoresis is driven transfer, it may be less dependant on other biological variables, such as membrane penetration or intracellular components, than are other drug delivery systems (Banga, A. K. et al., Int. J. Pharmaceut., 179, 1-19 (1999)). In an embodiment, micorenhancer arrays may be used to transfect the cells having cosmetic function of BALB/c mice with naked luciferase gene DNA with significant (e.g., up to 2,800-fold enhanced) transgene gene activity compared to topical application controls Mikszta, J. A. et al., 2002).

Electroincorporation:

Electroincorporation is a modification of iontophoresis and electrophoresis where molecules encapsulated in vesicles or particles, such as microspheres or gold particles, are delivered into the skin by applying a pulse which causes a breakdown of the upper layer of the skin. The electrodes may be placed directly onto the location of interest and the particles applied directly to the skin. Dielectrophoresis and/or pressure is thought to drive the particles into the skin after breakdown of the skin's top layer (Zhang et al., Bioelectrochem. Bioenerg., 42, 283-92 (1997)). Also, pressure-mediated electroincorporation may be used.

Radio Frequency Ablation-Mediated Transfer

Also, in an embodiment, the method of DNA and/or protein transfer may comprise radio frequency ablation mediated-transfer. Thus, microchannels, or transient microconduits, can be created in the cell having a cosmetic function by radio frequency ablation (Birchall, J. et al., Int. J. Pharmaceut., 312, 15-23 (2006)). These channels may be large enough and have sufficient morphology and depth to permit delivery of 100 nm nanoparticles. In yet another embodiment, the method of DNA or protein transfer may comprise ultrasound-mediated transfer, or phonophoresis. A device that has been used to achieve DNA transfer through radio frequency ablation creation of microchannels is ViaDerm™. The device has an electronic controller unit and a disposable array of stainless steel electrodes (100 or 50 μm in length) at a density of 100 electrodes/cm² in a total area of 1.4 cm². Microchannels may bee created by applying a voltage of 290 or 330 V and an RF frequency of 100 kHz for 1-5 bursts lasting 700 μs. The skin can be treated prior to and after the application of DNA to the skin to increase transfer. For example, the ViaDerm™ device was used to transfect excised human skin with plasmid DNA containing the beta-galactosidase or the green fluorescent protein reporter genes (Birchall et al., 2006).

Ultrasound

Ultrasound can also increase the permeability of cell membranes to macromolecules such as DNA (see e.g., Newman, C. M., et al., Echocardiography, 18, 339-47 (2001); Niidome, T. & Huang, L., Gene Ther., 9, 1647-52 (2002)). For example, after injection of DNA into the skin, an irradiating ultrasonic wave may be used to facilitate transfer of the DNA into cells. This technique is commonly used with humans, usually in musculature, and is both flexible and safe. Ultrasound has also been combined with microbubbles, or ultrasound contrast agents, such as perfluoropropane-filled albumin microbubbles, to lower the threshold for cavitation of cells by ultrasound energy (Teupe, C. et al., Circulation, 105, 1104-09 (2002)). For example, ultrasound-mediated destruction of plasmid-loaded albumin microbubbles was used to transfect porcine coronary arteries with DNA encoding an activated form of endothelial nitric oxide synthase (eNOS) with significant protein expression and enhanced nitrous oxide-mediated relaxation of bradykinin-stimulated arteries (Teupe et al., 2002).

### Tissue-Specific, Self-Replicating and Integrating Plasmid Expression Systems to Facilitate Long-Lasting Gene Expression

In an embodiment, the method of DNA transfer may comprise the use of self-replicating and integrating plasmid expression systems (reviewed in Newman, C. M., et al., Echocardiography, 18, 339-47 (2001); Niidome, T. & Huang, L., Gene Ther., 9, 1647-52 (2002)). Also, in certain embodiments, the integration may be tissue or cell-type specific. Thus, at least some of the techniques described herein may be transient and may not be biologically targeted to specific cell types.

In an embodiment, tissue specific delivery can be achieved by incorporating protein or peptide ligands into DNA complexes to facilitate receptor-mediated targeting of cells that express certain receptors on their surface. Additionally or alternatively, DNA elements can be incorporated into the DNA transferred to cells such that the encoded gene can only be expressed in cells containing the corresponding protein factor. For example, a tissue-specific transcription factor binding site or promoter can be incorporated into the DNA molecule to be transferred. Such a technique was used to successfully transfected murine liver in vivo with plasmid DNA containing a human factor IX minigene sequence, including a portion of the first intron and 3′-untranslated region, under the control of the hepatic apolipoprotein E locus control region and α1-antitrypsin promoter and with the bovine growth hormone polyadenylation signal (Miao, C. H. et al., Mol. Ther., 1, 522-32 (2000)). Including these genetic elements in addition to the gene sequence itself resulted in increased gene expression in the therapeutic range that was sustained for at least ten months.

In other embodiments, long-lasting expression of transferred genes can be achieved by transfer of self-replicating DNA molecules into target cells. For example, Epstein Barr virus provides a system by which DNA plasmids can be maintained episomally and yet be heritable passed down through generations of cells (Shirakata M. & Hirai, K., J. Biochem., 123, 175-81 (1998)). This system may require that the transferred DNA contains the Epstein Barr Nuclear Antigen 1 (EBNA1) coding sequence and the oriP DNA element such that after transfer into cells, expression of the EBNA1 protein can result in replication of the transferred DNA in conjunction with the genomic DNA. For long-lasting expression of the desired protein in the skin, the transferred DNA may need to be introduced into basal cells. For example, this system as been used for suicide gene therapy in vitro and in vivo using plasmids encoding the EBV elements and the herpes simplex virus type 1 thymidine kinase (HSV-1 tk) gene, to increase cell sensitivity to the chemotherapeutic drug ganciclovir (Maruyama-Tabata, H. et al., Gene Ther., 7, 53-60 (2000)). Also, long term expression of β₂-adrenergic receptor was achieved by injecting plasmid DNA encoding the gene and carrying the EBV elements into hamster ventricle muscle (Tomiyasu, K. et al. Gene Ther., 7, 2087-93 (2000)).

If the transferred plasmid DNA is integrated into the chromosomal DNA of the basal cells of the skin or other cell having a cosmetic function, it can be heritably transmitted to daughter cells during cell division and, thus, provide a continual source for protein expression. Integration of the transferred DNA into the chromosomal DNA, may employ the use of an enzyme that cleaves the chromosomal DNA for insertion of the transferred DNA. One method of effecting DNA incorporation into the chromosome of a cell having a cosmetic function may be the use of transposons, such as the Sleeping Beauty (SB) transposon system. Thus, in an embodiment, transposons may direct the precise transfer of specific constructs from a donor plasmid into a mammalian chromosome (e.g., reviewed in Hackett, P. B., et al., Adv. in Genet., 54, 189-232 (2005); see also, U.S. Pat. No. 6,489,458)). Using a transposon-based method, the DNA transferred may include the coding sequence for the transposase or, alternatively, the transposase mRNA, in addition to the DNA encoding the a nucleic acid or a polypeptide involved in maintaining a cell having a cosmetic function. For example, the SB transposon is comprised of two terminal repeats of approximately 340 base pairs each, and can mediate transfer of an exogenous nucleotide sequence nearly randomly into chromosomes at TA-dinucleotide base pairs (although flanking DNA sequences may influence the probability of integration at a given site). SB transposons have been used to ameliorate murine disorders that model human disease, and to facilitate somatic integration of an activated NRAS oncogene into mouse hepatocyte DNA (Carelson et al., Proc. Nat. Acad. Sci. U.S.A., 102, 17059-64 (2005)).

In another embodiment, chromosomal integration can be achieved via phage integrases (reviewed in Groth, A. C. & Calos, M. P., J. Biol. Chem., 335(3), 667-678 (2004)). For example, a phage integrase may mediate efficient site-specific recombination between two different, relatively short sequences. Thus, the serine-catalyzed family ΦC31 integrase has been found to work efficiently in human cells to mediate integration at introduced recognition sites or native chromosomal sequences that bear partial identity to these sites (Sclimenti et al., Nucl. Acid Res., 29, 5044-51 (2001)).

### Formulations

The polynucleotide construct encoding at least one of a nucleic acid or a polypeptide involved in maintaining a cell having a cosmetic function so as to enhance and/or maintain a biochemical and/or physiological process that has a positive effect on cosmetic appearance may, in certain embodiments, be mixed with a pharmaceutically acceptable carrier to produce a therapeutic composition that can be administered for the treatment of a cell having a cosmetic function. A variety of formulations that may be used with the methods and/or compositions of the present invention are described in U.S. Patent Publication No. 2006/0058256 and U.S. Patent Publication No. 2006/0025363, both of which are incorporated by reference herein in their entireties.

In an embodiment, the compositions of the present invention may comprise a topical formulation. Topical formulations can be comprised of either dissolving or suspending the compositions in a media such as mineral oil, petroleum, polyhyrodxy alcohols or other bases used for topical pharmaceutical formulations. The addition of other ingredients, such as cocoa butter or aloe may be desirable.

The formulations may include those suitable for oral, rectal, topical, nasal, ophthalmic or parenteral (including subcutaneous, intramuscular and intravenous) administration, all of which may be used as routes of administration for practicing the present invention.

The formulations may conveniently be presented in a dosage form and may be prepared by any of the methods well known in the art of pharmacy. The formulations may include bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Formulations suitable for parenteral administration may conveniently comprise a sterile aqueous preparation of the active compound, which is preferably isotonic with the blood of the recipient.

Nasal spray formulations may comprise purified aqueous solutions of the active compound with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations may be prepared by a similar method to the nasal spray, except that the pH and isotonic factors are preferably adjusted to match that of the eye.

Formulations of the present invention suitable for oral administration may be presented as discrete units such as capsules, cachets, tablets or lozenges, each containing a predetermined amount of a potentiating agent as a powder or granules; as liposomes; or as a suspension in an aqueous liquor or non-aqueous liquid such as a syrup, an elixir, an emulsion or a draught. For example, a tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised of a mixture of the powdered active compound with a suitable carrier may be made by molding in a suitable machine. Or, a syrup may be made by adding the active compound to a concentrated aqueous solution of a sugar, for example sucrose to which may also be added any accessory ingredient(s). Such accessory ingredient(s) may include flavorings, suitable preservatives, an agent to retard crystallization of the sugar, and an agent to increase the solubility of any other ingredient, such as a polyhydric alcohol, for example glycerol or sorbitol.

In an embodiment, administration of the polynucleotide encoding at least one of a nucleic acid or a polypeptide involved in maintaining a cell having a cosmetic function may be topical. Topical formulations may comprise the active compound dissolved or suspended in one or more media such as mineral oil, petroleum, polyhydroxy alcohols or other bases used for topical pharmaceutical formulations. The addition of other accessory ingredients may be desirable. For example, the preparation of the present invention may be in the form of a skin cream, face cream, lotion, ointment, or any other suitable topical skin formulation (see e.g., U.S. Pat. No. 4,760,096 incorporated by reference herein in its entirety). Depending upon the intended use of the preparation, other components can be incorporated into it to prepare a skin preparation having desired rheological properties.

Thus, in an embodiment, the formulations of the present invention may be in the form of an aqueous mixture such as a solution, colloidal solution, emulsified lotion, oil-in-water cream (hydrophilic cream) or aqueous gel wherein the aqueous phase is the continuous phase. Alternatively, the formulation can be in the form of an oily mixture such as a solution, ointment, water-in-oil cream, gel base, absorption base or hydrophilic ointment wherein the oil phase is the continuous phase. Also, a non-aqueous water-soluble base such as a mixture with polyethylene glycol may be used. Also, in an embodiment, a suspension base such as a shaking lotion, in which a solid dispersing agent is added, can also be prepared. Oily components, emulsifiers, dispersing agents, gelatinizers and solid materials which can be used to prepare such formulations are well known for use in the preparation of cosmetics and topical products.

As is known in the art, the oily components may include hydrocarbons such as liquid paraffin, petrolatum, solid paraffin, or microcrystalline wax. Also, higher aliphatic alcohols such as cetyl alcohol, hexadecyl alcohol, stearyl alcohol, oleyl alcohol; esters of higher aliphatic alcohols such as bees wax; esters of higher aliphatic acids with lower alcohols such as isopropyl myristate or isopropyl palmitate; vegetable oils and modified vegetable oils; anhydrous lanolin and its derivatives; squalene, or squalane; and higher aliphatic acids such as palmitic acid, stearic acid may be used.

In an embodiment, the formulations can be used with physical (e.g. dermabrasion and occlusion), and/or chemical permeation/penetration enhancers (Azone, DMSO, alcohols, fatty acids and terpenes), as such penetration enhancers have been shown to increase permeability by disordering or ‘fluidising’ the lipid structure of the stratum corneum). Alternatively or additionally, the formulations of the present invention may be used with other penetration enhancing methods (electroporation, ultrasound, etc.).

In an embodiment L-serine, L-hydroxyproline, or other amino acids that may potentiate and/or synergize with growth factor action may be included in the formulations of the present invention.

In an embodiment, a topical formulation of the present invention may comprise useful emulsifiers and dispersing agents including anionic, cationic and nonionic surfactants. Nonionic surfactants may be preferred because of their low level of irritation to skin. Typical of nonionic surfactants may include monoglycerides such as glyceryl monostearate; sorbitan aliphatic esters such as sorbitan monolaurate; sucrose aliphatic esters; polyoxyethylene aliphatic esters such as polyoxyethylene stearate; and polyoxyethylene higher alcohol ethers such as polyoxyethylene cetyl ether, polyoxyethylene oleyl ether, polyoxyethylene fatty ethers and the like (see e.g., U.S. Pat. No., 4,760,096). Also, gelatinizers such as carboxymethylcellulose, cellulose gel, polyvinyl alcohol, polyethylene glycol and various gums may be used. These oily components, emulsifiers, dispersing agents and gelatinizers can be used alone or in combination with each other. Also, nanoemulsions may be used.

In addition to the aforementioned ingredients, the formulations of this invention may further include one or more accessory ingredient(s) selected from diluents, buffers, flavoring agents, binders, disintegrants, surface active agents, thickeners, lubricants, preservatives (including antioxidants) and the like.

Dosages

The polynucleotide construct may be administered in an amount to deliver the amount of polypeptide or polynucleotide that is required to maintain and/or improve cells having cosmetic function. For topical application of the polypeptide, in alternate embodiments, a dose administration of from about 1 ng/cm² to 1 mg/cm² of tissue area, or from about 10 ng/cm² to 100 μg/cm² of tissue area, or from about 100 ng/cm² to 10 μg/cm² of tissue area, or from about 500 ng/cm² to 10 μg/cm² of tissue area, or from about 1 μg/cm² to 5 μg/cm² of tissue area, or from about 1 μg/cm² to 2 μg/cm² of tissue area may be used.

Or, for systemic administration (e.g., intraperitoneal) the dose may range from about 1 ng/kg/day to 100 mg/kg/day, or from about 10 ng/kg/day to 10 mg/kg/day, or from about 100 ng/kb/day to 5 mg/kg/day, or from about 1 μg/kg/day to 1 mg/kg/day, or from about 100 μg/kg/day to 500 μg/kg/day, or from about 10 μg/kg/day to 100 μg/kg/day. Or, ranges within these ranges may be used.

In alternate embodiments, the polynucleotide may provide the equivalent to a dose administration of polypeptide that ranges from about 1 ng/cm² to 1 mg/cm² of tissue area, or from about 10 ng/cm² to 100 μg/cm² of tissue area, or from about 100 ng/cm² to 10 μg/cm² of tissue area, or from about 500 ng/cm² to 10 μg/cm² of tissue area, or from about 1 μg/cm² to 5 μg/cm² of tissue area, or from about 10 μg/cm² to 2 μg/cm² of tissue area. Or, ranges within these ranges may be used.

For example, vector constructs may be applied as naked DNA doses that range from about 1 ng/cm² to 1 mg/cm² of tissue area, or from about 10 ng/cm² to 10 mg/cm² of tissue area, or from about 100 ng/cm² to 100 μg/cm² of tissue area, or from about 500 ng/cm² to 10 μg/cm² of tissue area, or from about 1 μg/cm² to 5 μg/cm² of tissue area, or from about 10 μg/cm² to 2 μg/cm² of tissue area. Or, ranges within these ranges may be used.

These dose ranges may be determined by performing in vitro and animal studies to precisely characterize the efficiency of gene transfection in the cosmetic cell of interest. Similarly, dose ranges for other carriers, such as liposomes, nanosomes, nanoemulsions, particle-mediated transfer, and voltage driven transfer may be determined using methods specific to each of these applications as is known in the art.

Thus, embodiments of the present invention allow for genetic modification of cells having cosmetic function to enhance and/or maintain expression of genes that may stimulate production of skin proteins for a more youthful appearance. In certain embodiments, the methods and compositions of the present invention may have distinct advantages over currently available cosmetic enhancers including plastic surgery, BOTOX® (injectable botulinum toxin), laser resurfacing, microdermabrasion, pulsed light therapy, injectable fillers/connective tissue substitutes, autologous fibroblast injection, skin care topical products and other methods.

In one advantage, the methods and compositions of the present invention provide improved constant delivery of therapeutic molecules to cells having cosmetic function as compared to topical application of compositions to the such tissues.

Also, in some cases, topically applied compounds, including growth factors, may be rapidly digested by proteases thus limiting the duration of their beneficial effects, or stimulate an immunogenic response. Embodiments of the present invention may stimulate production of the cells having cosmetic function to increase production of the cell's own proteins, and thus, there may be a reduced activation of proteases or an immunogenic response.

Unlike topically applied growth factor protein products which recommend twice daily topical application to achieve improved cosmetic appearance, embodiments of the methods and compositions of the present invention may offer once weekly or once monthly dosing (i.e., the standard daily or twice daily application may not be required for full cosmetic benefits), or in the case of stem cell transfection, one time dosing, to improve cosmetic appearance. Constant round-the-clock steady expression of a cosmetic enhancing polypeptide, for example a plasmid encoding keratinocyte growth factor (KGF), for a seven day period, would have obvious advantages over the very temporal peak and troughs of twice daily topical KGF polypeptide application. In effect, this method turns the skin into a bioreactor producing constant cosmetically beneficial polypeptides with distinct advantages. Also, because the methods and compositions of the present invention are administered much less frequently than standard topical treatments, the methods and compositions of the present invention can eliminate or reduces the need for invasive, and sometimes complicated, surgical, injectable and laser/light therapies which produce skin damage as an inherit part of the treatment, and with improved compliance with less frequent dosing.

### EXAMPLES

The examples described herein describe the synthesis of four recombinant vector constructs that encode proteins designed to improve the stability of the extracellular matrix and support the improved appearance of the skin. These are collagen, elastin, EC-SOD, and TIMP-1. The role of these proteins in skin physiology is discussed in detail herein. Each plasmid construct consists of one gene of interest and plasmid cDNA with a human cytomegalovirus immediate-early promoter designed to provide a high level of stability and transient expression of the gene of interest. The genes incorporated into the plasmid cDNA vectors include collagen 1, elastin, tissue inhibitor of metalloproteinase-1 (TIMP-1), and extracellular superoxide dismutase (EC-SOD). The cDNA plasmid constructs containing the genes of interest will be formulated into a skin cream for topical application.

### Example 1

### Amplification, Cloning, and Expression of Human Collagen α1 Type 1, Collagen α2 Type 1, TIMP-1, and Elastin from Normal Tissue cDNAs

First strand cDNAs generated from normal human tissues were purchased from BioChain Institute (Hayward, Calif.). Collagen α1 Type 1 (COLA1A), Collagen α2 Type 1 (COL1A2), and Elastin were amplified from normal human skin (Cat. No. C1234218-10) cDNAs, whereas TIMP-1 was amplified from both normal human lung (Cat. No. C1234152-10) and brain (Cat. No. C1244035-10) cDNAs. Gene amplifications were performed with high fidelity Platinum Pfx Polymerase (Invitrogen, Carlsbad, Calif.), according to manufacturer's instructions, with gene specific oligonucleotides. Table 2 summarizes oligonucleotide sequences used for amplification of each gene, as well as Polymerase Chain Reaction (PCR) conditions. Table 2 shows the oligonucleotide sequences and PCR parameters used in the amplification of COLA1A, COL1A2, Elastin, and TIMP-1 from normal human tissue cDNAs.

TABLE 2

Primers and PCR paratmeters for cloning COLA1A, COL1A2,
Elastin and TIMP-1 cDNAs from normal tissue

| | | | | |
|---|---|---|---|---|
| Tissue | | | Anneal | Ext. |
| source | 5′ oligo | 3′ oligo | temp. | time |

| | | | | | |
|---|---|---|---|---|---|
| COL | Skin | GATCGCTAGCGCCGC | CGATAAGCTTTTACA | 56° C. | 6 min. |
| A1A | | CACCATGTTCAGCTT | GGAAGCAGACAGGG | | |
| | | TGTGGACCTCCGGCT | CCAACGTCGAAGCC | | |
| | | CCTGC | G | | |
| COL1 | Skin | GATCGCTAGCGCCGC | CGATAAGCTTTTATT | 56° C. | 6 min. |
| A2 | | CACCATGCTCAGCTT | TGAAACAGACTGGG | | |
| | | TGTGGATACGCGGAC | CCAATGTCCACAAAG | | |
| | | TTTGTTGCTGCTT | AATTCCT | | |
| Elastin | Skin | GATCGCTAGCGCCGC | GCTAAGATCTTCATT | 56° C. | 3 min. |
| | | CACCATGGCGGGTCT | TTCTCTTCCGGCCAC | | |
| | | GACGGCGGCGGCCC | AAGCTTTCCCCAGG | | |
| | | CGCGG | | | |
| TIMP-1 | Brain; | GATCGCTAGCGCCGC | CGATAAGCTTTCAGG | 56° C. | 1.5 min. |
| | lung | CACCATGGCCCCCTT | CTATCTGGGACCGCA | | |
| | | TGAGCCCCTGGCTTC | GGGACTGCCAGGTG | | |
| | | TGGCATCCTG | CA | | |

5′ oligonucleotides were designed with an NheI restriction endonuclease (REN) site for cloning into the mammalian expression vector pcDNA3.1+zeo:intA immediately downstream of the PCMV promoter. Kozak sequences for optimal translation initiation were engineered immediately following each NheI site (GCCGCCACCATG) (i.e., nucleotides 11-22 of SEQ ID NO:3). 3′ oligonucleotides were designed with HindIII Restriction Endonuclease Sites (REN sites) for COLA1A, COL1A2, and TIMP-1, and with BglII REN for Elastin for cloning into the same vector immediately preceding the bovine growth hormone polyadenylation (BGHpA) signal sequence. REN sites are underlined, and translation initiation codons (ATG) are double underlined. Stop codons native to each gene immediately precede the REN site in the 3′ oligonucleotide sequences, and are bolded.

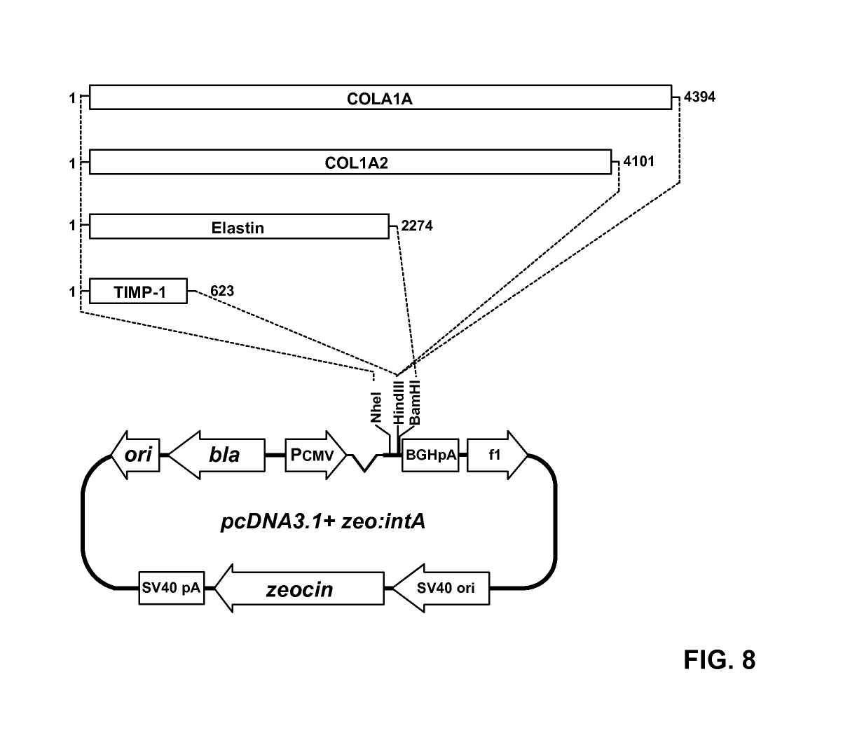

PCR reactions were setup on ice, and contained final concentration of each component, as follows: 1×Pfx Amplification Buffer, 0.3 mM each of dATP, dTTP, dCTP, dGTP, 1 mM MgSO₄, 0.3 μM each oligonucleotide, 1 μL of first strand cDNA template, 1.0-2.5 units Platinum Pfx DNA Polymerase, and nuclease-free distilled water to a final volume of 50 μL. Each reaction was subjected to three-step PCR cycling, using the following parameters: 94° C. for 15 seconds, 56° C. for 30 seconds, and 68° C. for the amount of time indicated in Table 2 above for each gene. Each amplification was performed for 35 cycles, following by a final extension cycle at 72° C. for 7 minutes, and cooling at +4° C. until analysis. A positive control was run alongside each experimental PCR reaction by using the same first strand cDNA template and a set of primers specific for human beta actin (BioChain). Following PCR each reaction was analyzed by Tris-Acetate EDTA (TAE) agarose gel electrophoresis to determine extend and integrity of amplified cDNA. For each analysis 10 μL of each reaction were mixed with Blue Juice loading buffer (Invitrogen) and loaded per lane of a 1% TAE agarose gel. TriDye 1 kb DNA ladder (New England Biolabs, Ipswich, Mass.) was used to estimate the size of amplified cDNAs. Following confirmation of PCR amplification for each cDNA each reaction was immediately cloned into the Zero Blunt TOPO PCR Cloning system (Invitrogen), according to manufacturer's instructions. Colonies were screened either by PCR or REN analysis of purified DNAs from overnight bacterial cultures, or both. pCR-Blunt II-TOPO clones containing fragments of expected sizes were subsequently restricted with the appropriate RENs for isolation and cloning of the full length genes into the mammalian expression vector pcDNA3.1+zeo:intA. The expression vector was constructed from pcDNA3.1+zeo (Invitrogen) by adding the human CMV intron A sequence to the 3′ end of the minimal promoter. CMV intron A sequence has been extensively characterized and shown to greatly enhance the expression of recombinant genes in mammalian cells when compared with an intronless CMV promoter counterpart. The intron A sequence was PCR amplified from the vector pWRG7077 (kindly provided by Dr. Jay Hooper, United States Army Medical Research Institute of Infectious Diseases [USAMRIID]), and was cloned into pcDNA3.1+zeo as a NdeI-NheI sites. pcDNA3.1+zeo:intA is a 6.5 kb plasmid with the expression elements outlined in FIG. 8. TIMP-1 was amplified from both brain and lung tissue cDNAs. For this work TIMP-1 from brain cDNAs was used for all subsequent cloning and expression experiments.

Cloning reactions were performed as described in Maniatis and Sambrook, 1988, followed by transformation into subcloning efficiency Escherichia coli (E. coli) strain DH5α, for propagation of cloned DNAs. Colonies were screened either by PCR or REN analysis of purified DNAs from overnight bacterial cultures, or both. pcDNA3.1+zeo:intA clones containing fragments of expected sizes were subsequently restricted with unique RENs for confirmation of gene identities. For each construct highly pure midiprep plasmid DNAs were isolated form 50 mL E. coli cultures growth overnight in selective broth. DNAs were assayed for purity and concentration by A280 and A260. Expression of each gene was confirmed by lipofectamine-mediated transfection of the human endothelial kidney cell line HEK-293T/17, and subsequent analysis of cell extracts and supernatants. For each construct to be analyzed, 1×10⁶ HEK-293T/17 cells were seeded per well of a Poly-D-Lysine coated 6 well plate in 2 mL of growth medium (DMEM, high glucose; 2 mM L-Glutamine; 1× Non Essential Amino Acids [NEAA]; 10% heat inactivated Fetal Bovine Serum [FBS]) the night prior to transfection, and cultured at 37° C., 5% CO₂, 90% relative humidity (Rh). The following day cultured were fed with 2 mL of fresh growth medium prior to transfection. Four μg of each plasmid DNA construct were gently mixed in 250 μL of Opti-MEM Reduced Serum Medium (Invitrogen) and combined with an additional 250 μL of the same medium supplemented with 10 μL of Lipofectamine 2000 transfection reagent (Invitrogen). The reaction was incubated at room temperature for 20 minutes to allow DNA:lipofectamine complexes to form. The entire reaction was then gently pipetted into the corresponding well containing HEK-293T/17 cells and was gently swirled to evenly distribute the transfection mix. Plates were then returned to the incubator and were cultured for 72 hours prior to harvesting and analysis. Expression of each gene construct was performed on transfected HEK-293T/17 cell extracts and culture supernatants.

Following the 72 hour transfection supernatants were harvested and cleared by centrifugation. One mL of each supernatant was transferred to a polypropylene microcentrifuge tube and kept on ice until analysis. Cells were scraped from wells and were pelleted by brief centrifugation in polypropylene microcentrifuge tubes. Supernatants were carefully decanted and pellets were resuspended in 1× Phosphate Buffered Saline (PBS), pH 7.4, and pelleted by brief centrifugation. PBS was decanted and each cell pellet was resuspended in 100 μL of Mammalian Cell Lysis Buffer (Sigma-Aldrich, St. Louis, Mo.), according to manufacturer's instructions. Lysis buffer was prepared with TRIS buffer, Sodium Chloride, Sodium Dodecyl Sulfate (SDS), Igepal, Deoxycholate, and protease inhibitors. Lysis reactions were incubated at room temperature for 10 minutes, followed by centrifugation at 14,000×g for 10 minutes at room temperature. Supernatants were transferred to fresh polypropylene microcentrifuge tubes and were kept on ice until analysis. SDS-Polyacrylamide Gel Electrophoresis (SDS-PAGE) and Western Blot analysis were performed on each set of transfected HEK-293T/17 cell extracts and supernatants. One hundred thousand cell equivalents (˜10 μL) were mixed with NuPage LDS Sample Buffer, NuPAGE Reducing Agent, and deionized water, heated at 90° C. for 5 minutes and were loaded per lane of a 10% NuPage Novex Bis-Tris Gel. Likewise, 30 μL of corresponding supernatant were similarly prepared and loaded alongside cell extracts. SeeBlue Plus-2 Protein molecular weight marker (Invitrogen) was used to estimate protein sizes. Gels were run in 1×SDS NuPAGE MES Running Buffer at 200 V, for 45 minutes. Following electrophoresis proteins were transferred to 0.45 μm Nitrocellulose membrane using an X-Cell II Blot Module, according to manufacturer's instructions (Invitrogen). Western blots were performed with a Protein Detector TMB Western Blot Kit (KPL, Gaithersburg, Md.), according to manufacturer's instructions, using primary antisera and secondary detection reagents, as outlined in Table 3. Rabbit antisera to COLA1A, COL1A2, TIMP-1, and Elastin were purchased from Santa Cruz Biotechnology (Santa Cruz, Calif.). Horseradish Peroxidase (HRP)-labeled Goat anti-Rabbit IgG (H+L) (Santa Cruz Biotechnology) was used as secondary detection reagent. Immunological complexes were detected with TMB membrane substrate for 2-5 minutes, and reactions were stopped by immersing the blots in distilled water. Permanent records were generated by high resolution scanning of developed blots. Table 3 shows detection reagents for Western Blot analysis of COLA1A, COL1A2, TIMP-1, and Elastin transiently expressed in HEK-293T/17 cells.

TABLE 3
Sera Used for Western Blot Detection of Expressed
Recombinant Proteins

| | | | Secondary |
|---|---|---|---|
| | Primary antisera | Source | detection reagent |
| COLA1A | Rabbit polyclonal | Collagen Type | Goat anti-Rabbit |
| | antibody to amino | 1 (H-197): | IgG (H + L)-HRP: |
| | acids 1021-1217 of | sc-28657 | sc-2004 |
| | Human Collagen α1 | | |
| | Type 1 | | |
| COL1A2 | Rabbit polyclonal | Collagen Type | |
| | antibody to amino | 1 (H-70): | |
| | acids 1021-1090 of | sc-28655 | |
| | Human Collagen α2 | | |
| | Type 1 | | |
| Elastin | Rabbit polyclonal | Elastin (H-300): | |
| | antibody to amino | sc-25736 | |
| | acids 431-730 of | | |
| | Human Elastin | | |
| TIMP-1 | Rabbit polyclonal | TIMP-1 (H- | |
| | antibody to amino | 150): sc-5538 | |
| | acids 58-207 of | | |
| | Human TIMP-1 | | |

FIG. 8 shows the cloning strategy for expression of human proteins COLA1A, COL1A2, Elastin, and TIMP-1 in mammalian cells using the CMV promoter-driven eukaryotic vector pcDNA3.1+zeo:intA. Genes were PCR amplified from normal human tissue cDNAs and cloned as described in Materials and Methods. COLA1A, COL1A2, and TIMP-1 were directionally cloned into the mammalian vector's NheI-Hind III REN sites, whereas Elastin was cloned into NheI-BamHI sites. The BglII site on the 3′ end of Elastin was directionally cloned into the unique isoschizomer site BamHI. Relevant expression elements are bacterial origin of replication (ori), beta-lactamase gene (bla), cytomegalovirus early promoter (P_{CMV}), bovine growth hormone polyadenylation signal (BGHpA), single-stranded philamentous phage origin (f1), simian virus 40 origin of replication (SV40 ori), simian virus 40 polyadenylation signal (SV40 pA), zeocin antibiotic resistance gene (zeocin).

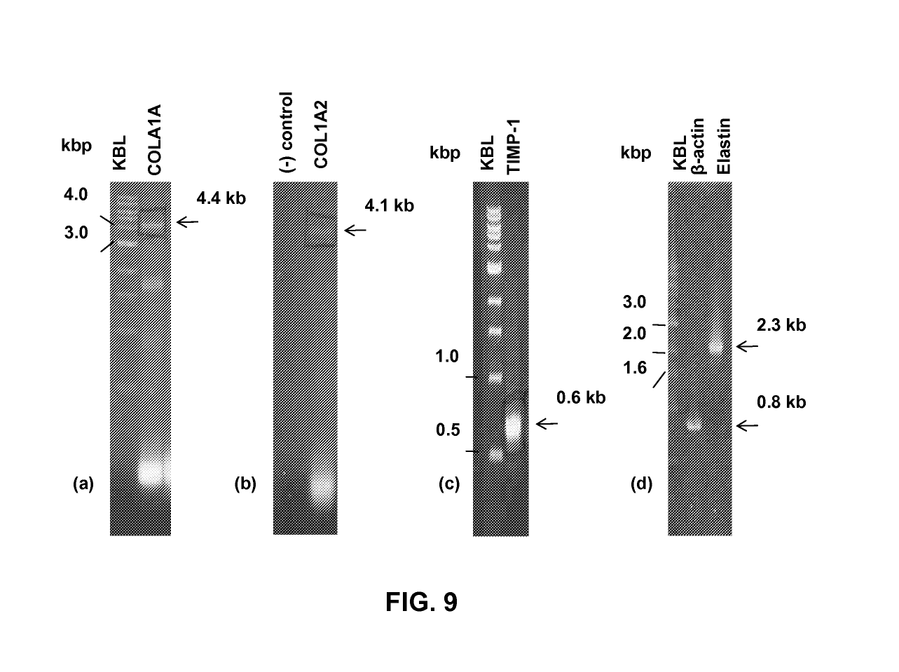

FIG. 9 shows PCR amplification of COLA1A, COL1A2, TIMP-1, and Elastin from normal human tissue cDNAs. Each gene was amplified as described in Materials and Methods using gene-specific oligonucleotide primers. Amplified gene sequences of interested are marked by arrows, and corresponding sizes are indicated. (a) COLA1A; (b) COL1A2; (c) TIMP-1; (d) Elastin. KBL, kilobase ladder; kbp, kilobase pairs.

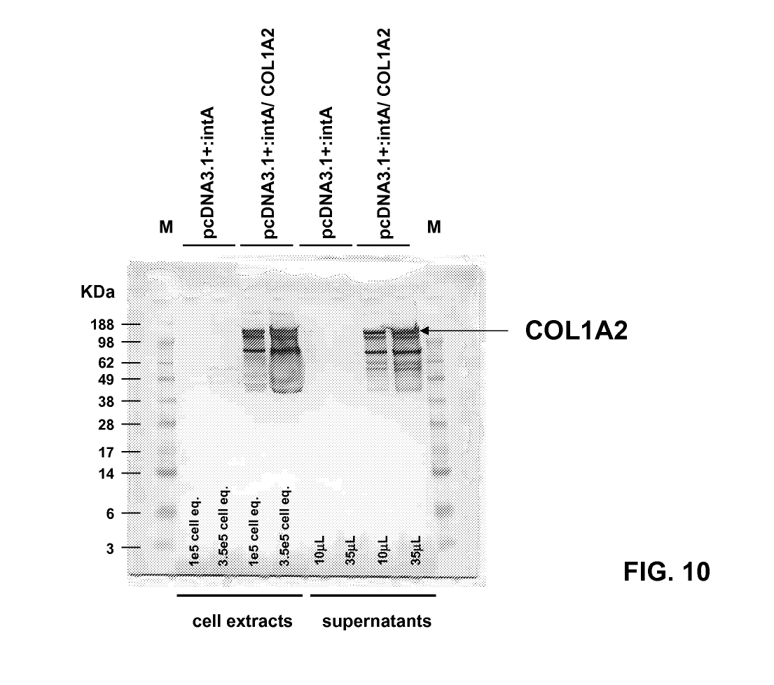

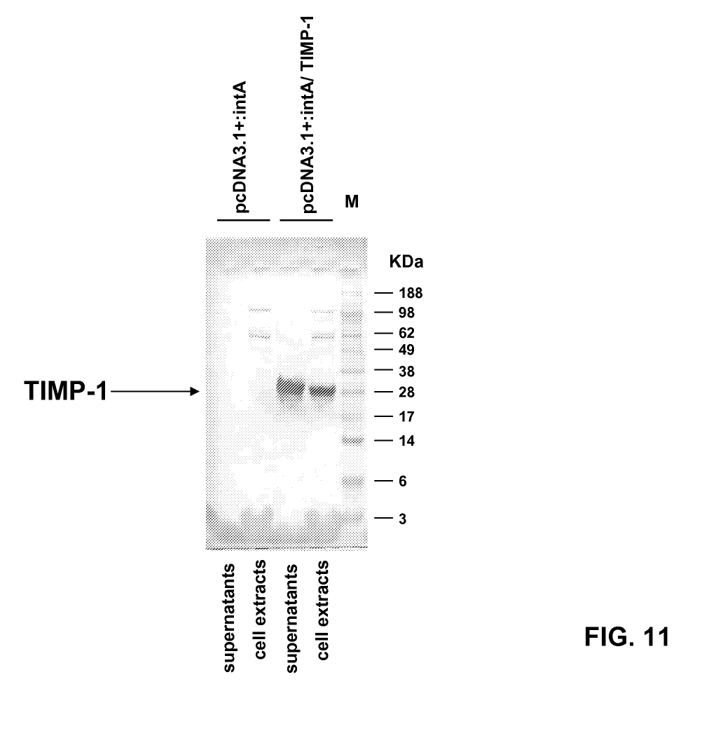

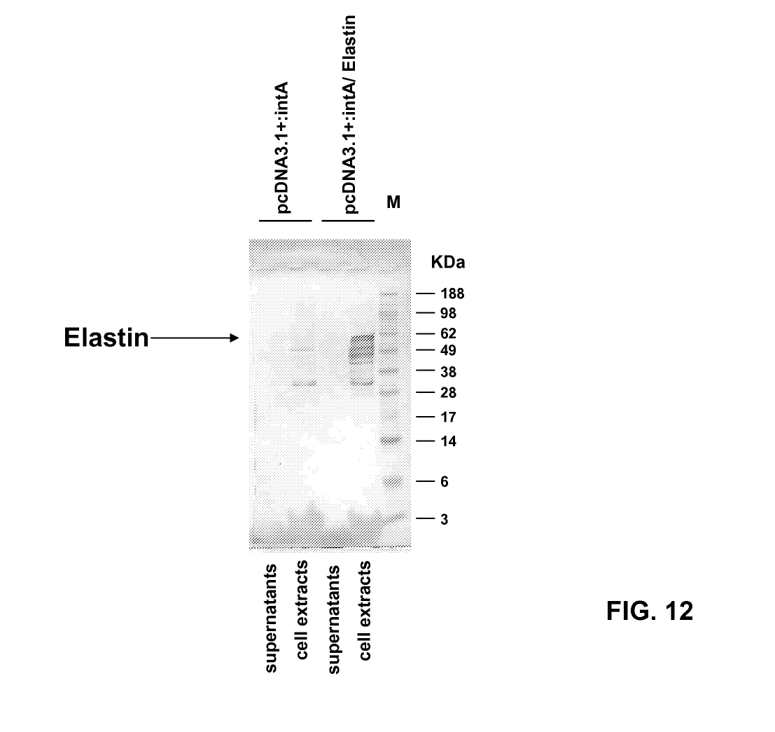

Results showing expression of the cloned proteins (COL1A2, TIMP-1, and Elastin) are shown in FIGS. 10-12. FIG. 10 shows transient expression analysis of human COL1A2 in HEK-293T/17 cells. The 138.9 KDa COL1A2 protein is indicated by an arrow. Minor fragments detected by the COL1A2-specific antiserum may be incomplete translation products and degraded protein. FIG. 11 shows transient expression analysis of human TIMP-1 in HEK-293T/17 cells. The 23.2 KDa TIMP-1 protein is indicated by an arrow. TIMP-1 runs higher than its predicted protein molecular weight of 23 KDa likely due to predicted N-linked and Asn-Xaa-Ser/Thr glycosylation of this protein in mammalian cells. FIG. 12 shows transient expression analysis of human Elastin in HEK-293T/17 cells. The 66.1 KDa Elastin protein is indicated by an arrow. Minor fragments detected by the Elastin-specific antiserum may be incomplete translation products and degraded protein. Expression results indicate that in HEK-293T/17 cells this Elastin construct is not secreted, and is only expressed intracellularly.

All patents, publications and abstracts cited above are incorporated herein by reference in their entirety. It should be understood that the foregoing relates only to certain embodiments of the present invention and that numerous modifications or alterations may be made therein without departing from the spirit and the scope of the present invention as defined in the following claims.

That which is claimed is:

1. A method for the cosmetic genetic modification of substantially intact cells having a cosmetic function in a subject comprising: administering an isolated polynucleotide encoding at least one of a nucleic acid or a polypeptide involved in maintaining the cells having cosmetic function to a least a portion of the cells such that the nucleic acid or polypeptide is expressed in the cells having cosmetic function to enhance and/or maintain a biochemical and/or physiological process that has a positive effect on cosmetic appearance.

2. The method of claim 1, wherein the polynucleotide comprises SEQ ID NO: 1, SEQ ID NO: 5, SEQ ID NO: 9, SEQ ID NO: 13, or SEQ ID NO: 17, or the complement of SEQ ID NO: 1, SEQ ID NO: 5, SEQ ID NO: 9, SEQ ID NO: 13, or SEQ ID NO: 17, or a sequence that is at least 90% identical to SEQ ID NO: 1, SEQ ID NO: 5, SEQ ID NO: 9, SEQ ID NO: 13, or SEQ ID NO: 17, or the complement of SEQ ID NO: 1, SEQ ID NO: 5, SEQ ID NO: 9, SEQ ID NO: 13, or SEQ ID NO: 17.

3. The method of claim 1, wherein the polynucleotide comprises a plasmid or a viral vector.

4. The method of claim 3, wherein the polynucleotide is maintained as a extrachromosomal plasmid in at least a portion of the transfected cells.

5. The method of claim 1, wherein the polypeptide comprises at least one of a keratinocyte growth factor, a transforming growth factor, a platelet derived growth factor, a vascular endothelial growth factor, an insulin-like growth factor, a heptocyte growth factor, a vascular endothelial growth factor, a fibroblast growth factor, an epidermal growth factor, a platelet derived endothelial cell growth factor, a connective tissue growth factor, a granulocyte-macrophage colony-stimulating factor, a macrophage colony stimulating factor, a growth hormone, TSP-1, TSP-2, a collagen protein, TIMP-1, a superoxide dismutase, an elastin, or a hypoxia inducible factor, or a biologically active derivative thereof.

6. The method of claim 1, wherein at least two different polypeptides or nucleic acid molecules are administered to the cells.

7. The method of claim 1, wherein the polynucleotide encoding a nucleic acid or polypeptide involved in maintaining the cells having a cosmetic function is operably linked to a constitutive or an inducible promoter.

8. The method of claim 7, wherein the promoter is not ubiquitously expressed, but is expressed in the cells having cosmetic function.

9. The method of claim 1, wherein the polynucleotide encoding a nucleic acid or polypeptide involved in maintaining the cells having a cosmetic function is operably linked to an enhancer.

10. The method of claim 1, wherein the polynucleotide encoding a nucleic acid or polypeptide involved in maintaining the cells having a cosmetic function is operably linked to at least one of a functional poly A sequence, an intron, a cleavage sequence, a stop sequence, or a cap site.

11. The method of claim 1, wherein the modified cells having a cosmetic function comprise at least one of keratinocytes, fibroblasts, adipocytes, or myofibrils.

12. The method of claim 1, wherein the polynucleotide is introduced as naked DNA into the cells having a cosmetic function.

13. The method of claim 1, wherein the polynucleotide is introduced into the cells having a cosmetic function via at least one of liposomes, nanoparticles, an emulsion, a thixogel, or an organoleptic gel.

14. The method of claim 1, wherein the polynucleotide is introduced into the cells having a cosmetic function via a water-in-oil emulsion or an oil-in-water emulsion.

15. The method of claim 1, wherein the polynucleotide is introduced into the cells having a cosmetic function via at least one of particle mediated transfer (e.g., gold particles, microspheres), voltage driven transfer, radio frequency ablation-mediated transfer, or ultrasound, microneedles.

16. A composition for genetically modifying substantially intact cells having cosmetic function in a subject comprising:
a polynucleotide encoding at least one of an isolated nucleic acid or a polypeptide involved in maintaining cells having cosmetic function; and
a carrier for administration of the polynucleotide to a least a portion of the subject's cells having cosmetic function such that the nucleic acid or polypeptide is expressed in the cells having cosmetic function so as to enhance and/or maintain a biochemical and/or physiological process that has a positive effect on cosmetic appearance.

17. The composition of claim 6, wherein the polynucleotide comprises SEQ ID NO: 1, SEQ ID NO: 5, SEQ ID NO: 9, SEQ ID NO: 13, or SEQ ID NO: 17, or the complement of SEQ ID NO: 1, SEQ ID NO: 5, SEQ ID NO: 9, SEQ ID NO: 13, or SEQ ID NO: 17, or a sequence that is at least 90% identical to SEQ ID NO: 1, SEQ ID NO: 5, SEQ ID NO: 9, SEQ ID NO: 13, or SEQ ID NO: 17, or the complement of SEQ ID NO: 1, SEQ ID NO: 5, SEQ ID NO: 9, SEQ ID NO: 13, or SEQ ID NO: 17.

18. The composition of claim 16, wherein the polynucleotide comprises a plasmid or a viral vector.

19. The composition of claim 18, wherein the polynucleotide is maintained as a extrachromosomal plasmid in at least a portion of the transfected cells.

20. The composition of claim 16, wherein the polypeptide comprises at least one of a keratinocyte growth factor, a transforming growth factor, a platelet derived growth factor, a vascular endothelial growth factor, an insulin-like growth factor, a heptocyte growth factor, a vascular endothelial growth factor, a fibroblast growth factor, an epidermal growth factor, a platelet derived endothelial cell growth factor, a connective tissue growth factor, a granulocyte-macrophage colony-stimulating factor, a macrophage colony stimulating factor, a growth hormone, TSP-1, TSP-2, a collagen protein, TIMP-1, a superoxide dismutase, an elastin, or a hypoxia inducible factor, or a biologically active derivative thereof.

21. The composition of claim 16, comprising at least two different polypeptides or nucleic acid molecules.

22. The composition of claim 16, wherein the polynucleotide encoding a nucleic acid or polypeptide involved in maintaining cells having a cosmetic function is operably linked to a constitutive or an inducible promoter.

23. The composition of claim 16, wherein the polynucleotide encoding a nucleic acid or polypeptide involved in maintaining cells having a cosmetic function is operably linked to an enhancer.

24. The composition of claim 16, wherein the polynucleotide encoding a nucleic acid or polypeptide involved in maintaining cells having a cosmetic function is operably linked to at least one of a functional poly A sequence, an intron, a cleavage sequence, a stop sequence, or a cap site.

25. The composition of claim 16, wherein the polynucleotide is naked DNA mixed with the carrier.

26. The composition of claim 16, wherein the carrier comprises at least one of liposomes, nanoparticles, an emulsion, a thixogel, or an organoleptic gel.

27. The composition of claim 16, wherein the carrier comprises at least one of a water-in-oil emulsion or an oil-in-water emulsion.

28. The method of claim 1, wherein the carrier comprises a formulation suitable for at least one of particle mediated transfer, voltage driven transfer, radio frequency ablation-mediated transfer, or ultrasound, or microneedles.

29. The composition of claim 16, wherein the nucleic acid comprises a regulatory element, an anti-sense RNA, or an inhibitory RNA.

30. An isolated polynucleotide encoding at least one of a nucleic acid or a polypeptide involved in maintaining the cells having cosmetic function.

31. The isolated polynucleotide of claim 30, wherein the polynucleotide comprises SEQ ID NO: 1, SEQ ID NO: 5, SEQ ID NO: 9, SEQ ID NO: 13, or SEQ ID NO: 17, or the complement of SEQ ID NO: 1, SEQ ID NO: 5, SEQ ID NO: 9, SEQ ID NO: 13, or SEQ ID NO: 17, or a sequence that is at least 90% identical to SEQ ID NO: 1, SEQ ID NO: 5, SEQ ID NO: 9, SEQ ID NO: 13, or SEQ ID NO: 17, or the complement of SEQ ID NO: 1, SEQ ID NO: 5, SEQ ID NO: 9, SEQ ID NO: 13, or SEQ ID NO: 17.
